SEQUENCE LISTING (53 sequences)

SEQ ID NO: 1 - DNA, 981 bp, synthetic construct
  misc_feature 1..981: note = Codon-optimised for Saccharomyces cerevisiae
  source 1..981: mol_type = other DNA
  CDS 1..981: note = delta-11-desaturase
atggttccaaacaagggttcctctgatgttttgtctgaacattctgaaccacaattcaccaagttgattgctccacaagctggtccaagaaagtacaaaatcgtttacagaaacttgttgaccttcggttactggcatttgtctgctgtttatggtttgtacttgtgtttcacttgtgctaagtgggctactattttgttcgctttcttcttgtacgttatcgccgaaattggtattactggtggtgctcatagattatgggctcatagaacttacaaagccaagttgccattggaaatcttgttgttgatcatgaactccattgccttccaagatactgcttttacttgggctagagatcatagattgcatcacaagtactctgatactgatgctgatccacataatgctactagaggtttcttctactctcatgttggttggttgttggttaagaaacacccagaagttaaggctagaggtaagtacttgtctttggatgacttgaagaacaaccctttgttgaagttccaaaagaagtacgccattttggtcattggtactttgtgctttttgatgccaactttcgttccagtttacttttggggtgaaggtatttctactgcctggaacattaacttgttaagatacgtcatgaacttgaacatgacctttttggttaactccgctgctcatatttttggtaacaagccatacgataagtctatcgcctctgttcaaaacatctctgtttctttggctactttcggtgaaggtttccataactaccatcatacttatccatgggattacagagctgctgaattgggtaacaatagattgaatatgaccaccgccttcattgatttctttgcttggattggttgggcctacgatttgaaatctgttccacaagaagctattgctaagagatgtgctaaaactggtgatggtactgatatgtggggtagaaagagatga

SEQ ID NO: 2 - protein, 326 aa, synthetic construct
  REGION 1..326: note = Synthetic Construct
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 3 - DNA, 1377 bp, synthetic construct
  misc_feature 1..1377: note = Codon-optimised for Saccharomyces cerevisiae
  source 1..1377: mol_type = other DNA
  CDS 1..1377: note = Ase_FAR
atgccagtcttgacttctagagaagacgaaaaattgtccgtcccagaattttacgctggtaagtctatttttgttaccggtggtactggtttcttgggtaaggtttttatcgaaaagttgttgtactgctgcccagatatcgataagatctacatgttgatcagagaaaaaaagaacttgtccatcgacgaaagaatgtccaagtttttggatgaccctttgttctccagattgaaagaagaaagaccaggtgacttggaaaagatcgttttgattccaggtgatattaccgctcctaatttgggtttgtctgctgaaaacgaaagaatcttgttggaaaaggtcagtgtcattattaactctgctgctaccgttaagttcaacgaaccattgccaattgcttggaagattaacgttgaaggtactagaatgttgttggccttgtctagaagaatgaagagaatcgaagttttcatccatatctccaccgcttactctaatgcttcttctgatagaattgtcgttgacgaaatcttgtatccagctccagctgatatggatcaagtttatcaattggttaaggacggtgtcactgaagaagaaaccgaaagattattgaacggtttgccaaacacttacactttcactaaggctttgaccgaacatttggttgctgaacatcaaacttacgttccaaccattatcatcagaccatctgttgttgcctccattaaggatgaacctattagaggttggttgtgtaattggtttggtgctactggtatttctgttttcactgctaagggtttgaacagagttttgttgggtaaagcctctaacatcgttgatgttatcccagttgattacgttgccaacttggttatagttgctggtgctaaatctggtggtcaaaagtctgatgaattgaaaatctacaactgctgctcctctgactgtaatccagttactttgaagaagatcatcaaagaattcaccgaagataccatcaagaacaagtcccatattatgccattgccaggttggttcgtttttactaagtacaaatggttgttgactttgttgaccatcatcttccaaatgttgccaatgtatttggccgatgtttacagagtcttgaccggtaaaattccaagatatatgaagttgcaccacttggtcattcaaaccagattgggtattgatttcttcacctctcattcttgggttatgaagaccgatagagtcagagaattattcggttctttgtccttggccgaaaaacacatgtttccatgtgatccatcttccattgattggaccgattacttgcaatcttactgctatggtgtcagaagattcttagaaaagaagaagtaa

SEQ ID NO: 4 - protein, 458 aa, synthetic construct
  REGION 1..458: note = Synthetic Construct
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 5 - DNA, 1365 bp, synthetic construct
  misc_feature 1..1365: note = Codon-optimised for Saccharomyces cerevisiae
  sig_peptide 1354..1365: note = KKYR signal peptide
  source 1..1365: mol_type = other DNA
  CDS 1..1365: note = Har_FAR_KKYR
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcacagatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacatttcttggaaaaaaagtacagataa

SEQ ID NO: 6 - protein, 454 aa, synthetic construct
  REGION 1..454: note = Synthetic Construct
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKYR

SEQ ID NO: 7 - DNA, 1368 bp, synthetic construct
  misc_feature 1..1368: note = Codon-optimised for Saccharomyces cerevisiae
  source 1..1368: mol_type = other DNA
  CDS 1..1368: note = Har_FAR
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcacagatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacatttcttggaaaaaaaaagctacgaataa

SEQ ID NO: 8 - protein, 455 aa, synthetic construct
  REGION 1..455: note = Synthetic Construct
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKSYE

SEQ ID NO: 9 - DNA, 1365 bp, synthetic construct
  misc_feature 1..1365: note = Codon-optimised for Saccharomyces cerevisiae
  source 1..1365: mol_type = other DNA
  CDS 1..1365: note = Har_FAR_HDEL
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcacagatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacatttcttggaacatgatgaattgtaa

SEQ ID NO: 10 - protein, 454 aa, synthetic construct
  REGION 1..454: note = Synthetic Construct
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEHDEL

SEQ ID NO: 11 - DNA, 1371 bp, synthetic construct
  misc_feature 1..1371: note = Codon-optimised for Saccharomyces cerevisiae
  source 1..1371: mol_type = other DNA
  CDS 1..1371: note = Has_FAR
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 12 - protein, 456 aa, synthetic construct
  REGION 1..456: note = Synthetic Construct
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 13 - DNA, 1365 bp, synthetic construct
  misc_feature 1..1365: note = Codon-optimised for Saccharomyces cerevisiae
  sig_peptide 1354..1365: note = HDEL
  source 1..1365: mol_type = other DNA
  CDS 1..1365: note = Has_FAR_HDEL
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaacatgatgaattgtaa

SEQ ID NO: 14 - protein, 454 aa, synthetic construct
  REGION 1..454: note = Synthetic Construct
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEHDEL

SEQ ID NO: 15 - DNA, 1362 bp, synthetic construct
  misc_feature 1..1362: note = Codon-optimised for Saccharomyces cerevisiae
  source 1..1362: mol_type = other DNA
  CDS 1..1362: note = Hs_FAR
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 16 - protein, 453 aa, synthetic construct
  REGION 1..453: note = Synthetic Construct
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 17 - DNA, 1362 bp, synthetic construct
  misc_feature 1..1362: note = Codon-optimised for Saccharomyces cerevisiae
  sig_peptide 1351..1362: note = HDEL
  source 1..1362: mol_type = other DNA
  CDS 1..1362: note = Hs_FAR_HDEL
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttgcatgatgaattgtaa

SEQ ID NO: 18 - protein, 453 aa, synthetic construct
  REGION 1..453: note = Synthetic Construct
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLHDEL

SEQ ID NO: 19 - DNA, 37 bp, synthetic construct
  misc_feature 1..37: note = PR-8330 (Ase_FAR_U1_fw)
  primer_bind 1..37: note = PR-8330 (Ase_FAR_U1_fw)
  source 1..37: mol_type = other DNA
  modified_base 9: mod_base = OTHER; note = uracil
agtgcaggtaaaacaatgccagtcttgacttctagag

SEQ ID NO: 20 - DNA, 25 bp, synthetic construct
  misc_feature 1..25: note = Primer PR-8331 (Ase_FAR_U1_rev)
  primer_bind 1..25: note = PR-8331 (Ase_FAR_U1_rev)
  source 1..25: mol_type = other DNA
  modified_base 8: mod_base = OTHER; note = uracil
cgtgcgatttacttcttcttttcta

SEQ ID NO: 21 - DNA, 37 bp, synthetic construct
  misc_feature 1..37: note = PR-8332 (Har_FAR_U1_fw)
  primer_bind 1..37: note = PR-8332 (Har_FAR_U1_fw)
  source 1..37: mol_type = other DNA
  modified_base 9: mod_base = OTHER; note = uracil
agtgcaggtaaaacaatggttgtcttgacctccaaag

SEQ ID NO: 22 - DNA, 32 bp, synthetic construct
  misc_feature 1..32: note = PR-8336 (Hs_FAR_U1_fw)
  primer_bind 1..32: note = PR-8336 (Hs_FAR_U1_fw)
  source 1..32: mol_type = other DNA
  modified_base 9: mod_base = OTHER; note = uracil
agtgcaggtaaaacaatggttgtcttgacctc

SEQ ID NO: 23 - DNA, 25 bp, synthetic construct
  misc_feature 1..25: note = PR-8337 (Hs_FAR_U1_rev)
  primer_bind 1..25: note = PR-8337 (Hs_FAR_U1_rev)
  source 1..25: mol_type = other DNA
  modified_base 8: mod_base = OTHER; note = uracil
cgtgcgatttaagtctttttttcca

SEQ ID NO: 24 - DNA, 32 bp, synthetic construct; identical to SEQ ID NO: 22
  misc_feature 1..32: note = PR-8340 (Has_FAR_U1_fw)
  primer_bind 1..32: note = PR-8340 (Has_FAR_U1_fw)
  source 1..32: mol_type = other DNA
  modified_base 9: mod_base = OTHER; note = uracil

SEQ ID NO: 25 - DNA, 25 bp, synthetic construct
  misc_feature 1..25: note = PR-8341 (Has_FAR_U1_rev)
  primer_bind 1..25: note = PR-8341 (Has_FAR_U1_rev)
  source 1..25: mol_type = other DNA
  modified_base 8: mod_base = OTHER; note = uracil
cgtgcgatttacttgttggtagtct

SEQ ID NO: 26 - DNA, 36 bp, synthetic construct
  misc_feature 1..36: note = PR-8350 (Atrd11_U1_fw)
  primer_bind 1..36: note = PR-8350 (Atrd11_U1_fw)
  source 1..36: mol_type = other DNA
  modified_base 9: mod_base = OTHER; note = uracil
agtgcaggtaaaacaatggttccaaacaagggttcc

SEQ ID NO: 27 - DNA, 25 bp, synthetic construct
  misc_feature 1..25: note = PR-8351 (Atrd11_U1_rev)
  primer_bind 1..25: note = PR-8351 (Atrd11_U1_rev)
  source 1..25: mol_type = other DNA
  modified_base 8: mod_base = OTHER; note = uracil
cgtgcgattcatctctttctacccc

SEQ ID NO: 28 - DNA, 33 bp, synthetic construct
  misc_feature 1..33: note = PR-10350 (atf1_U1_fw)
  primer_bind 1..33: note = PR-10350 (atf1_U1_fw)
  source 1..33: mol_type = other DNA
  modified_base 9: mod_base = OTHER; note = uracil
agtgcaggtaaaacaatgaatgaaatcgatgag

SEQ ID NO: 29 - DNA, 33 bp, synthetic construct
  misc_feature 1..33: note = PR-10351 (atf1_U1_rev)
  primer_bind 1..33: note = PR-10351 (atf1_U1_rev)
  source 1..33: mol_type = other DNA
  modified_base 8: mod_base = OTHER; note = uracil
cgtgcgatctaagggcctaaaaggagagctttg

SEQ ID NO: 30 - DNA, 46 bp, synthetic construct
  misc_feature 1..46: note = PR-10738 (Har_FAR_KKSYE_U1_rev)
  primer_bind 1..46: note = PR-10738 (Har_FAR_KKSYE_U1_rev)
  source 1..46: mol_type = other DNA
  modified_base 8: mod_base = OTHER; note = uracil
cgtgcgatttattcgtagctttttttttccaagaaatgtctaacac

SEQ ID NO: 31 - DNA, 43 bp, synthetic construct
  misc_feature 1..43: note = PR-10739 (Har_FAR_HDEL_U1_rev)
  primer_bind 1..43: note = PR-10739 (Har_FAR_HDEL_U1_rev)
  source 1..43: mol_type = other DNA
  modified_base 8: mod_base = OTHER; note = uracil
cgtgcgatttacaattcatcatgttccaagaaatgtctaacac

SEQ ID NO: 32 - DNA, 43 bp, synthetic construct
  misc_feature 1..43: note = PR-10740 (Hs_FAR_HDEL_U1_rev)
  primer_bind 1..43: note = PR-10740 (Hs_FAR_HDEL_U1_rev)
  source 1..43: mol_type = other DNA
  modified_base 8: mod_base = OTHER; note = uracil
cgtgcgatttacaattcatcatgcaagaaatgtctaacacccc

SEQ ID NO: 33 - DNA, 43 bp, synthetic construct
  misc_feature 1..43: note = PR-10741 (Has_FAR_HDEL_U1_rev)
  primer_bind 1..43: note = PR-10741 (Has_FAR_HDEL_U1_rev)
  source 1..43: mol_type = other DNA
  modified_base 8: mod_base = OTHER; note = uracil
cgtgcgatttacaattcatcatgttccaagaagtgtctaacac

SEQ ID NO: 34 - DNA, 2103 bp, Saccharomyces cerevisiae
  source 1..2103: mol_type = other DNA
  CDS 1..2103: note = FAA1
atggttgctcaatataccgttccagttgggaaagccgccaatgagcatgaaactgctccaagaagaaattatcaatgccgcgagaagccgctcgtcagaccgcctaacacaaagtgttccactgtttatgagtttgttctagagtgctttcagaagaacaaaaattcaaatgctatgggttggagggatgttaaggaaattcatgaagaatccaaatcggttatgaaaaaagttgatggcaaggagacttcagtggaaaagaaatggatgtattatgaactatcgcattatcattataattcatttgaccaattgaccgatatcatgcatgaaattggtcgtgggttggtgaaaataggattaaagcctaatgatgatgacaaattacatctttacgcagccacttctcacaagtggatgaagatgttcttaggagcgcagtctcaaggtattcctgtcgtcactgcctacgatactttgggagagaaagggctaattcattctttggtgcaaacggggtctaaggccatttttaccgataactctttattaccatccttgatcaaaccagtgcaagccgctcaagacgtaaaatacataattcatttcgattccatcagttctgaggacaggaggcaaagtggtaagatctatcaatctgctcatgatgccatcaacagaattaaagaagttagacctgatatcaagacctttagctttgacgacatcttgaagctaggtaaagaatcctgtaacgaaatcgatgttcatccacctggcaaggatgatctttgttgcatcatgtatacgtctggttctacaggtgagccaaagggtgttgtcttgaaacattcaaatgttgtcgcaggtgttggtggtgcaagtttgaatgttttgaagtttgtgggcaataccgaccgtgttatctgttttttgccactagctcatatttttgaattggttttcgaactattgtccttttattggggggcctgcattggttatgccaccgtaaaaactttaactagcagctctgtgagaaattgtcaaggtgatttgcaagaattcaagcccacaatcatggttggtgtcgccgctgtttgggaaacagtgagaaaagggatcttaaaccaaattgataatttgcccttcctcaccaagaaaatcttctggaccgcgtataataccaagttgaacatgcaacgtctccacatccctggtggcggcgccttaggaaacttggttttcaaaaaaatcagaactgccacaggtggccaattaagatatttgttaaacggtggttctccaatcagtcgggatgctcaggaattcatcacaaatttaatctgccctatgcttattggttacggtttaaccgagacatgcgctagtaccaccatcttggatcctgctaattttgaactcggcgtcgctggtgacctaacaggttgtgttaccgtcaaactagttgatgttgaagaattaggttattttgctaaaaacaaccaaggtgaagtttggatcacaggtgccaatgtcacgcctgaatattataagaatgaggaagaaacttctcaagctttaacaagcgatggttggttcaagaccggtgacatcggtgaatgggaagcaaatggccatttgaaaataattgacaggaagaaaaacttggtcaaaacaatgaacggtgaatatatcgcactcgagaaattagagtccgtttacagatctaacgaatatgttgctaacatttgtgtttatgccgaccaatctaagactaagccagttggtattattgtaccaaatcatgctccattaacgaagcttgctaaaaagttgggaattatggaacaaaaagacagttcaattaatatcgaaaattatttggaggatgcaaaattgattaaagctgtttattctgatcttttgaagacaggtaaagaccaaggtttggttggcattgaattactagcaggcatagtgttctttgacggcgaatggactccacaaaacggttttgttacgtccgctcagaaattgaaaagaaaagacattttgaatgctgtcaaagataaagttgacgccgtttatagttcgtcttaa

SEQ ID NO: 35 - protein, 700 aa, Saccharomyces cerevisiae
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 36 - DNA, 2076 bp, Yarrowia lipolytica
  source 1..2076: mol_type = other DNA
  CDS 1..2076: note = FAA
atggtcggatacacaatttcctcaaagcccgtgtcggtggaggtcggccccgccaagcctggcgagactgccccccgacgaaacgtcattgccaaggacgcccctgtcgtcttccccgacaacgactcgtccctgaccaccgtctacaagctgttcaaaaagtacgccgagatcaacagcgagcgaaaggccatgggatggcgagacaccatcgacatccacgtggagaccaaacaggtgaccaaggtcgtggacggagtggagaagaaggtgcccaaggaatggaagtactttgagatgggcccttacaagtggctctcatacaaggaggcccttaagctggtccatgattatggagctggtcttcgacacctcggaatcaagcccaaggagaagatgcacatttacgcccagacctcccaccgatggatgctctctggcctggcttctctgtctcagggtattcccattgtcactgcctacgacactcttggagaggagggtctcactcgatctctccaggagaccaactcggtcatcatgtttaccgacaaggctctgctgagctctctcaaggtctctctcaagaagggcaccgatctgcgaatcatcatctacggaggtgatctgacccccgacgacaagaaggccggaaacacggagattgacgccatcaaggagattgttccagatatgaagatctacaccatggacgaggttgtcgctctcggccgagaacacccccaccccgtggaggaggtcgactatgaggacctggccttcatcatgtacacctctggttctaccggtgtccccaagggtgtggttctgcagcacaagcagatcctcgcctctgtggccggtgtcaccaagatcattgaccgatctatcatcggcaacacagaccggcttctcaacttcctgcccctcgcacacattttcgagtttgtgttcgagatggtcaccttctggtggggtgcttctctgggttacggaaccgtcaagaccatttccgatctgtccatgaagaactgtaagggagacattcgagagctcaagcccaccatcatggtcggcgttcccgctgtctgggaacctatgcgaaagggtattcttggcaagatcaaggagctgtctcctctgatgcagcgggtcttctgggcctcatttgccgccaagcagcgtctcgacgagaacggactccctggtggatctatcctcgactcgctcattttcaagaaggtcaaggacgccactggaggctgtctccgatacgtgtgtaacggaggtgctccagtatctgtcgacacccagaagttcatcaccactctcatctgtcccatgctgattggatgcggtctgaccgagactacagccaacaccaccatcatgtcgcctaaatcgtacgcctttggcaccattggtgagcccaccgccgccgtgaccctcaagctcattgacgtgcctgaagccggctacttcgccgagaacaaccagggagagctgtgcatcaagggcaacgtcgtgatgaaggagtactacaagaacgaggaggagaccaagaaggcgttctccgacgatggctatttcctcaccggtgatattgccgagtggaccgccaatggccagctcagaatcattgaccgacgaaagaacctcgtcaagacccagaacggagagtacattgctctggagaagctcgagacacagtaccgatcgtcgtcgtacgtggccaacctgtgtgtgtacgccgaccagaaccgagtcaagcccattgctctggtcattcctaacgagggccccaccaagaagcttgcccagagcttgggcgtcgattctgacgactgggacgccgtctgttccaacaaaaaggtggtcaaggctgtgctcaaggacatgctcgataccggccgatctctgggtctgtccggcattgagctgctgcaaggcattgtgttgctgcctggcgagtggactcctcagaacagctacctgactgctgcccagaagctcaaccgaaagaagattgtggatgataacaagaaggaaattgatgagtgctacgagcagtcttag

SEQ ID NO: 37 - protein, 691 aa, Yarrowia lipolytica
MVGYTISSKPVSVEVGPAKPGETAPRRNVIAKDAPVVFPDNDSSLTTVYKLFKKYAEINSERKAMGWRDTIDIHVETKQVTKVVDGVEKKVPKEWKYFEMGPYKWLSYKEALKLVHDYGAGLRHLGIKPKEKMHIYAQTSHRWMLSGLASLSQGIPIVTAYDTLGEEGLTRSLQETNSVIMFTDKALLSSLKVSLKKGTDLRIIIYGGDLTPDDKKAGNTEIDAIKEIVPDMKIYTMDEVVALGREHPHPVEEVDYEDLAFIMYTSGSTGVPKGVVLQHKQILASVAGVTKIIDRSIIGNTDRLLNFLPLAHIFEFVFEMVTFWWGASLGYGTVKTISDLSMKNCKGDIRELKPTIMVGVPAVWEPMRKGILGKIKELSPLMQRVFWASFAAKQRLDENGLPGGSILDSLIFKKVKDATGGCLRYVCNGGAPVSVDTQKFITTLICPMLIGCGLTETTANTTIMSPKSYAFGTIGEPTAAVTLKLIDVPEAGYFAENNQGELCIKGNVVMKEYYKNEEETKKAFSDDGYFLTGDIAEWTANGQLRIIDRRKNLVKTQNGEYIALEKLETQYRSSSYVANLCVYADQNRVKPIALVIPNEGPTKKLAQSLGVDSDDWDAVCSNKKVVKAVLKDMLDTGRSLGLSGIELLQGIVLLPGEWTPQNSYLTAAQKLNRKKIVDDNKKEIDECYEQS

SEQ ID NO: 38 - DNA, 1578 bp, Saccharomyces cerevisiae
  source 1..1578: mol_type = other DNA
  CDS 1..1578: note = ATF1
atgaatgaaatcgatgagaaaaatcaggcccccgtgcaacaagaatgcctgaaagagatgattcagaatgggcatgctcggcgtatgggatctgttgaagatctgtatgttgctctcaacagacaaaacttatatcgaaacttctgcacatatggagaattgagtgattactgtactagggatcagctcacattagctttgagggaaatctgcctgaaaaatccaactcttttacatattgttctaccaacaagatggccaaatcatgaaaattattatcgcagttccgaatactattcacggccacatccagtgcatgattatatttcagtattacaagaattgaaactgagtggtgtggttctcaatgaacaacctgagtacagtgcagtaatgaagcaaatattagaagaattcaaaaatagtaagggttcctatactgcaaaaatttttaaacttactaccactttgactattccttactttggaccaacaggaccgagttggcggctaatttgtcttccagaagagcacacagaaaagtggaaaaaatttatctttgtatctaatcattgcatgtctgatggtcggtcttcgatccacttttttcatgatttaagagacgaattaaataatattaaaactccaccaaaaaaattagattacattttcaagtacgaggaggattaccaattattgaggaaacttccagaaccgatcgaaaaggtgatagactttagaccaccgtacttgtttattccgaagtcacttctttcgggtttcatctacaatcatttgagattttcttcaaaaggtgtctgtatgagaatggatgatgtggaaaaaaccgatgatgttgtcaccgagatcatcaatatttcaccaacagaatttcaagcgattaaagcaaatattaaatcaaatatccaaggtaagtgtactatcactccgtttttacatgtttgttggtttgtatctcttcataaatggggtaaatttttcaaaccattgaacttcgaatggcttacggatatttttatccccgcagattgccgctcacaactaccagatgatgatgaaatgagacagatgtacagatatggcgctaacgttggatttattgacttcaccccctggataagcgaatttgacatgaatgataacaaagaaaatttttggccacttattgagcactaccatgaagtaatttcggaagctttaagaaataaaaagcatctccatggcttagggttcaatatacaaggcttcgttcaaaaatatgtgaacattgacaaggtaatgtgcgatcgtgccatcgggaaaagacgcggaggtacattgttaagcaatgtaggtctgtttaatcagttagaggagcccgatgccaaatattctatatgcgatttggcatttggccaatttcaaggatcctggcaccaagcattttccttgggtgtttgttcgactaatgtaaaggggatgaatattgttgttgcttcaacaaagaatgttgttggtagtcaagaatctctcgaagagctttgctccatttacaaagctctccttttaggcccttag

SEQ ID NO: 39 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 40 - DNA, 1470 bp, Spodoptera littoralis
  mRNA 1..1470: note = mRNA coding sequence of delta 11 desaturase
  source 1..1470: mol_type = other DNA
ggacactgacatggactgaaggagtagagaatcggcccgtggagttggccttcattttcagtcttatctctcggtgttatggtagtcacttatatcggtattaaaataagtgaataaggcttgtaaaaatggcgcaatgtgtacaaacaacaacgattttggaacaaaaagaagagaaaacagtaactttgctggtacctcaagcgggaaagaggaagtttgaaattgtgtattttaatatcatcaccttcgcttactggcatatagctggactatatggcctttatttgtgcttcacttcaacaaaatgggcgacagttttattctcattctttctattcgtcgtagcagaagtaggggtcacggctggctcccacagactttggtcgcataaaacttacaaagcaaaactacctttacaaattctgctaatggtgatgaattcccttgcatttcaaaacacagtcattgattgggtgagagaccatcgactccatcataagtatagcgacactgatgccgatccccataatgcctcccgaggatttttctattcgcacgtcggttggctgcttgtgagaaaacaccctgatgtcaagaaacgaggaaaggaaattgatatatctgatatttacaacaatccggtactgaggttccagaagaagtacgcaattcctttcatcggggcagtttgtttcgtcttaccaacattgataccggtttacggttggggagaaacctggactaatgcctggcacgtcgccatgctgcggtacattatgaaccttaacgtcaccttcctggtcaacagcgctgctcatatatatggaaagagaccttatgacaagaagatcctaccatctcaaaacatagctgtgtccattgcaacctttggggaaggtttccataattatcatcatgtatttccatgggattatcgcgcagctgaacttggaaataacagtttgaatttccctacgaaatttattgatttctttgcgtggatcggatgggcgtatgacctaaagactgtttcgaaagaaatgataaaacaaaggtcaaaaagaactggtgatggaactaatctatgggggttagaagatgtggataccccggaggatttaaaaaatacaaaaggcgaataggcaaacccttaaactcaaacagtgaggtttaatgtgatatttagaattagaattaatttatttgaaattaaatgaaggttttggataactgtttttaataataaaaatagtttttcgattaaattccttagattattttaaaggaaatgtataaggtactcgcgtggttagcaacccagcagtccctgtttatctgtttttatgaatttattctatgaatgtagatgtcgcatgaaattttaaaatgttgcatttgtataattttacttatgaataaataaatttatttttaaaaaaaaaaaaaaaaaaaaaaaaaaaaaaaaaa

SEQ ID NO: 41 - protein, 338 aa, Spodoptera littoralis
  PEPTIDE 1..338: note = delta 11 desaturase
MAQCVQTTTILEQKEEKTVTLLVPQAGKRKFEIVYFNIITFAYWHIAGLYGLYLCFTSTKWATVLFSFFLFVVAEVGVTAGSHRLWSHKTYKAKLPLQILLMVMNSLAFQNTVIDWVRDHRLHHKYSDTDADPHNASRGFFYSHVGWLLVRKHPDVKKRGKEIDISDIYNNPVLRFQKKYAIPFIGAVCFVLPTLIPVYGWGETWTNAWHVAMLRYIMNLNVTFLVNSAAHIYGKRPYDKKILPSQNIAVSIATFGEGFHNYHHVFPWDYRAAELGNNSLNFPTKFIDFFAWIGWAYDLKTVSKEMIKQRSKRTGDGTNLWGLEDVDTPEDLKNTKGE

SEQ ID NO: 42 - DNA, 1011 bp, Agrotis segetum
  source 1..1011: mol_type = other DNA
  CDS 1..1011: note = delta 11 desaturase
atggctcaaggtgtccaaacaactacgatattgagggaggaagagccgtcattgactttcgtggtacctcaagaaccgagaaagtatcaaatcgtgtacccaaaccttatcacatttgggtactggcatatagctggtttatacgggctatatttgtgctttacttcggcaaaatggcaaacaattttattcagtttcatgctcgttgtgttagcagagttgggaataacagccggcgctcacaggttatgggcccacaaaacatataaagcgaagcttcccttacaaattatcctgatgatactgaactccattgccttccaaaattccgccattgattgggtgagggaccaccgtctccatcataagtacagtgacactgatgcagaccctcacaatgctactcgtggtttcttctattctcatgttggatggttgctcgtaagaaaacatccagaagtcaagagacgtggaaaggaacttgacatgtctgatatttacaacaatccagtgctgagatttcaaaagaagtatgctatacccttcatcggggcaatgtgcttcggattaccaacttttatccctgtttacttctggggagaaacctggagtaatgcttggcatatcaccatgcttcggtacatcctcaacctaaacattactttcctggtcaacagtgctgctcatatctggggatacaaaccttatgacatcaaaatattgcctgcccaaaatatagcagtttccatagtaaccggcggcgaagtttccataactaccaccacgttttttccttgggattatcgtgcagcagaattggggaacaattatcttaatttgacgactaagttcatagatttcttcgcttggatcggatgggcttacgatcttaagacggtgtccagtgatgttataaaaagtaaggcggaaagaactggtgatgggacgaatctttggggtttagaagacaaaggtgaagaagattttttgaaaatctggaaagacaattaa

SEQ ID NO: 43 - protein, 336 aa, Agrotis segetum
MAQGVQTTTILREEEPSLTFVVPQEPRKYQIVYPNLITFGYWHIAGLYGLYLCFTSAKWQTILFSFMLVVLAELGITAGAHRLWAHKTYKAKLPLQIILMILNSIAFQNSAIDWVRDHRLHHKYSDTDADPHNATRGFFYSHVGWLLVRKHPEVKRRGKELDMSDIYNNPVLRFQKKYAIPFIGAMCFGLPTFIPVYFWGETWSNAWHITMLRYILNLNITFLVNSAAHIWGYKPYDIKILPAQNIAVSIVTGGEVSITTTTFFPWDYRAAELGNNYLNLTTKFIDFFAWIGWAYDLKTVSSDVIKSKAERTGDGTNLWGLEDKGEEDFLKIWKDN

SEQ ID NO: 44 - DNA, 1050 bp, Trichoplusia ni
  source 1..1050: mol_type = other DNA
  CDS 1..1050: note = delta 11 desaturase
atggctgtgatggctcaaacagtacaagaaacggctacagtgttggaagaggaagctcgcacagtgactcttgtggctccaaagacaacgccaaggaaatataaatatatatacaccaactttcttacattttcatatgcgcatttagctgcattatacggactttatttgtgcttcacctctgcgaaatgggaaacattgctattctctttcgtactcttccacatgtcaaatataggcatcaccgcaggggctcaccgactctggactcacaagactttcaaagccaaattgcctttggaaattgtcctcatgatattcaactctttagcctttcaaaacacggctattacatgggctagagaacatcggctacatcacaaatacagcgatactgatgctgatccccacaatgcgtcaagagggttcttctactcgcatgttggctggctattagtaaaaaaacatcccgatgtcctgaaatatggaaaaactatagacatgtcggatgtatacaataatcctgtgttaaaatttcagaaaaagtacgcagtacccttaattggaacagtttgttttgctcttccaactttgattccagtctactgttggggcgaatcgtggaacaacgcttggcacatagccttatttcgatacatattcaatcttaacgtgactttcctagtcaacagtgctgcgcatatctgggggaataagccttatgataaaagcatcttgcccgctcaaaacctgctggtttccttcctagcaagtggagaaggcttccataattaccatcacgtctttccatgggattaccgcacagcagaattagggaataacttcctgaatttgacgacgctgttcattgatttttgtgcctggtttggatgggcttatgacttgaagtctgtatcagaggatattataaaacagagagctaaacgaacaggtgacggttcttcaggggtcatttggggatgggacgacaaagacatggaccgcgatataaaatctaaagctaacattttttatgctaaaaaggaatga

SEQ ID NO: 45 - protein, 349 aa, Trichoplusia ni
MAVMAQTVQETATVLEEEARTVTLVAPKTTPRKYKYIYTNFLTFSYAHLAALYGLYLCFTSAKWETLLFSFVLFHMSNIGITAGAHRLWTHKTFKAKLPLEIVLMIFNSLAFQNTAITWAREHRLHHKYSDTDADPHNASRGFFYSHVGWLLVKKHPDVLKYGKTIDMSDVYNNPVLKFQKKYAVPLIGTVCFALPTLIPVYCWGESWNNAWHIALFRYIFNLNVTFLVNSAAHIWGNKPYDKSILPAQNLLVSFLASGEGFHNYHHVFPWDYRTAELGNNFLNLTTLFIDFCAWFGWAYDLKSVSEDIIKQRAKRTGDGSSGVIWGWDDKDMDRDIKSKANIFYAKKE

SEQ ID NO: 46 - DNA, 2235 bp, Saccharomyces cerevisiae
  source 1..2235: mol_type = other DNA
  CDS 1..2235: note = FAA2
atggccgctccagattatgcacttaccgatttaattgaatcggatcctcgtttcgaaagtttgaagacaagattagccggttacaccaaaggctctgatgaatatattgaagagctatactctcaattaccactgaccagctaccccaggtacaaaacatttttaaagaaacaggcggttgccatttcgaatccggataatgaagctggttttagctcgatttataggagttctctttcttctgaaaatctagtgagctgtgtggataaaaacttaagaactgcatacgatcacttcatgttttctgcaaggagatggcctcaacgtgactgtttaggttcaaggccaattgataaagccacaggcacctgggaggaaacattccgtttcgagtcgtactccacggtatctaaaagatgtcataatatcggaagtggtatattgtctttggtaaacacgaaaaggaaacgtcctttggaagccaatgattttgttgttgctatcttatcacacaacaaccctgaatggatcctaacagatttggcctgtcaggcctattctctaactaacacggctttgtacgaaacattaggtccaaacacctccgagtacatattgaatttaaccgaggcccccattctgatttttgcaaaatcaaatatgtatcatgtattgaagatggtgcctgatatgaaatttgttaatactttggtttgtatggatgaattaactcatgacgagctccgtatgctaaatgaatcgttgctacccgttaagtgcaactctctcaatgaaaaaatcacatttttttcattggagcaggtagaacaagttggttgctttaacaaaattcctgcaattccacctaccccagattccttgtatactatttcgtttacttctggtactacaggtttacctaaaggtgtggaaatgtctcacagaaacattgcgtctgggatagcatttgctttttctaccttcagaataccgccagataaaagaaaccaacagttatatgatatgtgttttttgccattggctcatatttttgaaagaatggttattgcgtatgatctagccatcgggtttggaataggcttcttacataaaccagacccaactgtattggtagaggatttgaagattttgaaaccttacgcggttgccctggttcctagaatattaacacggtttgaagccggtataaaaaatgctttggataaatcgactgtccagaggaacgtagcaaatactatattggattctaaatcggccagatttaccgcaagaggtggtccagataaatcgattatgaattttctagtttatcatcgcgtattgattgataaaatcagagactctttaggtttgtccaataactcgtttataattaccggatcagctcccatatctaaagataccttactatttttaagaagcgccttggatattggtataagacagggctacggcttaactgaaacttttgctggtgtctgtttaagcgaaccgtttgaaaaagatgtcggatcttgtggtgccataggtatttctgcagaatgtagattgaagtctgttccagaaatgggttaccatgccgacaaggatttaaaaggtgaactgcaaattcgtggcccacaggtttttgaaagatattttaaaaatccgaatgaaacttcaaaagccgttgaccaagatggttggttttccacgggagatgttgcatttatcgatgcaaaaggtcgcatcagcgtcattgatcgagtcaagaactttttcaagctagcacatggtgaatatattgctccagagaaaatcgaaaatatttatttatcatcatgcccctatatcacgcaaatatttgtctttggagatcctttgaagacatttttagttggcatcgttggtgttgatgttgatgcagcgcaaccgattttagctgcaaagcacccagaggtgaaaacgtggactaaggaagtgctagtagaaaacttaaatcgtaataaaaagctaaggaaggaatttttaaacaaaattaataaatgcatcgatgggctacaaggatttgaaaaattgcacaacatcaaagtcggacttgagcctttgactctcgaggatgatgttgtgacgccaacttttaaaataaagcgtgccaaagcatcaaaattcttcaaagatacattagaccaactatacgccgaaggttcactagtcaagacagaaaagctttag

SEQ ID NO: 47 - protein, 744 aa, Saccharomyces cerevisiae
MAAPDYALTDLIESDPRFESLKTRLAGYTKGSDEYIEELYSQLPLTSYPRYKTFLKKQAVAISNPDNEAGFSSIYRSSLSSENLVSCVDKNLRTAYDHFMFSARRWPQRDCLGSRPIDKATGTWEETFRFESYSTVSKRCHNIGSGILSLVNTKRKRPLEANDFVVAILSHNNPEWILTDLACQAYSLTNTALYETLGPNTSEYILNLTEAPILIFAKSNMYHVLKMVPDMKFVNTLVCMDELTHDELRMLNESLLPVKCNSLNEKITFFSLEQVEQVGCFNKIPAIPPTPDSLYTISFTSGTTGLPKGVEMSHRNIASGIAFAFSTFRIPPDKRNQQLYDMCFLPLAHIFERMVIAYDLAIGFGIGFLHKPDPTVLVEDLKILKPYAVALVPRILTRFEAGIKNALDKSTVQRNVANTILDSKSARFTARGGPDKSIMNFLVYHRVLIDKIRDSLGLSNNSFIITGSAPISKDTLLFLRSALDIGIRQGYGLTETFAGVCLSEPFEKDVGSCGAIGISAECRLKSVPEMGYHADKDLKGELQIRGPQVFERYFKNPNETSKAVDQDGWFSTGDVAFIDAKGRISVIDRVKNFFKLAHGEYIAPEKIENIYLSSCPYITQIFVFGDPLKTFLVGIVGVDVDAAQPILAAKHPEVKTWTKEVLVENLNRNKKLRKEFLNKINKCIDGLQGFEKLHNIKVGLEPLTLEDDVVTPTFKIKRAKASKFFKDTLDQLYAEGSLVKTEKL

SEQ ID NO: 48 - DNA, 25 bp, synthetic construct
  misc_feature 1..25: note = Artificial sequence
  primer_bind 1..25: note = PR-14126 (Ased11_U1_fw)
  source 1..25: mol_type = other DNA
  modified_base 9: mod_base = OTHER; note = uracil
agtgcaggtaaaacaatggctcaag

SEQ ID NO: 49 - DNA, 22 bp, synthetic construct
  misc_feature 1..22: note = Artificial sequence
  primer_bind 1..22: note = PR-14127 (Ased11_U1_rev)
  source 1..22: mol_type = other DNA
  modified_base 8: mod_base = OTHER; note = uracil
cgtgcgatttagttgtccttcc

SEQ ID NO: 50 - DNA, 25 bp, synthetic construct
  misc_feature 1..25: note = Artificial sequence
  primer_bind 1..25: note = PR-14128 (Sld11_U1_fw)
  source 1..25: mol_type = other DNA
  modified_base 9: mod_base = OTHER; note = uracil
agtgcaggtaaaacaatggctcaat

SEQ ID NO: 51 - DNA, 21 bp, synthetic construct
  misc_feature 1..21: note = Artificial sequence
  primer_bind 1..21: note = PR-14129 (Sld11_U1_rev)
  source 1..21: mol_type = other DNA
  modified_base 8: mod_base = OTHER; note = uracil
cgtgcgattcattcaccctta

SEQ ID NO: 52 - DNA, 27 bp, synthetic construct
  misc_feature 1..27: note = Artificial sequence
  primer_bind 1..27: note = PR-14130 (Tnd11_U1_fw)
  source 1..27: mol_type = other DNA
  modified_base 9: mod_base = OTHER; note = uracil
agtgcaggtaaaacaatggctgttatg

SEQ ID NO: 53 - DNA, 30 bp, synthetic construct
  misc_feature 1..30: note = Artificial sequence
  primer_bind 1..30: note = PR-14131 (Tnd11_U1_rev)
  source 1..30: mol_type = other DNA
  modified_base 8: mod_base = OTHER; note = uracil
cgtgcgattcattctttcttagcgtagaaa

(19) United States
(12) Patent Application Publication
(10) Pub. No.: US 2024/0076329 A1
(43) Pub. Date: Mar. 7, 2024

(54) METHOD FOR PRODUCTION OF MOTH PHEROMONES IN YEAST

(71) Applicant: Danmarks Tekniske Universitet, Kgs. Lyngby (DK)

(72) Inventors: Irina Borodina, Nivå (DK); Carina Holkenbrink, Kongens Lyngby (DK); Marie Inger Dam, Birkerød (DK); Christer Löfstedt, Lund (SE); Baojian Ding, Södra Sandby (SE); Hong-Lei Wang, Lund (SE)

(21) Appl. No.: 18/144,503

(22) Filed: May 8, 2023

Related U.S. Application Data
(63) Continuation of application No. 15/580,320, filed on Dec. 7, 2017, now Pat. No. 11,685,768.

(30) Foreign Application Priority Data
Jun. 26, 2015 (EP) 15174099.0

(51) Int. Cl. C07K 14/435; C12N 1/16; C12P 7/04; C12P 7/24; C12P 7/6436

(52) U.S. Cl. CPC C07K 14/43563; C12N 1/16; C12P 7/04; C12P 7/24; C12P 7/6436; C12Y 102/01084; C12Y 114/19005; C12Y 203/01086; Y02P 20/582

(57) ABSTRACT

The present disclosure relates to methods for production of (Z)-11-hexadecen-1-ol in a yeast cell. Also disclosed are methods for production of (Z)-11-hexadecenal in a yeast cell. Also disclosed are methods for production of (Z)-11-hexadecen-1-yl acetate in a yeast cell. The disclosure also provides for nucleic acid constructs and yeast cells useful for performing the present methods, as well as to pheromone compositions.

### CROSS REFERENCE TO RELATED APPLICATIONS

This application is a continuation of Ser. No. 15/580,320 filed Dec. 7, 2017, which is a U.S. National Stage application under 35 U.S.C. § 371 of PCT/EP2016/064651 filed Jun. 24, 2016, which depends from and claims priority to European application number 15174099.0 filed Jun. 26, 2015, the entire contents of each of which are incorporated herein by reference.

### SEQUENCE LISTING

The instant application contains a Sequence Listing which has been submitted in .xml format and is hereby incorporated by reference in its entirety. Said .xml copy, created on Nov. 16, 2023, is named 2023-11-16 16DMTU-H062603NA Sequence Listing_corrected XML.xml and is 120,455 bytes in size.

### FIELD OF INVENTION

Herein are disclosed methods for production of (Z)-11-hexadecen-1-ol in a yeast cell. Also disclosed are methods for production of (Z)-11-hexadecenal in a yeast cell. Also disclosed are methods for production of (Z)-11-hexadecen-1-yl acetate in a yeast cell. The disclosure also provides for nucleic acid constructs and yeast cells useful for performing the present methods, as well as to pheromone compositions.

### BACKGROUND OF INVENTION

Integrated Pest Management (IPM) is expected to play a major role for both increasing the crop yield and for minimizing environmental impact and enabling organic food production. IPM employs alternative pest control methods, such as mating disruption using pheromones, mass trapping using pheromones, beneficial insects, etc.

Pheromones constitute a group of diverse chemicals that insects (like other organisms) use to communicate with individuals of the same species in various contexts, including mate attraction, alarm, trail marking and aggregation. Insect pheromones associated with long-range mate finding are already used in agriculture and forestry applications for monitoring and control of pests, as a safe and environmentally friendly alternative to pesticides.

Pheromones for pest control can be divided up in four categories: sex pheromones, aggregation pheromones, oviposition-deterring pheromones and alarm pheromones. Sex pheromones are the largest product segment accounting for 64.8% of the global IPM pheromones market revenues in 2013. Sex pheromones are widely used for pest monitoring and for pest control by mating disruption (e.g., in apple and peach orchards, in forests) and mass trapping (e.g., protection of tomatoes in green houses). Mating disruption occurs when pheromones are released in small amounts into the air and prevent males from locating females, which leads to the eventual cessation of breeding and collapse of the insect infestation. Aggregation pheromones are used to attract both male and female pests and hence are applied for mass trapping. Mass trapping helps maintain population densities below economic damage threshold when insects are caught using attractive baits.

Application of insect pheromones for pest control became possible only after industrial-scale synthesis of pheromones started at the end of the 1980s. Nevertheless, the prices for chemically synthesized pheromones remain high and present a major barrier for expanding their usage in agriculture and forestry. Another drawback with the chemical production of pheromones is the requirement for toxic chemicals to be used as precursors, catalyzers and solvents, and large amounts of organic waste generated during the purification.

Pheromones are currently produced by complex chemical synthesis-based processes, which make the products prohibitively expensive for widespread use in many of the potential applications in agriculture and forestry.

There are several advantages to biological production methods as compared to chemical production methods. First, all the reactions are carried out by engineered cells at ambient temperatures in fermentation tanks instead of multiple chemical reaction steps requiring different precursors, catalyzers and conditions (often high temperatures and pressures). Moreover, the engineered cells use cheap renewable materials, such as sugars or plant oils, instead of using multiple expensive specialty chemicals as precursors. While chemical reactions often suffer from low specificity, and thus require purification of intermediate compounds and extensive purification of the final product, biological reactions carried out by enzymes are typically very specific and formation of by-products is limited, thereby reducing the usage of organic solvents and other toxic chemicals for purification. Moreover, specific stereo-chemistry, which is often important for pheromone activity, can be very difficult to achieve by chemical methods, while enzymatic methods can take advantage of enzymes specific for one of the cis- or trans-isomers.

Hence, there is a need for biological processes for production of insect pheromones. In addition to lower cost benefits, fermentation processes are inherently less hazardous and more environmentally friendly than chemical synthesis.

### SUMMARY OF INVENTION

The invention is as defined in the claims.

Herein provided is a method for production of a moth pheromone in a yeast cell, said method comprising the steps of:
i) providing a yeast cell capable of synthesising hexadecanoyl-CoA, said yeast cell expressing:
a Δ11-desaturase selected from the group consisting of a Δ11-desaturase comprising SEQ ID NO: 2, a Δ11-desaturase comprising SEQ ID NO: 41, a Δ11-desaturase comprising SEQ ID NO: 43, and a Δ11-desaturase comprising SEQ ID NO: 45 or a Δ11-desaturase variant having at least 85% sequence identity to the polypeptide of SEQ ID NO: 2, SEQ ID NO: 41, SEQ ID NO: 43, or SEQ ID NO: 45, said Δ11-desaturase variant having Δ11-desaturase activity, and
an alcohol-forming fatty acyl-CoA reductase (FAR) selected from the group consisting of a FAR comprising SEQ ID NO: 8, a FAR comprising SEQ ID NO: 16, and a FAR comprising SEQ ID NO: 12 or a FAR variant having at least 85% sequence identity to the polypeptide of SEQ ID NO: 8, or SEQ ID NO: 12 or a FAR variant having at least 99% sequence identity to the polypeptide of SEQ ID NO: 16, said FAR variant having alcohol-forming fatty acyl-CoA reductase activity;
ii) expressing said Δ11-desaturase and said FAR from said yeast cell; and
iii) incubating said yeast cell in a medium, whereby
   the Δ11-desaturase is capable of converting at least part of said hexadecanoyl-CoA
to (Z)11-hexadecenoyl-CoA; and
said FAR is capable of converting at least part of said (Z)11-hexadecenoyl-CoA to (Z)-11-hexadecen-1-ol,
   thereby obtaining a moth pheromone comprising (Z)-11-hexadecen-1-ol with a titre of at least 1 mg/L, wherein the titre is measured in a culture medium after culturing the yeast cell.

In another aspect, the invention relates to a method for production of (Z)-11-hexadecenal in a yeast cell, said method comprising the steps of:
i) providing a yeast cell capable of synthesising hexadecanoyl-CoA, said yeast cell further capable of expressing:
   the Amyelois transitella Δ11-desaturase (Atr_Δ11; SEQ ID NO: 2), the Spodoptera littoralis Δ11-desaturase (SI_Δ11; SEQ ID NO: 41), the Agrotis segetum Δ11-desaturase (As_Δ11; SEQ ID NO: 43), the Trichoplusia ni Δ11-desaturase (Tni_Δ11; SEQ ID NO: 45) or a variant thereof having at least 65% homology, such as at least 70% homology, such as at least 71% homology, such as at least 72%, such as at least 73%, such as at least 74%, such as at least 75%, such as at least 80%, such as at least 85%, such as at least 90%, such as at least 95%, such as 100% homology to Atr_Δ11 (SEQ ID NO: 2), SI_Δ11 (SEQ ID NO: 41), As_Δ11 (SEQ ID NO: 43), or Tni_Δ11 (SEQ ID NO: 45), and
   an aldehyde-forming fatty acyl-CoA reductase (FAR′);
ii) expressing said Δ11-desaturase and said FAR′ from said yeast cell; and
iii) incubating said yeast cell in a medium,
   whereby
the Δ11-desaturase converts at least part of said hexadecanoyl-CoA to (Z)11-hexadecenoyl-CoA; and
said FAR′ converts at least part of said (Z)-11-hexadecenoyl-CoA to (Z)-11-hexadecenal,
thereby obtaining (Z)-11-hexadecenal.

In yet another aspect, the invention relates to (Z)-11-hexadecen-1-ol obtainable by the methods disclosed herein.

In yet another aspect, the invention relates to (Z)-11-hexadecenal obtainable by the methods disclosed herein.

In yet another aspect, the invention relates to (Z)-11-hexadecen-1-yl acetate obtainable by the methods disclosed herein.

In yet another aspect, the invention relates to a pheromone composition comprising (Z)-11-hexadecenol, (Z)-11-hexadecenal or (Z)-11-hexadecen-1-yl acetate obtainable by the methods disclosed herein.

In yet another aspect, the invention relates to the use of a pheromone composition as defined herein for monitoring the presence of pest and/or disrupting the mating of pest.

In yet another aspect, the invention relates to a method of monitoring the presence of pest or disrupting the mating of pest, said method comprising the steps of:
i) producing (Z)-11-hexadecenol and optionally (Z)-11-hexadecenal and/or (Z)-11-hexadecen-1-yl acetate by the present methods,
ii) formulating said (Z)-11-hexadecenol and optionally (Z)-11-hexadecenal and/or (Z)-11-hexadecen-1-yl acetate thus obtained into a pheromone composition, and
iii) employing said pheromone composition as an integrated pest management composition.

In yet another aspect, the invention relates to a nucleic acid construct comprising one or more of:
a nucleic acid sequence identical to or having at least 80% homology to SEQ ID NO: 1, SEQ ID NO: 40, SEQ ID NO: 42 or SEQ ID NO: 44; and/or
a nucleic acid sequence identical to or having at least 90% homology to SEQ ID NO: 7, SEQ ID NO: 11 or SEQ ID NO: 15; and/or
a nucleic acid sequence identical to or having at least 65% homology to SEQ ID NO: 34 or at least 65% homology to SEQ ID NO: 36; and/or
a nucleic acid sequence identical to or having at least 65% homology to SEQ ID NO: 38.

In het another aspect, the invention relates to yeast cell capable of synthesising hexadecanoyl-CoA, said yeast cell further capable of expressing:
a Δ11-desaturase selected from the group consisting of a Δ11-desaturase comprising SEQ ID NO: 2, a Δ11-desaturase comprising SEQ ID NO: 41, a Δ11-desaturase comprising SEQ ID NO: 43, and a Δ11-desaturase comprising SEQ ID NO: 45 or a Δ11-desaturase variant having at least 85% sequence identity to the polypeptide of SEQ ID NO: 2, SEQ ID NO: 41, SEQ ID NO: 43, or SEQ ID NO: 45, said Δ11-desaturase variant having Δ11-desaturase activity, and
an alcohol-forming fatty acyl-CoA reductase (FAR) selected from the group consisting of a FAR comprising SEQ ID NO: 8, a FAR comprising SEQ ID NO: 16, and a FAR comprising SEQ ID NO: 12 or a FAR variant having at least 85% sequence identity to the polypeptide of SEQ ID NO: 8, or SEQ ID NO: 12 or a FAR variant having at least 99% sequence identity to the polypeptide of SEQ ID NO: 16, said FAR variant having alcohol-forming fatty acyl-CoA reductase activity.

In yet another aspect, the invention relates to a yeast cell comprising one or more of:
a nucleic acid sequence identical to or having at least 80% homology to SEQ ID NO: 1, SEQ ID NO: 40, SEQ ID NO: 42 or SEQ ID NO: 44; and/or
a nucleic acid sequence identical to or having at least 90% homology to SEQ ID NO: 7, SEQ ID NO: 11 or SEQ ID NO: 15; and/or
a nucleic acid sequence identical to or having at least 65% homology to SEQ ID NO: 34 or at least 65% homology to SEQ ID NO: 36; and/or
a nucleic acid sequence identical to or having at least 65% homology to SEQ ID NO: 38.

In yet another aspect, the invention relates to a yeast cell capable of synthesising hexadecanoyl-CoA, said yeast cell further capable of expressing:
the Amyelois transitella Δ11-desaturase (Atr_Δ11; SEQ ID NO: 2), the Spodoptera littoralis Δ11-desaturase (SI_Δ11; SEQ ID NO: 41), the Agrotis segetum Δ11-desaturase (As_Δ11; SEQ ID NO: 43), the Trichoplusia ni Δ11-desaturase (Tni_Δ11; SEQ ID NO: 45) or a variant thereof having at least 65% homology, such as at least 70% homology, such as at least 71% homology, such as at least 72%, such as at least 73%, such as at least 74%, such as at least 75%, such as at least 80%, such as at least 85%, such as at least 90%, such as at least 95%, such as 100% homology to Atr_Δ11 (SEQ ID NO: 2), SI_Δ11 (SEQ ID NO: 41), As_Δ11 (SEQ ID NO: 43), or Tni_Δ11 (SEQ ID NO: 45), and
an alcohol-forming fatty acyl-CoA reductase (FAR) selected from the group consisting of Har_FAR (SEQ ID NO: 8), Hs_FAR (SEQ ID NO: 16), and Has_FAR (SEQ ID NO: 12), or a variant thereof having at least 75% homology, such as at least 80%, such as at least 85%, such as at least 90%, such as at least 95%, such as 100% homology to Har_FAR (SEQ ID NO: 8), Hs_FAR (SEQ ID NO: 16), or Has_FAR (SEQ ID NO: 12).

In yet another aspect, the invention relates to a yeast cell capable of synthesising hexadecanoyl-CoA, said yeast cell further capable of expressing:
the Amyelois transitella Δ11-desaturase (Atr_Δ11; SEQ ID NO: 2), the Spodoptera littoralis Δ11-desaturase (SI_Δ11; SEQ ID NO: 41), the Agrotis segetum Δ11-desaturase (As_Δ11; SEQ ID NO: 43), the Trichoplusia ni Δ11-desaturase (Tni_Δ11; SEQ ID NO: 45) or a variant thereof having at least 65% homology, such as at least 70% homology, such as at least 71% homology, such as at least 72%, such as at least 73%, such as at least 74%, such as at least 75%, such as at least 80%, such as at least 85%, such as at least 90%, such as at least 95%, such as 100% homology to Atr_Δ11 (SEQ ID NO: 2), SI_Δ11 (SEQ ID NO: 41), As_Δ11 (SEQ ID NO: 43), or Tni_Δ11 (SEQ ID NO: 45), and
a fatty acyl-CoA reductase (FAR).

In yet another aspect, the invention relates to a kit of parts comprising a yeast cell and/or a nucleic acid construct as disclosed herein, and instructions for use.

### DESCRIPTION OF DRAWINGS

FIG. 1. Heterologous (Z)-11-hexadecen-1-ol pathway. group FAA: Fatty acyl CoA synthetase, Δ11 FAD: Δ11-fatty-acyl CoA desaturase, FAR: alcohol-forming fatty acyl-CoA reductase; FAR′: aldehyde-forming fatty acyl-CoA reductase, AcT: acetyltransferase; 1: palmitic acid, 2: hexadecanoyl-CoA, 3: (Z)-11-hexadecenoyl-CoA, 4: (Z)-11-hexadecen-1-ol, 5: (Z)-11-hexadecen-1-yl-acetate, 6: (Z)-11-hexadecenal.

FIG. 2. (Z)-11-hexadecen-1-ol production during fed-batch fermentation. X-axis shows (Z)-11-hexadecen-1-ol titre in mg/L, Y-axis shows fermentation time in hours. (A) Titres obtained with integration of a single gene copy (strain ST3705). (B) Titres obtained with integration of multiple gene copies (strain ST5262).

FIG. 3. Vector map of plasmid pCfB3465. The vector encodes expressions cassettes for the Atrd11, Hs_FAR, and URA3 gene. The expression is driven from Y. lipolytica native promoters (Pr). The expression cassettes are flanked by genomic DNA sequences (IntB_up and IntB_down) of 500 bp allowing site-specific integration into the Y. lipolytica genome.

### DEFINITIONS

Biopesticide: the term ‘biopesticide’ is a contraction of ‘biological pesticide’ and refers to several types of pest management intervention: through predatory, parasitic, or chemical relationships. In the EU, biopesticides have been defined as “a form of pesticide based on micro-organisms or natural products”. In the US, they are defined by the EPA as “including naturally occurring substances that control pests (biochemical pesticides), microorganisms that control pests (microbial pesticides), and pesticidal substances produced by plants containing added genetic material (plant-incorporated protectants) or PIPs”. The present disclosure relates more particularly to biopesticides comprising natural products or naturally occurring substances. They are typically created by growing and concentrating naturally occurring organisms and/or their metabolites including bacteria and other microbes, fungi, nematodes, proteins, etc. They are often considered to be important components of integrated pest management (IPM) programmes, and have received much practical attention as substitutes to synthetic chemical plant protection products (PPPs). The Manual of Biocontrol Agents (2009: formerly the Biopesticide Manual) gives a review of the available biological insecticide (and other biology-based control) products.

Pest: as used herein, the term ‘pest’ shall refer to an organism, in particular an animal, detrimental to humans or human concerns, in particular in the context of agriculture or livestock production. A pest is any living organism which is invasive or prolific, detrimental, troublesome, noxious, destructive, a nuisance to either plants or animals, human or human concerns, livestock, human structures, wild ecosystems etc. The term often overlaps with the related terms vermin, weed, plant and animal parasites and pathogens. It is possible for an organism to be a pest in one setting but beneficial, domesticated or acceptable in another.

### DETAILED DESCRIPTION OF THE INVENTION

The present disclosure relates to methods for producing moth pheromones in a yeast cell, in particular (Z)-11-hexadecen-1-ol, (Z)-11-hexadecenal, and (Z)-11-hexadecen-1-yl acetate, which are components of the insect sex pheromone in large cabbage-heart caterpillar Crocidolomia binotalis, diamond back moth Plutella xylostella, cabbage moth Mamestra brassicae, corn stalk borer Sesamia nonagrioides, artichoke plume moth Platyptilia carduidactyla, cotton boolworm Helicoverpa armigera, stem borer Chilo suppressalis and other moths. The inventors have been able to obtain surprisingly high titres of (Z)-11-hexadecen-1-ol using the methods described herein.

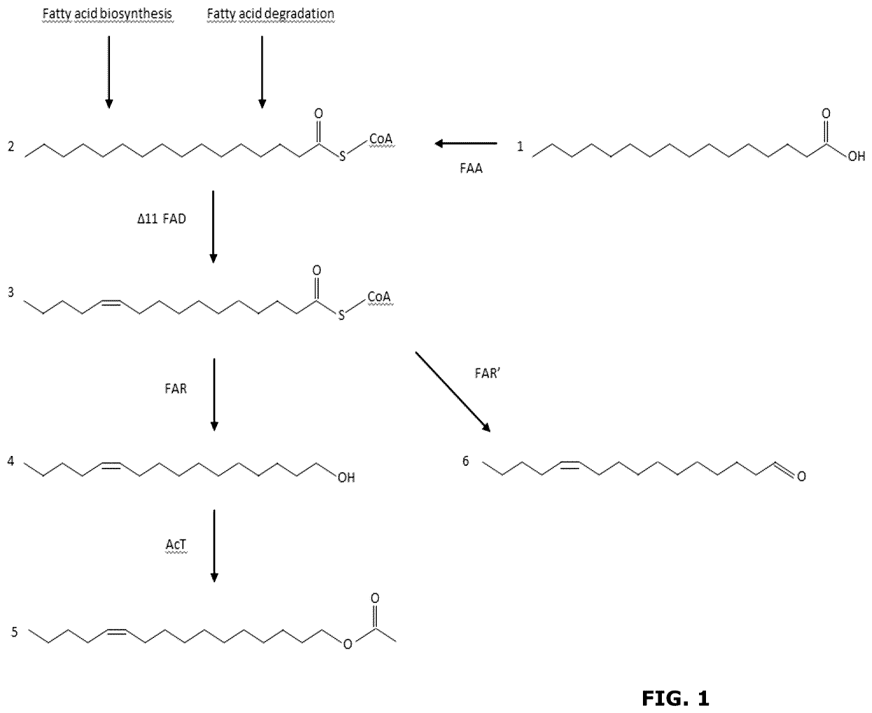

Production of (Z)-11-hexadecen-1-ol Herein are disclosed methods for producing (Z)-11-hexadecen-1-ol from a yeast cell. The inventors have designed a heterologous pathway which is outlined in FIG. 1 by way of example. Hexadecanoyl-CoA is a native fatty acid intermediate in fatty acid metabolism. Hexadecanoyl-CoA is converted to (Z)-11-hexadecenoyl-CoA by a Δ11-fatty acyl desaturase (Δ11 FAD), which in turn is converted to (Z)-11-hexadecen-1-ol by an alcohol-forming fatty acyl-CoA reductase (FAR).

In a first aspect, the present disclosure thus relates to a method for production of (Z)-11-hexadecen-1-ol in a yeast cell, said method comprising the steps of:
i) providing a yeast cell capable of synthesising hexadecanoyl-CoA, said yeast cell further capable of expressing:
   the Amyelois transitella Δ11-desaturase (Atr_Δ11; SEQ ID NO: 2), the Spodoptera littoralis Δ11-desaturase (SI_Δ11; SEQ ID NO: 41), the Agrotis segetum Δ11-desaturase (As_Δ11; SEQ ID NO: 43), the Trichoplusia ni Δ11-desaturase (Tni_Δ11; SEQ ID NO: 45) or a variant thereof having at least 65% homology, such as at least 70% homology, such as at least 71% homology, such as at least 72%, such as at least 73%, such as at least 74%, such as at least 75%, such as at least 80%, such as at least 85%, such as at least 90%, such as at least 95%, such as 100% homology to Atr_Δ11 (SEQ ID NO: 2), SI_Δ11 (SEQ ID NO: 41), As_Δ11 (SEQ ID NO: 43), or Tni_Δ11 (SEQ ID NO: 45), and
   an alcohol-forming fatty acyl-CoA reductase (FAR) selected from the group consisting of Har_FAR (SEQ ID NO: 8), Hs_FAR (SEQ ID NO: 16), and Has_FAR (SEQ ID NO: 12), or a variant thereof having at least 75% homology, such as at least 80%, such as at least 85%, such as at least 90%, such as at least 95%, such as 100% homology to Har_FAR (SEQ ID NO: 8), Hs_FAR (SEQ ID NO: 16), or Has_FAR (SEQ ID NO: 12);
ii) expressing said Δ11-desaturase and said FAR from said yeast cell; and
iii) incubating said yeast cell in a medium,
   whereby
the Δ11-desaturase is capable of converting at least part of said hexadecanoyl-CoA to (Z)11-hexadecenoyl-CoA; and
said FAR is capable of converting at least part of said (Z)11-hexadecenoyl-CoA to (Z)-11-hexadecenol,
   thereby obtaining (Z)-11-hexadecen-1-ol with a titre of at least 0.2 mg/L.

Accordingly, the present disclosure provides a method for production of (Z)-11-hexadecen-1-ol in a yeast cell, said method comprising the steps of:
i) providing a yeast cell capable of synthesising hexadecanoyl-CoA, said yeast cell further capable of expressing:
   a Δ11-desaturase selected from the group consisting of the Amyelois transitella Δ11-desaturase (Atr_Δ11; SEQ ID NO: 2), the Spodoptera littoralis Δ11-desaturase (SI_Δ11; SEQ ID NO: 41), the Agrotis segetum Δ11-desaturase (As_Δ11; SEQ ID NO: 43) and the Trichoplusia ni Δ11-desaturase (Tni_Δ11; SEQ ID NO: 45) or a variant thereof having at least 65% homology, such as at least 70% homology, such as at least 71% homology, such as at least 72%, such as at least 73%, such as at least 74%, such as at least 75%, such as at least 80%, such as at least 85%, such as at least 90%, such as at least 95%, such as 100% homology to Atr_Δ11 (SEQ ID NO: 2), SI_Δ11 (SEQ ID NO: 41), As_Δ11 (SEQ ID NO: 43), or Tni_Δ11 (SEQ ID NO: 45), and
   an alcohol-forming fatty acyl-CoA reductase (FAR) selected from the group consisting of Har_FAR (SEQ ID NO: 8), Hs_FAR (SEQ ID NO: 16), and Has_FAR (SEQ ID NO: 12), or a variant thereof having at least 75% homology, such as at least 80%, such as at least 85%, such as at least 90%, such as at least 95%, such as 100% homology to Har_FAR (SEQ ID NO: 8), Hs_FAR (SEQ ID NO: 16), or Has_FAR (SEQ ID NO: 12);
ii) expressing said Δ11-desaturase and said FAR from said yeast cell; and
iii) incubating said yeast cell in a medium,
   whereby
the Δ11-desaturase is capable of converting at least part of said hexadecanoyl-CoA to (Z)11-hexadecenoyl-CoA; and
said FAR is capable of converting at least part of said (Z)11-hexadecenoyl-CoA to (Z)-11-hexadecenol,
   thereby obtaining (Z)-11-hexadecen-1-ol with a titre of at least 0.2 mg/L.

Yeast Cell

In the first step of the present method, a yeast cell is provided capable of synthesising hexadecanoyl-CoA.

Any yeast cell capable of synthesising hexadecanoyl-CoA can be used for producing (Z)-11-hexadecen-1-ol as described herein.

In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In some embodiments, the genus of said yeast is Saccharomyces or Yarrowia.

The yeast cell may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell or a Yarrowia lipolytica cell.

Fatty Acyl-CoA Synthetase (FAA) (EC 2.3.1.86)

The terms ‘fatty acyl-CoA synthetase’, ‘fatty acyl-CoA synthase’ and ‘FAA’ will be used herein interchangeably.

Hexadecanoyl-CoA is a key intermediate in fatty acid biosynthesis, and it can also be synthesized as an intermediate of lipid degradation. Biosynthesis of hexadecanoyl-CoA can be enhanced by overexpression of genes involved in lipid biosynthesis, such as fatty acid synthases of type I or type II, and/or by overexpression of acetyl-CoA carboxylase, or by improving the supply of acetyl-CoA precursor. Hexadecanoyl-CoA can also be formed via fatty acyl-CoA synthetase from hexadecanoic acid (palmitic acid), which is either supplied in the broth or is synthesized by thioesterase intracellularly. FAA activity is normally present in organisms which are capable of metabolising fatty acids. It may be encoded by several, redundant enzymes. Thus in some embodiments, the yeast cell is further capable of expressing an FAA. The nucleic acid encoding said FAA activity in the yeast cell may be naturally present in the genome of said yeast cell, or it may be introduced by genetic engineering or genome editing. Thus in some embodiments the FAA activity is encoded by introduction of a heterologous nucleic acid in the yeast cell. The heterologous nucleic acid encoding said FAA may be codon-optimised, or may comprise features that can help improve the FAA activity. For example, the heterologous nucleic acid may be modified so as to encode a modified FAA. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the FAA activity can be performed by methods known in the art. The skilled person will recognise that such methods include, but are not limited to: cloning and homologous recombination-based methods. Cloning methods may involve the design and construction of a plasmid in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

In one embodiment, the FAA is Sc_FAA1 (SEQ ID NO: 35) or YI_FAA (SEQ ID NO: 37), or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to Sc_FAA1 (SEQ ID NO: 35) or YI_FAA (SEQ ID NO: 37). Thus in one embodiment, the yeast cell is a S. cerevisiae cell and the FAA is Sc_FAA1 (SEQ ID NO:35) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to Sc_FAA1 (SEQ ID NO: 35). In another embodiment, the yeast cell is a Y. lipolytica cell and the FAA is YI_FAA (SEQ ID NO: 37) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to YI_FAA (SEQ ID NO: 37). In another embodiment, the yeast cell is a S. cerevisiae cell and the FAA is YI_FAA (SEQ ID NO: 37) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to YI_FAA (SEQ ID NO: 37). In yet another embodiment, the yeast cell is a Y. lipolytica cell and the FAA is Sc_FAA1 (SEQ ID NO:35) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to Sc_FAA1 (SEQ ID NO: 35).

In some embodiments, the FAA is Sc_FAA2 (SEQ ID NO: 47).

Δ11-fatty acyl desaturase (Δ11 FAD) (EC 1.14.19.5)

In the present disclosure, the terms ‘Δ11-fatty acyl-CoA desaturase’, ‘Δ11-desaturase’ ‘Δ11-fatty acyl desaturase’ and ‘Δ11FAD’ will be used interchangeably and all refer to the EC enzyme having an EC number 1.14.19.5.

In the present method, the yeast cell is further capable of expressing a Δ11 fatty acyl desaturase (Δ11 FAD) which can catalyse the conversion of at least part of the hexadecanoyl-CoA into (Z)-11-hexadecenoyl-CoA (FIG. 1). The inventors have found that the Δ11-desaturase from Amyelois transitella (Atr 011; SEQ ID NO: 2) or a variant thereof having at least 70% homology to Atr_Δ11 is well suited for catalyzing this step. In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Atr_Δ11 (SEQ ID NO: 2).

Another suitable Δ11-desaturase is the Spodoptera littoralis Δ11-desaturase (SI_Δ11; SEQ ID NO: 41) or a variant thereof having at least 70% homology to Atr_Δ11. In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SI_Δ11 (SEQ ID NO: 41).

In other embodiments, the Δ11-desaturase is the Agrotis segetum Δ11-desaturase (As_Δ11; SEQ ID NO: 43) or a variant thereof having at least 70% homology to Atr_Δ11. In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to As_Δ11 (SEQ ID NO: 43).

In other embodiments, the Δ11-desaturase is the Trichoplusia ni Δ11-desaturase (Tni_Δ11; SEQ ID NO: 45) or a variant thereof having at least 70% homology to Atr_Δ11. In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Tni_Δ11 (SEQ ID NO: 45).

In some embodiments, the Δ11 FAD can catalyse the conversion of all of the hexadecanoyl-CoA produced in the previous biosynthetic step into (Z)-11-hexadecenoyl-CoA.

Alcohol-Forming Fatty Acyl-CoA Reductase (EC 1.2.1.84)

The terms ‘alcohol-forming fatty acyl-CoA reductase’, ‘fatty acyl-CoA reductase’ and ‘FAR’ will be used herein interchangeably.

The next step in the biosynthesis pathway of (Z)-11-hexadecen-1-ol is the conversion of at least part of the (Z)-11-hexadecenoyl-CoA to (Z)-11-hexadecen-1-ol by an alcohol-forming fatty acyl-CoA reductase (FAR). The FARs capable of catalyzing this conversion can catalyse two consecutive reduction reactions; first, the fatty acyl-CoA is reduced to a fatty aldehyde; second, the fatty aldehyde is further reduced into a fatty alcohol.

The FARs capable of catalyzing such reaction are alcohol-forming fatty acyl-CoA reductases with an EC number 1.2.1.84.

In some embodiments, the FAR is selected from the group consisting of Har_FAR (SEQ ID NO: 8, FAR from Helicoverpa armigera), Hs_FAR (SEQ ID NO: 16, FAR from Heliothis subflexa), and Has_FAR (SEQ ID NO: 12, FAR from Helicoverpa assulta), or a variant thereof having at least 75% homology.

In one embodiment, the FAR is Har_FAR (SEQ ID NO: 8, FAR from Helicoverpa armigera) or a variant thereof having at least 75% homology to Har_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Har_FAR (SEQ ID NO: 8).

In another embodiment, the FAR is Hs_FAR (SEQ ID NO: 16, FAR from Heliothis subflexa), or a variant thereof having at least 75% homology to Hs_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Hs_FAR (SEQ ID NO: 16).

In yet another embodiment, the FAR is Has_FAR (SEQ ID NO: 12, FAR from Helicoverpa assulta), or a variant thereof having at least 75% homology to Has_FAR, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Has_FAR.

In some embodiments, the FAR can catalyse the conversion of all of the (Z)-11-hexadecenoyl-CoA produced in the previous biosynthetic step into (Z)-11-hexadecenol.

The yeast cell provided herein is preferably capable of synthesising hexadecanoyl-CoA and is further capable of expressing a Δ11-desaturase and a FAR as described above.

In some embodiments, expression of the Δ11-desaturase and/or of the FAR can be induced, for example if the genes encoding these enzymes are under the control of inducible promoters, as is known in the art. The yeast cell is incubated under suitable conditions, such as in an appropriate medium and at an appropriate temperature as is known to a person of skill in the art. Suitable media supporting yeast growth are known in the art and include, but are not limited to: undefined, complete media such as YEPD (or YPD, Yeast Extract Peptone Dextrose); defined, complete medium such as SC (Synthetic Complete); defined, drop-out medium such as SD (Synthetic Dextrose) lacking one or more elements such as an amino acid or an inducer; or mineral medium, consisting of salts, vitamins and a carbon source, and others.

Titre

Herein are disclosed methods to produce (Z)-11-hexadecen-1-ol with a titre of at least 0.2 mg/L. In some embodiments, the titre of (Z)-11-hexadecen-1-ol produced by the present methods is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

In one embodiment, the Δ11-desaturase is Atr_Δ11 as set forth in SEQ ID NO: 2 or a variant thereof having at least 70% homology to Atr_Δ11 and the FAR is Har_FAR as set forth in SEQ ID NO: 8 or a variant thereof having at least 75% homology to Har_FAR. In a particular embodiment, the variant of Har_FAR is as set forth in SEQ ID NO: 10.

In one embodiment, the Δ11-desaturase is Atr_Δ11 as set forth in SEQ ID NO: 2 or a variant thereof having at least 70% homology to Atr_Δ11 and the FAR is Hs_FAR as set forth in SEQ ID NO: 16 or a variant thereof having at least 75% homology to Hs_FAR. In a particular embodiment, the variant of Hs_FAR is as set forth in SEQ ID NO: 16.

In one embodiment, the Δ11-desaturase is Atr_Δ11 as set forth in SEQ ID NO: 2 or a variant thereof having at least 70% homology to Atr_Δ11 and the FAR is Has_FAR as set forth in SEQ ID NO: 12 or a variant thereof having at least 75% homology to Has_FAR. In a particular embodiment, the variant of Has_FAR is as set forth in SEQ ID NO: 12.

In another embodiment, the Δ11-desaturase is As_Δ11 as set forth in SEQ ID NO: 43 or a variant thereof having at least 70% homology to As_Δ11 and the FAR is Har_FAR as set forth in SEQ ID NO: 8 or a variant thereof having at least 75% homology to Har_FAR. In a particular embodiment, the variant of Har_FAR is as set forth in SEQ ID NO: 10.

In another embodiment, the Δ11-desaturase is SI_Δ11 as set forth in SEQ ID NO: 41 or a variant thereof having at least 70% homology to SI_Δ11 and the FAR is Har_FAR as set forth in SEQ ID NO: 8 or a variant thereof having at least 75% homology to Har_FAR. In a particular embodiment, the variant of Har_FAR is as set forth in SEQ ID NO: 10.

In another embodiment, the Δ11-desaturase is Tni_Δ11 as set forth in SEQ ID NO: 45 or a variant thereof having at least 70% homology to Tni_Δ11 and the FAR is Har_FAR as set forth in SEQ ID NO: 45 or a variant thereof having at least 75% homology to Har_FAR. In a particular embodiment, the variant of Har_FAR is as set forth in SEQ ID NO: 10.

Production of (Z)-11-hexadecen-1-yl acetate

While the present disclosure provides methods for producing (Z)-11-hexadecenol, it may be of interest to further convert said (Z)-11-hexadecen-1-ol to the corresponding acetate, i.e. (Z)-11-hexadecen-1-yl acetate. Thus in some embodiments, the present method further comprises the step of converting at least part of the (Z)-11-hexadecen-1-ol to (Z)-11-hexadecen-1-yl acetate.

In some embodiments, this is done by further expressing an acetyltransferase (AcT, EC 2.3.1.84) or overexpressing a native acetyltransferase from said yeast cell, wherein said acetyltransferase is capable of converting at least part of the (Z)-11-hexadecen-1-ol into (Z)-11-hexadecen-1-yl acetate, thereby further producing (Z)-11-hexadecen-1-yl acetate.

In the present disclosure, the terms ‘acetyltransferase’, ‘alcohol O-acetyltransferase’ and ‘AcT’ will be used interchangeably.

In other embodiments, the conversion of at least part of the (Z)-11-hexadecen-1-ol to (Z)-11-hexadecen-1-yl acetate is done chemically. The skilled person knows how to convert at least part of the (Z)-11-hexadecen-1-ol to (Z)-11-hexadecen-1-yl acetate. For example, acetyl chloride can be added to the (Z)-11-hexadecen-1-ol and incubated at room temperature after mixing.

In some embodiments, the acetyltransferase is the AcT of SEQ ID NO: 39 (Atf1, the S. cerevisiae AcT) or a variant thereof having at least 75% homology to Sc_Atf1, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 39.

In some embodiments, (Z)-11-hexadecen-1-yl acetate is produced with a titre of at least 0.2 mg/L. In some embodiments, the titre of (Z)-11-hexadecen-1-yl acetate produced by the present methods is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titre are known in the art.

Nucleic Acid Constructs Encoding Δ11-Desaturase, FAR, FAA, AcT

It will be understood that throughout the present disclosure, the term ‘nucleic acid encoding an activity’ shall refer to a nucleic acid molecule capable of encoding a peptide, a protein or a fragment thereof having said activity. Such nucleic acid molecules may be open reading frames or genes or fragments thereof. The nucleic acid construct may also be a group of nucleic acid molecules, which together may encode several peptides, proteins or fragments thereof having an activity of interest. The term ‘activity of interest’ shall refer to one of the following activities: Δ11-desaturase, FAR, FAA and/or AcT activities. The nature of the one or more activity of interest will depend on the nature of the desired product one wishes to obtain with the present methods.

In some embodiments of the present methods, each of the nucleic acids encoding each of the present activities, i.e. Δ11-desaturase, FAR, FAA and/or AcT, may be comprised within the genome of the yeast cell or within a vector comprised within yeast cell.

In some embodiments, each of the nucleic acids encoding each of the present activities may be integrated in the genome of said yeast cell, either because the nucleic acid encodes a native protein, or because it has been integrated therein by genome engineering or genome editing or by crossing yeast cells of different mating types. Methods for integrating a nucleic acid are well known in the art. Thus in some embodiments the activity of interest is encoded by introduction of a heterologous nucleic acid in the yeast cell. The heterologous nucleic acid encoding said activity may be codon-optimised, or may comprise features that can help improve the activity. For example, the heterologous nucleic acid may be modified so as to encode a modified FAA. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognise that such methods include, but are not limited to: cloning and homologous recombination-based methods. Cloning methods may involve the design and construction of a plasmid in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids encoding the activities of interest may be present in high copy number.

In some embodiments, the Δ11-desaturase activity is encoded by a nucleic acid sequence identical to or having at least 80% homology to SEQ ID NO: 1, SEQ ID NO: 40, SEQ ID NO: 42 or SEQ ID NO: 44.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1. In preferred embodiments, the nucleic acid sequence has at least 90% homology to SEQ ID NO: 1.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 40, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 40. In preferred embodiments, the nucleic acid sequence has at least 90% homology to SEQ ID NO: 40.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 42, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 42. In preferred embodiments, the nucleic acid sequence has at least 90% homology to SEQ ID NO: 42.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 44, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 44. In preferred embodiments, the nucleic acid sequence has at least 90% homology to SEQ ID NO: 44.

In some embodiments, the FAR activity is encoded by a nucleic acid sequence identical to or having at least 90% homology to SEQ ID NO: 7, SEQ ID NO: 11 or SEQ ID NO: 15.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7. In preferred embodiments, the nucleic acid sequence has at least 97% homology to SEQ ID NO: 7.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11. In preferred embodiments, the nucleic acid sequence has at least 96% homology to SEQ ID NO: 11.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15. In preferred embodiments, the nucleic acid sequence has at least 97% homology to SEQ ID NO: 15.

In some embodiments, the FAA activity is encoded by a nucleic acid sequence identical to or having at least 65% homology to SEQ ID NO: 34 or at least 65% homology SEQ ID NO: 36.

In some embodiments, the nucleic acid sequence identical to or having at least 65% homology to SEQ ID NO: 34 or SEQ ID NO: 36 has at least 70% homology to SEQ ID NO: 34, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34. In preferred embodiments, the nucleic acid sequence has at least 90% homology to SEQ ID NO: 34.

In some embodiments, the nucleic acid sequence identical to or having at least 65% homology to SEQ ID NO: 34 or 36 has at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36. In preferred embodiments, the nucleic acid sequence has at least 90% homology to SEQ ID NO: 36.

In some embodiments, the FAA activity is encoded by a nucleic acid sequence identical to or having at least 65% homology to SEQ ID NO: 46, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 46.

In some embodiments, the AcT activity is encoded by a nucleic acid sequence identical to or having at least 65% homology to SEQ ID NO: 38.

In some embodiments, the nucleic acid sequence identical to or having at least 65% homology to SEQ ID NO: 38 has at least 70% homology to SEQ ID NO: 38, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 38. In preferred embodiments, the nucleic acid sequence has at least 90% homology to SEQ ID NO: 38.

It follows that herein is disclosed a method for producing (Z)-11-hexadecen-1-ol in a yeast cell as described above, wherein:
the Δ11-desaturase is encoded by a nucleic acid sequence identical to or having at least 80% homology to SEQ ID NO: 1, SEQ ID NO: 40, SEQ ID NO: 42 or SEQ ID NO: 44; and
FAR is encoded by a nucleic acid sequence identical to or having at least 90% homology to SEQ ID NO: 7, SEQ ID NO: 11 or SEQ ID NO: 15, and
FAA is encoded by a native nucleic acid sequence.

Also disclosed herein is a method for producing (Z)-11-hexadecen-1-ol in a yeast cell as described above, wherein:
The Δ11-desaturase is encoded by a nucleic acid sequence identical to or having at least 80% homology to SEQ ID NO: 1, SEQ ID NO: 40, SEQ ID NO: 42 or SEQ ID NO: 44; and
FAR is encoded by a nucleic acid sequence identical to or having at least 90% homology to SEQ ID NO: 7, SEQ ID NO: 11 or SEQ ID NO: 15, and
FAA is encoded by a nucleic acid sequence identical to or having at least 65% homology to SEQ ID NO: 34 or at least 65% homology to SEQ ID NO:

36.

Also disclosed herein is a method for producing (Z)-11-hexadecen-1-yl acetate and/or (Z)-11-hexadecenol, wherein:
The Δ11-desaturase is encoded by a nucleic acid sequence identical to or having at least 80% homology to SEQ ID NO: 1, SEQ ID NO: 40, SEQ ID NO: 42 or SEQ ID NO: 44; and/or
FAR is encoded by a nucleic acid sequence identical to or having at least 90% homology to SEQ ID NO: 7, SEQ ID NO: 11 or SEQ ID NO: 15; and/or
FAA is encoded by a nucleic acid sequence identical to or having at least 65% homology to SEQ ID NO: 34 or at least 65% homology to SEQ ID NO: 36; and/or
AcT is encoded by a nucleic acid sequence identical to or having at least 65% homology to SEQ ID NO: 38.

Also disclosed herein is a method for producing (Z)-11-hexadecen-1-yl acetate and/or (Z)-11-hexadecenol, wherein:
The Δ11-desaturase is encoded by a nucleic acid sequence identical to or having at least 80% homology to SEQ ID NO: 1, SEQ ID NO: 40, SEQ ID NO: 42 or SEQ ID NO: 44; and
FAR is encoded by a nucleic acid sequence identical to or having at least 90% homology to SEQ ID NO: 7, SEQ ID NO: 11 or SEQ ID NO: 15; and
FAA is encoded by a nucleic acid sequence identical to or having at least 65% homology to SEQ ID NO: 34 or at least 65% homology to SEQ ID NO: 36; and
AcT is encoded by a nucleic acid sequence identical to or having at least 65% homology to SEQ ID NO: 38.

Production of (Z)-11-Hexadecenal

While the present disclosure provides methods for producing (Z)-11-hexadecen-1-ol and/or (Z)-11-hexadecen-1-yl acetate, it may be of interest to further convert said (Z)-11-hexadecen-1-ol to the corresponding aldehyde, i.e. (Z)-11-hexadecenal. Thus in some embodiments, the method may further comprise the step of converting at least part of the (Z)-11-hexadecen-1-ol to (Z)-11-hexadecenal, thereby further producing (Z)-11-hexadecenal.

In some embodiments, the step of converting at least part of the (Z)-11-hexadecen-1-ol to (Z)-11-hexadecenal is a step of chemical conversion. The chemical conversion is based on the oxidation of (Z)-11-hexadecen-1-ol to (Z)-11-hexadecenal. Methods for performing this conversion are known in the art. Preferred methods are environmentally friendly and minimize the amount of hazardous waste.

Thus in some embodiments, the chemical conversion may be metal free, avoiding toxic heavy metal based reagents such as manganese oxides, chromium oxides (Jones ox. PDC, PCC) or ruthenium compounds (TPAP, Ley-Griffith ox.). In some embodiments, the conversion does not involve reactions involving activated dimethyl sulfoxide such as the Swern oxidation or the Pfitzner-Moffat type. Such reactions may involve the stereotypic formation of traces of intensively smelling organic sulfur compounds such as dimethyl sulfide which can be difficult to remove from the target product.

In some embodiments, the method comprises a Dess-Martin reaction (Yadav et al., 2004, Meyer et al., 1994).

In other embodiments, the chemical conversion comprises the oxidation with sodium hypochlorite under aqueous/organic two phase conditions (Okada et al., 2014; Tamura et al., 2012; Li et al., 2009).

In some embodiments, the chemical oxidation can be performed with 1-chlorobenzotriazole in a medium of methylene chloride containing 25% pyridine (Ferrell and Yao, 1972).

Alternatively, the oxidation of (Z)-11-hexadecen-1-ol to (Z)-11-hexadecenal can be performed enzymatically by alcohol dehydrogenases. The skilled person will know how to carry out enzymatic oxidation. For example, enzymatic oxidation can be carried out by contacting purified enzymes, cell extracts or whole cells, with (Z)-11-hexadecenol.

Recovery

It may be desirable to recover the products obtained by the methods disclosed herein. Thus the present methods may comprise a further step of recovering (Z)-11-hexadecenol, (Z)-11-hexadecenal and/or (Z)-11-hexadecen-1-yl-acetate.

In some embodiments, the method comprises a step of recovering (Z)-11-hexadecenol. In other embodiments, the method comprises a step of recovering (Z)-11-hexadecen-1-yl-acetate

Methods for recovering the products obtained by the present invention are known in the art and may comprise an extraction with a hydrophobic solvent such as decane, hexane or a vegetable oil.

Production of (Z)-11-hexadecenal

In another aspect, the present disclosure relates to a method for production of (Z)-11-hexadecenal in a yeast cell, said method comprising the steps of:
i) providing a yeast cell capable of synthesising hexadecanoyl-CoA, said yeast cell further capable of expressing:
   the Amyelois transitella Δ11-desaturase (Atr_Δ11; SEQ ID NO: 2), the Spodoptera littoralis Δ11-desaturase (SI_Δ11; SEQ ID NO: 41), the Agrotis segetum Δ11-desaturase (As_Δ11; SEQ ID NO: 43), the Trichoplusia ni Δ11-desaturase (Tni_Δ11; SEQ ID NO: 45) or a variant thereof having at least 70% homology, such as at least 71% homology, such as at least 72%, such as at least 73%, such as at least 74%, such as at least 75%, such as at least 80%, such as at least 85%, such as at least 90%, such as at least 95%, such as 100% homology to Atr_Δ11 (SEQ ID NO: 2), SI_Δ11 (SEQ ID NO: 41), As_Δ11 (SEQ ID NO: 43), or Tni_Δ11 (SEQ ID NO: 45), and
   an aldehyde-forming fatty acyl-CoA reductase (FAR′);
ii) expressing said Δ11-desaturase and said FAR′ from said yeast cell; and
iii) incubating said yeast cell in a medium,
   whereby
The Δ11-desaturase converts at least part of said hexadecanoyl-CoA to (Z)11-hexadecenoyl-CoA; and
said FAR′ converts at least part of said (Z)-11-hexadecenoyl-CoA to (Z)-11-hexadecenal,
   thereby obtaining (Z)-11-hexadecenal.

Yeast Cell

The present disclosure also relates to methods for producing (Z)-11-hexadecenal. In a first step, a yeast cell is provided capable of synthesising hexadecanoyl-CoA.

Any yeast cell capable of synthesising hexadecanoyl-CoA can be used for producing (Z)-11-hexadecenal as described herein.

In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In some embodiments, the genus of said yeast is Saccharomyces or Yarrowia.

The yeast cell may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell or a Yarrowia lipolytica cell.

Fatty acyl-CoA synthetase (FAA)

Hexadecanoyl-CoA is a natural intermediate of fatty acid metabolism, it can further be generated from palmitic acid (either added externally or synthesized inside the cells by thioesterase) via conversion into hexadecanoyl-CoA by FAA. FAA activity is normally present in organisms which are capable of metabolising fatty acids. It may be encoded by several, redundant enzymes. Thus in some embodiments, the yeast cell is further capable of expressing an FAA. The nucleic acid encoding said FAA activity in the yeast cell may be naturally present in the genome of said yeast cell, or it may be introduced by genetic engineering or genome editing. Thus in some embodiments the FAA activity is encoded by introduction of a heterologous nucleic acid in the yeast cell. The heterologous nucleic acid encoding said FAA may be codon-optimised, or may comprise features that can help improve the FAA activity. For example, the heterologous nucleic acid may be modified so as to encode a modified FAA. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the FAA activity can be performed by methods known in the art. The skilled person will recognise that such methods include, but are not limited to: cloning and homologous recombination-based methods. Cloning methods may involve the design and construction of a plasmid in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

In one embodiment, the FAA is Sc_FAA1 (SEQ ID NO: 35) or YI_FAA (SEQ ID NO: 37), or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to Sc_FAA1 (SEQ ID NO: 35) or YI_FAA (SEQ ID NO: 37). Thus in one embodiment, the yeast cell is a S. cerevisiae cell and the FAA is Sc_FAA1 (SEQ ID NO:35) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to Sc_FAA1 (SEQ ID NO: 35). In another embodiment, the yeast cell is a Y. lipolytica cell and the FAA is YI_FAA (SEQ ID NO: 37) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to YI_FAA (SEQ ID NO: 37). In another embodiment, the yeast cell is a S. cerevisiae cell and the FAA is YI_FAA (SEQ ID NO: 37) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to YI_FAA (SEQ ID NO: 37). In yet another embodiment, the yeast cell is a Y. lipolytica cell and the FAA is Sc_FAA1 (SEQ ID NO:35) or a variant thereof having at least 75% homology, such as at least 80% homology, such as at least 85% homology, such as at least 90% homology, such as at least 91% homology, such as at least 92% homology, such as at least 93% homology, such as at least 94% homology, such as at least 95% homology, such as at least 96% homology, such as at least 97% homology, such as at least 98% homology, such as at least 99% homology, such as 100% homology to Sc_FAA1 (SEQ ID NO: 35).

In some embodiments, the FAA is Sc_FAA2 (SEQ ID NO: 47).

Δ11-Fatty Acyl Desaturase (EC 1.14.19.5)

The yeast cell is further capable of expressing a Δ11-fatty acyl desaturase (Δ11 FAD) which can catalyse the conversion of at least part of the hexadecanoyl-CoA into (Z)-11-hexadecenoyl-CoA. The inventors have found that the Δ11-desaturase from Amyelois transitella (Atr_Δ11; SEQ ID NO: 2) or a variant thereof having at least 70% homology to Atr_Δ11 is well suited for catalyzing this step. In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Atr_Δ11 (SEQ ID NO: 2).

In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SI_Δ11 (SEQ ID NO: 41).

In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to As_Δ11 (SEQ ID NO: 43).

In some embodiments, the Δ11 FAD has at least 70% homology, such as at least 71% homology, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to Tni_Δ11 (SEQ ID NO: 45).

In some embodiments, the Δ11 FAD can catalyse the conversion of all of the hexadecanoyl-CoA produced in the previous biosynthetic step into (Z)-11-hexadecenoyl-CoA.

Aldehyde-forming fatty acyl-CoA reductase EC 1.2.1.50 (FAR′)

Instead of producing hexadecen-1-ol, at least part of the (Z)-11-hexadecenoyl-CoA can be converted to (Z)-11 hexadecenal by an aldehyde-forming fatty acyl-CoA reductase (FAR′). The enzymes capable of catalyzing this conversion can catalyse one reduction reaction, where the fatty acyl-CoA is reduced to a fatty aldehyde. Such enzymes are aldehyde-forming fatty acyl-CoA reductases, herein also referred to as FAR′ or aldehyde-forming FAR, with an EC number 1.2.1.50. They catalyse the following reaction: long-chain acyl-CoA+NADPH=long-chain aldehyde+NADP++ coenzyme A, where the term ‘long-chain’ designates chains with 16 to 22 carbon atoms.

The yeast cell provided is thus capable of synthesising hexadecanoyl-CoA and is further capable of expressing a Δ11-desaturase and an aldehyde-forming FAR catalysing a one-step reduction as described above. In some embodiments, expression of the Δ11-desaturase and/or of the aldehyde-forming FAR can be induced, for example if the genes encoding these enzymes are under the control of inducible promoters, as is known in the art. The yeast cell is incubated under suitable conditions, such as in an appropriate medium and at an appropriate temperature as is known to a person of skill in the art. Suitable media supporting yeast growth are known in the art and include, but are not limited to: undefined, complete media such as YEPD (or YPD, Yeast Extract Peptone Dextrose), defined, complete medium such as SC (Synthetic Complete), or defined, drop-out medium such as SD (Synthetic Dextrose) lacking one or more elements such as an amino acid or an inducer.

Titre

Herein are disclosed methods to produce (Z)-11-hexadecenal with a titre of at least 0.1 mg/L. In some embodiments, the titre of (Z)-11-hexadecenal produced by the present methods is at least 0.2 mg/L, such as at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

Nucleic Acids Encoding Δ11-Desaturase, FAR′, FAA, AcT

In some embodiments of the present method for production of (Z)-11-hexadecenal, each of the nucleic acids encoding each of the present activities, i.e. Δ11-desaturase, FAA or aldehyde-forming FAR, may be comprised within the genome of the yeast cell or within a vector comprised within yeast cell.

In some embodiments, each of the nucleic acids encoding each of the present activities may be integrated in the genome of said yeast cell, either because the nucleic acid encodes a native protein, or because it has been integrated therein by genome engineering or genome editing or by crossing yeast cells of different mating types. Methods for integrating a nucleic acid are well known in the art. Thus in some embodiments the activity of interest is encoded by introduction of a heterologous nucleic acid in the yeast cell. The heterologous nucleic acid encoding said activity may be codon-optimised, or may comprise features that can help improve the activity. For example, the heterologous nucleic acid may be modified so as to encode a modified FAA. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognise that such methods include, but are not limited to: cloning and homologous recombination-based methods. Cloning methods may involve the design and construction of a plasmid in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids encoding the activities of interest may be present in high copy number.

In some embodiments, the Δ11-desaturase activity is encoded by a nucleic acid sequence identical to or having at least 80% homology to SEQ ID NO: 1, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1. Preferably, the Δ11-desaturase activity is encoded by a nucleic acid sequence identical to or having at least 90% homology to SEQ ID NO: 1.

In other embodiments, the Δ11-desaturase activity is encoded by a nucleic acid sequence identical to or having at least 80% homology to SEQ ID NO: 40, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 40. Preferably, the Δ11-desaturase activity is encoded by a nucleic acid sequence identical to or having at least 90% homology to SEQ ID NO: 40.

In other embodiments, the Δ11-desaturase activity is encoded by a nucleic acid sequence identical to or having at least 80% homology to SEQ ID NO: 42, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 42. Preferably, the Δ11-desaturase activity is encoded by a nucleic acid sequence identical to or having at least 90% homology to SEQ ID NO: 42.

In other embodiments, the Δ11-desaturase activity is encoded by a nucleic acid sequence identical to or having at least 80% homology to SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 44. Preferably, the Δ11-desaturase activity is encoded by a nucleic acid sequence identical to or having at least 90% homology to SEQ ID NO: 44.

In some embodiments, the FAA activity is encoded by a nucleic acid sequence identical to or having at least 65% homology to SEQ ID NO: 34 or at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34 or SEQ ID NO: 36.

In some embodiments, the FAA activity is encoded by a nucleic acid sequence identical to or having at least 65% homology to SEQ ID NO: 46, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 46.

It follows that herein is disclosed a method for producing (Z)-11-hexadecenal in a yeast cell as described above, wherein:
The Δ11-desaturase is encoded by a nucleic acid sequence identical to or having at least 80% homology to SEQ ID NO: 1, SEQ ID NO: 40, SEQ ID NO: 42 or SEQ ID NO: 44; and
FAR′ is selected from the group consisting of FARs which are able to catalyse a one-step reduction of a fatty acyl-CoA to a fatty aldehyde; and
FAA is encoded by a native nucleic acid sequence.

Also disclosed herein is a method for producing (Z)-11-hexadecen-1-ol in a yeast cell as described above, wherein:
The Δ11-desaturase is encoded by a nucleic acid sequence identical to or having at least 80% homology to SEQ ID NO: 1, SEQ ID NO: 40, SEQ ID NO: 42 or SEQ ID NO: 44; and
FAR′ is selected from the group consisting of FARs which are able to catalyse reduction of a fatty acyl-CoA to a fatty aldehyde, and the further reduction of the fatty aldehyde to a fatty alcohol;
FAA is encoded by a nucleic acid sequence identical to or having at least 65% homology to SEQ ID NO: 34 or SEQ ID NO: 36.

In some embodiments, the FAR′ is an enzyme with an EC number 1.2.1.50.

Recovery

It may be desirable to recover the products obtained by the methods disclosed herein. Thus the present methods may comprise a further step of recovering (Z)-11-hexadecenol, (Z)-11-hexadecenal and/or (Z)-11-hexadecen-1-yl-acetate.

Methods for recovering the products obtained by the present invention are known in the art and may comprise an extraction with a hydrophobic solvent such as decane, hexane or a vegetable oil.

Nucleic Acid Constructs

The present disclosure also relates to a nucleic acid construct comprising one or more of:
a nucleic acid sequence identical to or having at least 80% homology to SEQ ID NO: 1, SEQ ID NO: 40, SEQ ID NO: 42 or SEQ ID NO: 44; and/or
a nucleic acid sequence identical to or having at least 90% homology to SEQ ID NO: 7, SEQ ID NO: 11 or SEQ ID NO: 15; and/or
a nucleic acid sequence identical to or having at least 65% homology to SEQ ID NO: 34 or at least 65% homology to SEQ ID NO: 36; and/or
a nucleic acid sequence identical to or having at least 65% homology to SEQ ID NO: 38.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1. In preferred embodiments, the nucleic acid sequence has at least 90% homology to SEQ ID NO: 1.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 40, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 40. In preferred embodiments, the nucleic acid sequence has at least 90% homology to SEQ ID NO: 40.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 42, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 42. In preferred embodiments, the nucleic acid sequence has at least 90% homology to SEQ ID NO: 42.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 44, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 44. In preferred embodiments, the nucleic acid sequence has at least 90% homology to SEQ ID NO: 44.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7. In preferred embodiments, the nucleic acid sequence identical to or having at least 90% homology to SEQ ID NO: 7 has at least 97% homology to SEQ ID NO: 7.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11. In preferred embodiments, the nucleic acid sequence identical to or having at least 90% homology to SEQ ID NO: 11 has at least 96% homology to SEQ ID NO: 11.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15. In preferred embodiments, the nucleic acid sequence identical to or having at least 90% homology to SEQ ID NO: 15 has at least 97% homology to SEQ ID NO: 15.

In some embodiments, the nucleic acid sequence identical to or having at least 65% homology to SEQ ID NO: 34 or SEQ ID NO: 36 has at least 70% homology to SEQ ID NO: 34, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34. In preferred embodiments, the nucleic acid sequence has at least 90% homology to SEQ ID NO: 34.

In some embodiments, the nucleic acid sequence identical to or having at least 65% homology to SEQ ID NO: 34 or SEQ ID NO: 36 has at least 70% homology to SEQ ID NO: 36, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36. In preferred embodiments, the nucleic acid sequence has at least 90% homology to SEQ ID NO: 36.

In some embodiments, the nucleic acid sequence identical to or having at least 65% homology to SEQ ID NO: 38 has at least 70% homology to SEQ ID NO: 38, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 38. In preferred embodiments, the nucleic acid sequence has at least 90% homology to SEQ ID NO: 38.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44; and
ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44; and
a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44; and
ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44; and
a nucleic acid sequence identical to or having at least 90% homology to SEQ ID NO: 7, SEQ ID NO: 11 or SEQ ID NO: 15; and
a nucleic acid sequence identical to or having at least 65% homology to SEQ ID NO: 34 or at least 65% homology to SEQ ID NO: 36.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7; and
a nucleic acid sequence identical to or having at least 65% homology to SEQ ID NO: 34, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7; and
a nucleic acid sequence identical to or having at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11; and
a nucleic acid sequence identical to or having at least 65% homology to SEQ ID NO: 34, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11; and
a nucleic acid sequence identical to or having at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15; and
a nucleic acid sequence identical to or having at least 65% homology to SEQ ID NO: 34, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15; and
a nucleic acid sequence identical to or having at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36.

In some embodiments, the nucleic acid construct further comprises a nucleic acid sequence identical to or having at least 65% homology to SEQ ID NO: 38, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 38.

At least one of the one or more nucleic acid sequences may be under the control of an inducible promoter.

In some embodiments, the nucleic acid construct is a vector such as an integrative vector or a replicative vector. In one embodiment, the vector is a high copy replicative vector.

Each of the nucleic acid sequences comprised within the present nucleic acid constructs may be present in multiple copies. In some embodiments, at least one of the nucleic acid sequences is present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies. In some embodiments, all of the nucleic acid sequences are present in at least 2 copies, such as at least 3 copies, such as at least 4 copies, such as at least 5 copies, such as at least 10 copies, such as at least 20 copies, such as at least 30 copies, such as at least 40 copies, such as at least 50 copies, such as at least 60 copies, such as at least 70 copies, such as at least 80 copies, such as at least 90 copies, such as at least 100 copies, such as at least 125 copies, such as at least 150 copies, such as at least 175 copies, such as at least 200 copies.

The nucleic acid constructs may also be a PCR product or a synthetic DNA molecule.

Yeast Cell

Also provided herein is a yeast cell that can be used for any of the methods disclosed herein. The yeast cell comprises one or more of:
a nucleic acid sequence identical to or having at least 80% homology to SEQ ID NO: 1, SEQ ID NO: 40, SEQ ID NO: 42 or SEQ ID NO: 44; and/or
a nucleic acid sequence identical to or having at least 90% homology to SEQ ID NO: 7, SEQ ID NO: 11 or SEQ ID NO: 15; and/or
a nucleic acid sequence identical to or having at least 65% homology to SEQ ID NO: 34 or at least 65% homology to SEQ ID NO: 36; and/or
a nucleic acid sequence identical to or having at least 65% homology to SEQ ID NO: 38.

In some embodiments, the yeast cell comprises:
a nucleic acid sequence identical to or having at least 80% homology to SEQ ID NO: 1, SEQ ID NO: 40, SEQ ID NO: 42 or SEQ ID NO: 44; and
a nucleic acid sequence identical to or having at least 90% homology to SEQ ID NO: 7, SEQ ID NO: 11 or SEQ ID NO: 15.

In some embodiments, the yeast cell comprises:
a nucleic acid sequence identical to or having at least 80% homology to SEQ ID NO: 1, SEQ ID NO: 40, SEQ ID NO: 42 or SEQ ID NO: 44; and
a nucleic acid sequence identical to or having at least 90% homology to SEQ ID NO: 7, SEQ ID NO: 11 or SEQ ID NO: 15; and
a nucleic acid sequence identical to or having at least 65% homology to SEQ ID NO: 34, or SEQ ID NO: 36.

In some embodiments, the yeast cell comprises:
a nucleic acid sequence identical to or having at least 80% homology to SEQ ID NO: 1, SEQ ID NO: 40, SEQ ID NO: 42 or SEQ ID NO: 44; and
a nucleic acid sequence identical to or having at least 90% homology to SEQ ID NO: 7, SEQ ID NO: 11 or SEQ ID NO: 15; and
a nucleic acid sequence identical to or having at least 65% homology to SEQ ID NO: 34, or SEQ ID NO: 36; and
a nucleic acid sequence identical to or having at least 65% homology to SEQ ID NO: 38.

As detailed above, the one or more nucleic acid sequence may be comprised within the genome of said yeast cell or within a nucleic acid construct comprised within said yeast cell.

In some embodiments, the yeast cell comprises at least one nucleic acid construct as described above.

Preferably, the yeast cell is capable of synthesising hexadecanoyl-CoA and can be used for producing (Z)-11-hexadecen-1-ol and/or (Z)-11-hexadecenal and/or (Z)-11-hexadecen-1-yl acetate as described herein.

In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In some embodiments, the genus of said yeast is Saccharomyces or Yarrowia.

The yeast cell may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell or a Yarrowia lipolytica cell.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1. In preferred embodiments, the nucleic acid sequence has at least 90% homology to SEQ ID NO: 1.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 40, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 40. In preferred embodiments, the nucleic acid sequence has at least 90% homology to SEQ ID NO: 40.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 42, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 42. In preferred embodiments, the nucleic acid sequence has at least 90% homology to SEQ ID NO: 42.

In some embodiments, the nucleic acid sequence identical to or having at least 80% homology to SEQ ID NO: 1, SEQ ID NO: 40, SEQ ID NO: 42 or SEQ ID NO: 44 has at least 81% homology to SEQ ID NO: 44, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 44. In preferred embodiments, the nucleic acid sequence has at least 90% homology to SEQ ID NO: 44.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7. In preferred embodiments, the nucleic acid sequence identical to or having at least 90% homology to SEQ ID NO: 7 has at least 97% homology to SEQ ID NO: 7.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11. In preferred embodiments, the nucleic acid sequence identical to or having at least 90% homology to SEQ ID NO: 11 has at least 96% homology to SEQ ID NO: 11.

In some embodiments, the nucleic acid sequence identical to or having at least 90% homology to SEQ ID NO: 7, SEQ ID NO: 11 or SEQ ID NO: 15 is a nucleic acid sequence identical to or having at least 90% homology to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15. In preferred embodiments, the nucleic acid sequence identical to or having at least 90% homology to SEQ ID NO: 15 has at least 97% homology to SEQ ID NO: 15.

In some embodiments, the nucleic acid sequence identical to or having at least 65% homology to SEQ ID NO: 34 or SEQ ID NO: 36 has at least 70% homology to SEQ ID NO: 34, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34. In preferred embodiments, the nucleic acid sequence has at least 90% homology to SEQ ID NO: 34.

In some embodiments, the nucleic acid sequence identical to or having at least 65% homology to SEQ ID NO: 34 or SEQ ID NO: 36 has at least 70% homology to SEQ ID NO: 36, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36. In preferred embodiments, the nucleic acid sequence has at least 90% homology to SEQ ID NO: 36.

In some embodiments, the nucleic acid sequence identical to or having at least 65% homology to SEQ ID NO: 38 has at least 70% homology to SEQ ID NO: 34, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 38. In preferred embodiments, the nucleic acid sequence has at least 90% homology to SEQ ID NO: 38.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44; and
a nucleic acid sequence identical to or having at least 90% homology to SEQ ID NO: 7, SEQ ID NO: 11 or SEQ ID NO: 15; and
a nucleic acid sequence identical to or having at least 65% homology to SEQ ID NO: 34 or SEQ ID NO: 36.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44 has at least 90% homology to SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7; and
a nucleic acid sequence identical to or having at least 65% homology to SEQ ID NO: 34, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44 has at least 90% homology to SEQ ID NO: 1, SEQ ID NO: 40, SEQ ID NO: 42 or SEQ ID NO: 44 such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 7; and
a nucleic acid sequence identical to or having at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44 has at least 90% homology to SEQ ID NO: 1, SEQ ID NO: 40, SEQ ID NO: 42 or SEQ ID NO: 44 such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11; and
a nucleic acid sequence identical to or having at least 65% homology to SEQ ID NO: 34, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44 has at least 90% homology to SEQ ID NO: 1, SEQ ID NO: 40, SEQ ID NO: 42 or SEQ ID NO: 44, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 11; and
a nucleic acid sequence identical to or having at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44 has at least 90% homology to SEQ ID NO: 1, SEQ ID NO: 40, SEQ ID NO: 42 or SEQ ID NO: 44, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15; and
a nucleic acid sequence identical to or having at least 65% homology to SEQ ID NO: 34, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 34.

In some embodiments, the nucleic acid construct comprises:
a nucleic acid sequence identical to or having at least 80% homology to SEQ ID NO: 1, SEQ ID NO: 40, SEQ ID NO: 42 or SEQ ID NO: 44 has at least 90% homology to SEQ ID NO: 1, SEQ ID NO: 40, SEQ ID NO: 42 or SEQ ID NO: 44, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 1, SEQ ID NO: 40, SEQ ID NO: 42 or SEQ ID NO: 44;
a nucleic acid sequence identical to or having at least 90% homology to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 15; and
a nucleic acid sequence identical to or having at least 65% homology to SEQ ID NO: 36, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 36.

In some embodiments, the nucleic acid construct further comprises a nucleic acid sequence identical to or having at least 65% homology to SEQ ID NO: 38, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology to SEQ ID NO: 38.

Pheromone Composition

Also provided herein is a pheromone composition comprising (Z)-11-hexadecenol, (Z)-11-hexadecenal or (Z)-11-hexadecen-1-yl acetate. At least one of the (Z)-11-hexadecenol, (Z)-11-hexadecenal or (Z)-11-hexadecen-1-yl acetate is preferably obtainable by the methods disclosed herein above.

In some embodiments, the pheromone composition comprises (Z)-11-hexadecenol, (Z)-11-hexadecenal and (Z)-11-hexadecen-1-yl acetate, where at least one of the (Z)-11-hexadecenol, (Z)-11-hexadecenal or (Z)-11-hexadecen-1-yl acetate is obtainable by the methods disclosed herein above.

Accordingly, the present methods may further comprise the step of formulating the recovered (Z)-11-hexadecenol, (Z)-11-hexadecenal or (Z)-11-hexadecen-1-yl acetate into a pheromone composition. The present pheromone compositions may be used as integrated pest management products, which can be used in a method of monitoring the presence of pest or in a method of disrupting the mating of pest.

Pheromone compositions as disclosed herein may be used as biopesticides. Such compositions can be sprayed or dispensed on a culture, in a field or in an orchard. They can also, as is known in the art, be soaked e.g. onto a rubber septa, or mixed with other components. This can result in mating disruption, thereby preventing pest reproduction, or it can be used in combination with a trapping device to entrap the pests. Non-limiting examples of pests against which the present pheromone compositions can be used are: cotton bollworm (Helicoverpa armigera), striped stemborer (Chilo suppressalis), diamond back moth (Plutella xylostella), cabbage moth (Mamestra brassicae), large cabbage-heart caterpillar (Crocidolomia binotalis), European corn stalk borer (Sesamia nonagrioides), currant clearwing (Synanthedon tipuliformis) and artichoke plume moth (Platyptilia carduidactylal). Accordingly, use of the present compositions on a culture can lead to increased crop yield, with substantially no environmental impact.

The relative amounts of the (Z)-11-hexadecenol, (Z)-11-hexadecenal and (Z)-11-hexadecen-1-yl acetate in the present pheromone compositions may vary depending on the nature of the crop and/or of the pest to be controlled; geographical variations may also exist. Determining the optimal relative amounts may thus require routine optimisation.

Examples of compositions used as repellents can be found in Kehat & Dunkelblum, 1993, for H. armigera, in Alfaro et al., 2009, for C. suppressalis, in Eizaguirre et al., 2002, for S. nonagrioides; in Wu et al., 2012, for P. xylostella; in Bari et al., 2003, for P. carduidactyla

The pheromone composition may thus comprise between 1 and 100% (Z)-11-hexadecenol, between 1 and 100% (Z)-11-hexadecenal and between 1 and 100% (Z)-11-hexadecen-1-yl acetate.

In some embodiments, the pheromone composition may further comprise one or more additional compounds such as a liquid or solid carrier or substrate. For example, suitable carriers or substrate include vegetable oils, refined mineral oils or fractions thereof, rubbers, plastics, silica, diatomaceous earth, wax matrix and cellulose powder.

The pheromone composition may be formulated as is known in the art. For example, it may be under the form of a solution, a gel, a powder. The pheromone composition may be formulated so that it can be easily dispensed, as is known in the art.

Kit of Parts

Also provided herein is a kit of parts comprising a yeast cell, and/or a nucleic acid construct as described herein, and instructions for use.

In some embodiments, the kit comprises a yeast cell that can be used in the methods described herein. In other embodiments, the kit comprises a nucleic acid construct that can be used to engineer a yeast cell useful for the methods described herein. In some embodiments, the kit comprises a yeast cell and a nucleic acid construct as described herein.

### EXAMPLES

### Example 1: Construction of Plasmids and Strains

Four genes encoding Δ11-desaturases from A. transitella (SEQ ID NO: 1), A. segetum (SEQ ID NO: 42), S. littoralis (SEQ ID NO: 40), and T. ni (SEQ ID NO: 44) were synthesized by GeneArt (Life Technologies) in codon-optimized versions for S. cerevisiae. Four genes encoding fatty acyl reductases from A. segetum (SEQ ID NO: 3), H. armigera (SEQ ID NO: 7), H. assulta (SEQ ID NO: 11), and H. subflexa (SEQ ID NO: 15) were also synthesized by GeneArt (Life Technologies) in codon-optimized versions for S. cerevisiae. Additionally several fatty acyl reductases were expressed with altered ER retention signals at their C-termini. Fatty acyl reductase from H. armigera was modified so that its putative native KKSYE signal was replaced with HDEL signal from S. cerevisiae (SEQ ID NO: 9). Fatty acyl reductase from H. subflexa was modified so that its putative native EKKT signal was replaced with HDEL (from S. cerevisiae) (SEQ ID NO: 17). Fatty acyl reductase from H. assulta was modified so that its putative native KKTTNK signal was replaced with HDEL (from S. cerevisiae) (SEQ ID NO: 13). The gene encoding S. cerevisiae alcohol acetyltransferase (AcT) ATF1 was amplified from genomic DNA preparation of S. cerevisiae strain CEN.PK 102-5B.

DNA fragments were amplified by PCR using primers with EasyClone-compatible overhangs as described in (Jensen et al, 2014). The primers are listed in Table 1 and the DNA fragments are listed in Table 2. The PCR mix contained 32 μl water, 10 μl high fidelity Phusion® polymerase buffer (5×), 1 μl dNTPs (10 mM), 1 μl Pfu7× polymerase, 2.5 μl forward primer (10 μM), 2.5 μl reverse primer (10 μM) and 1 μl DNA template and the following PCR program was used: 94° C. for 2 min, 30 cycles of [94° C. for 15 sec, 63° C. for sec, 68° C. for 1 min 30 sec], 68° C. for 2 min, pause at 10° C. The PCR products were separated on a 1%-agarose gel containing Safe-Red® (iNtRON Biotechnology). PCR products of the correct size were excised of the gel and purified using the Nucleospin® Gel and PCR Clean-up kit (Macherey-Nagel).

TABLE 1
Primers.

| | | SEQ ID |
|---|---|---|
| Primer name | Primer sequence, 5′→3′ | NO: |
| PR-8330 (Ase_FAR_U1_fw) | agtgcagguaaaacaatgccagtcttgacttctagag | 19 |
| PR-8331 (Ase_FAR_U1_rev) | cgtgcgauttacttcttcttttcta | 20 |
| PR-8332 (Har_FAR_U1_fw) | agtgcagguaaaacaatggttgtcttgacctccaaag | 21 |
| PR-8336 (Hs_FAR_U1_fw) | agtgcagguaaaacaatggttgtcttgacctc | 22 |
| PR-8337 (Hs_FAR_U1_rev) | cgtgcgauttaagtctttttttcca | 23 |
| PR-8340 (Has_FAR_U1_fw) | agtgcagguaaaacaatggttgtcttgacctc | 24 |
| PR-8341 (Has_FAR_U1_rev) | cgtgcgauttacttgttggtagtct | 25 |
| PR-8350 (Atrd11_U1_fw) | agtgcagguaaaacaatggttccaaacaagggttcc | 26 |
| PR-8351 (Atrd11_U1_rev) | cgtgcgautcatctctttctacccc | 27 |
| PR-10350 (atf1_U1_fw) | agtgcagguaaaacaatgaatgaaatcgatgag | 28 |
| PR-10351 (atf1_U1_rev) | cgtgcgauctaagggcctaaaaggagagctttg | 29 |
| PR-10738 | cgtgcgaUttattcgtagctttttttttccaagaaatgtcta | 30 |
| (Har_FAR_KKSYE_U1_rev) | acac | |
| PR-10739 | cgtgcgaUttacaattcatcatgttccaagaaatgtcta | 31 |
| (Har_FAR_HDEL_U1_rev) | acac | |
| PR-10740 | cgtgcgaUttacaattcatcatgcaagaaatgtctaac | 32 |
| (Hs_FAR_HDEL_U1_rev) | acccc | |
| PR-10741 | cgtgcgaUttacaattcatcatgttccaagaagtgtcta | 33 |
| (Has_FAR_HDEL_U1_rev) | acac | |
| PR-14126 (Ased11_U1_fw) | gtgcaggUaaaacaatggctcaag | 48 |
| PR-14127 (Ased11_U1_rev) | cgtgcgaUttagttgtccttcc | 49 |
| PR-14128 (Sld11_U1_fw) | agtgcaggUaaaacaatggctcaat | 50 |
| PR-14129 (Sld11_U1_rev) | cgtgcgaUtcattcaccctta | 51 |
| PR-14130 (Tnd11_U1_fw) | agtgcaggUaaaacaatggctgttatg | 52 |
| PR-14131 (Tnd11_U1_rev) | cgtgcgaUtcattctttcttagcgtagaaa | 53 |

TABLE 2
DNA fragments obtained by PCR using the indicated template and primers.

| Gene | | | | |
|---|---|---|---|---|
| fragment name | Gene | Fw_primer | Rv_primer | Template DNA |
| BB0684 | Fatty acyl- | PR-8330 | PR-8331 | SEQ ID NO: 3 |
| (Ase_FAR<−) | CoA | (Ase_FAR_U1_fw) | (Ase_FAR_U1_rev) | |
| | reductase | | | |
| | from Agrotis | | | |
| | segetum | | | |
| BB0687 | Fatty acyl- | PR-8336 | PR-8337 | SEQ ID NO: 15 |
| (Hs_FAR<−) | CoA | (Hs_FAR_U1_fw) | (Hs_FAR_U1_rev) | |
| | reductase | | | |
| | from | | | |
| | Heliothis | | | |
| | subflexa | | | |
| BB0689 | Fatty acyl- | PR-8340 | PR-8341 | SEQ ID NO: 11 |
| (Has_FAR<−) | CoA | (Has_FAR_U1_fw) | (Has_FAR_U1_rev) | |
| | reductase | | | |
| | from | | | |
| | Helicoverpa | | | |
| | assulta | | | |
| BB0694 | Δ11- | PR-8350 | PR-8351 | SEQ ID NO: 1 |
| (Atrd11<−) | desaturase | (Atrd11_U1_fw) | (Atrd11_U1_rev) | |
| | from | | | |
| | Amyelois | | | |
| | transitella | | | |
| BB1143 | Alcohol | PR-10350 | PR-10351 | Genomic DNA |
| (ScATF1<−) | acetyltransferase | (atf1_U1_fw) | (atf1_U1_rev) | from S. |
| | from S. | | | cerevisiae |
| | cerevisiae | | | CEN.PK102-5B |
| BB0914 | Fatty acyl- | PR-8332 | PR-10738 | SEQ ID NO: 7 |
| (Har_FAR<−) | CoA | (Har_FAR_U1_fw) | (Har_FAR_KKSYE_U1_rev) | |
| | reductase | | | |
| | from | | | |
| | Helicoverpa | | | |
| | armigera | | | |
| BB0915 | Fatty acyl- | PR-8332 | PR-10739 | SEQ ID NO: 9 |
| (Har_FAR_HDEL<−) | CoA | (Har_FAR_U1_fw) | (Har_FAR_HDEL_U1_rev) | |
| | reductase | | | |
| | from | | | |
| | Helicoverpa | | | |
| | armigera | | | |
| | with | | | |
| | modified C- | | | |
| | terminus | | | |
| BB0916 | Fatty acyl- | PR-8336 | PR-10740 | SEQ ID NO: 17 |
| (Hs_FAR_HDEL<−) | CoA | (Hs_FAR_U1_fw) | (Hs_FAR_HDEL_U1_rev) | |
| | reductase | | | |
| | from | | | |
| | Heliothis | | | |
| | subflexa | | | |
| | with | | | |
| | modified C- | | | |
| | terminus | | | |
| BB0917 | Fatty acyl- | PR-8340 | PR-10741 | SEQ ID NO: 13 |
| (Has_FAR_HDEL<−) | CoA | (Has_FAR_U1_fw) | (Has_FAR_HDEL_U1_rev) | |
| | reductase | | | |
| | from | | | |
| | Helicoverpa | | | |
| | assulta with | | | |
| | modified C- | | | |
| | terminus | | | |
| BB0410 | PTDH3 | PR-1852 | PR-1853 | Genomic DNA |
| (PTDH3<−) | promotor | (PTDH3_fw) | (PTDH3_rv) | from S. |
| | from S. | | | cerevisiae |
| | cerevisiae | | | CEN.PK102-5B |
| BB1354 | Δ11- | PR-14126 | PR-14127 | SEQ ID NO: 42 |
| (Ased11<−) | desaturase | (Ased11_U1_fw) | (Ased11_U1_rev) | |
| | from A. | | | |
| | segetum | | | |
| BB1355 | Δ11- | PR-14128 | PR-14129 | SEQ ID NO: 40 |
| (Sld11<−) | desaturase | (Sld11_U1_fw) | (Sld11_U1_rev) | |
| | from | | | |
| | Spodoptera | | | |
| | littoralis | | | |
| BB1356 | Δ11- | PR-14130 | PR-14131 | SEQ ID NO: 44 |
| (Tnd11<−) | desaturase | (Tnd11_U1_fw) | (Tnd11_U1_rev) | |
| | from | | | |
| | Trichoplusiani | | | |

The basic integrative vectors EasyClone 2.0 pCfB2190 (XI-2-loxP-KILEU2syn), pCfB2228 (XII-4-loxP-SpHIS5syn), and pCfB2190 (XI-2-loxP-KILEU2syn) are described in (Stovicek et al, 2015). Vector for multiple integrations EasyCloneMulti pCfB2047 (pTY2-KIURA3-TAG) is described in (Maury et al, 2016). Additionally we constructed integrative vector pCfB2912 (X1-5-loxP-NatMXsyn). Plasmid pCfB2912 was constructed by USER fusion as described in Stovicek et al. 2015 using BB0593 and BB0598, the original vector backbone and the nourseothricin resistance cassette, respectively.

All basic integrative vectors were linearized with FastDigest® AsiSI (Fermentas) for 2 hours at 37° C. and then nicked with Nb.Bsml (New England Biolabs) for 1 hour at 65° C. The resulting vectors containing sticky ends were separated by gel electrophoresis, excised and gel-purified using the Nucleospin® Gel and PCR Clean-up kit (Macherey-Nagel). The DNA fragments were cloned into the so prepared vectors by USER-cloning via the following protocol: 1 μl of linearized plasmid, 1 μl of promoter fragment, 1.5 μl of gene fragment, 1 μl high fidelity Phusion® polymerase buffer (5×), and 0.5 μl USER enzyme (New England Biolabs) were mixed and incubated at 37° C. for 25 min and at 25° C. for 25 min. The reaction was transformed into chemically competent E. coli DHalpha cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli cultures and the correct cloning was confirmed by sequencing. The constructed integrative vectors are listed in Table 3.

TABLE 3
Integrative expression vectors.

| Integrative expression | Selection | Parent | DNA fragments cloned |
|---|---|---|---|
| vector name | marker | vector | into parent vector |
| pCfB2501 (pXI-2-loxP- | KILEU2 | pCfB2190 | BB0410 (PTDH3<−), |
| KILEU2syn-Ase_FAR- | | | BB0684 (Ase_FAR<−) |
| PTDH3<−) | | | |
| pCfB3412 (pXI-2-loxP- | KILEU2 | pCfB2190 | BB0410 (PTDH3<−), |
| KILEU2syn-Har_FAR- | | | BB0914 (Har_FAR<−) |
| PTDH3<−) | | | |
| pCfB2504 (pXI-2-loxP- | KILEU2 | pCfB2190 | BB0410 (PTDH3<−), |
| KILEU2syn-Hs_FAR- | | | BB0687 (Hs_FAR<−) |
| PTDH3<−) | | | |
| pCfB2506 (pXI-2-loxP- | KILEU2 | pCfB2190 | BB0410 (PTDH3<−), |
| KILEU2syn-Has_FAR- | | | BB0689 (Has_FAR<−) |
| PTDH3<−) | | | |
| pCfB2537 (pXII-4-loxP- | SpHIS5syn | pCfB2228 | BB0410 (PTDH3<−), |
| SpHIS5syn-Atrd11- | | | BB0694 (Atrd11<−) |
| PTDH3<−) | | | |
| pCfB3630 (pXI-5-loxP- | NatMXsyn | pCfB2912 | BB0410 (PTDH3<−), |
| NatMXsyn-ScATF1- | | | BB1143 (ScATF1<−) |
| PTDH3<−) | | | |
| pCfB3413 (pXI-2-loxP- | KILEU2 | pCfB2190 | BB0410 (PTDH3<−), |
| KILEU2syn- | | | BB0915 |
| Har_FAR_HDEL-PTDH3<−) | | | (Har_FAR_HDEL<−) |
| pCfB3414 (pXI-2-loxP- | KILEU2 | pCfB2190 | BB0410 (PTDH3<−), |
| KILEU2syn- | | | BB0916 (Hs_FAR_HDEL<−) |
| Hs_FAR_HDEL-PTDH3<−) | | | |
| pCfB3415 (pXI-2-loxP- | KILEU2 | pCfB2190 | BB0410 (PTDH3<−), |
| KILEU2syn- | | | BB0917 |
| Has_FAR_HDEL-PTDH3 <−) | | | (Has_FAR_HDEL<−) |
| pCfB4369 (pXII-4-loxP- | SpHIS5syn | pCfB2228 | BB0410 (PTDH3<−), |
| SpHIS5syn-Ased11- | | | BB1354 (Ased11<−) |
| PTDH3<−) | | | |
| pCfB4370 (pXII-4-loxP- | SpHIS5syn | pCfB2228 | BB0410 (PTDH3<−), |
| SpHIS5syn-Sld11- | | | BB1355 (Sld11<−) |
| PTDH3<−) | | | |
| pCfB4371 (pXII-4-loxP- | SpHIS5syn | pCfB2228 | BB0410 (PTDH3<−), |
| SpHIS5syn-Tnd11- | | | BB1356 (Tnd11<−) |
| PTDH3<−) | | | |
| pCfB3699 | KIURA3 | pCfB2047 | BB0410 (PTDH3<−), |
| | | | BB0914 (Har_FAR<−) |

The integrative expression vectors were linearized with FastDigest® NotI (Fermentas) and transformed into S. cerevisiae using lithium-acetate protocol (Gietz & Schiestl, 2007). Positive transformants were selected on yeast synthetic drop-out plates (Sigma-Aldrich). Correct integration of the expression constructs into the genome of S. cerevisiae was confirmed by colony PCR.

### Example 2: Production of (Z)-11-Hexadecenol in S. cerevisiae Upon Overexpression of all-Desaturase from Amyelois transitella and Four Variants of Fatty Acyl-CoA Reductase

We constructed S. cerevisiae strains that overexpress Δ11-desaturase from A. transitella and variants of fatty acyl-CoA reductases with native or HDEL C-terminus (Table 4). Three individual isolates of each strain were examined for acquired ability to produce (Z)-11-hexadecen-1-ol. Individual colonies were inoculated in 3 ml yeast synthetic drop-out liquid medium lacking histidine and leucine in 24-deep well microtiter plates with air-penetrable lids (EnzyScreen) and incubated overnight at 30° C. with shaking at 250 rpm. The control parental strain CEN.PK102-5B was cultivated in synthetic complete medium. The next day 60 μl of pre-culture was transferred into 540 μl mineral medium in a 96-deep well microtiter plates with air-penetrable lids (EnzyScreen). The mineral medium had the composition as described in (Jensen et al, 2014). For the control strain the medium was supplemented with 240 mg/L leucine, 76 mg/L histidine and 20 mg/L uracil and the medium for the other strains was supplemented with 20 mg/L uracil. The microtiter plates were incubated at 30° C. with shaking at 250 rpm for 48 hours. After taking a sample for OD, to the remaining 495 ul of culture an equal volume of decane was added. The plate was covered with a lid made of Viton from EnzyScreen (to avoid absorption of the organic solvent into the lid) and the plate was shaken for 10 min at 250 rpm. The resulting emulsion was transferred to a 4 mL-glass vial, closed with a lid, vortexed for 20 sec and centrifuged for 35 sec at 4000×g. The bottom (water) phase was removed and 0.1 g Na₂SO₄ was added to the remaining decane phase. The mix was vortexed for 10 sec and the clear organic phase was transferred to a glass vial for GC-MS analysis. GC-MS analyses were performed on a Hewlett Packard 6890 GC coupled to a mass selective detector HP 5973. The GC was equipped with an INNOWax column (30 m×0.25 mm×0.25 μm), and helium was used as carrier gas (average velocity: 33 cm/s). The MS was operated in electron impact mode (70 eV), scanning between m/z 30 and 400, and the injector was configured in splitless mode at 220° C. The oven temperature was set to 80° C. for 1 min, then increased at a rate of 10° C./min to 210° C., followed by a hold at 210° C. for 15 min, and then increased at a rate of 10° C./min to 230° C. followed by a hold at 230° C. for 20 min. Compounds were identified by comparison of retention times and mass spectra with those of reference compounds available in laboratory collection.

Compounds were quantified by the Total Ion Current (TIC) recorded. Data were analysed by the Agilent ChemStation software and iWork Numbers. The concentration of (Z)-11-hexadecen-1-ol was calculated based on calibration curve ranging from 0 mg/L to 15 mg/L of (Z)-11-hexadecen-1-ol standard. The standards was purchased from Pherobank (Wageningen, Netherlands).

Introduction of Δ11-desaturase from A. transitella and of a fatty acyl-CoA reductase from H. armigera or H. subflexa or H. assulta enabled S. cerevisiae to synthesize (Z)-11-hexadecen-1-ol at 1-3.9 mg/L titer (Table 4). Previous studies have previously reported that production of (Z)-11-hexadecen-1-ol was obtained when expressing Ase011 and Ase_FAR in S. cerevisiae (Hagstrom et al., 2013). However, our results show that Ase_FAR has a very low activity and is much less suitable for production of (Z)-11-hexadecen-1-ol than Har_FAR, Hs_FAR or Has_FAR.

TABLE 4
S. cerevisiae strains engineered for production of (Z)-11-hexadecen-1-
ol with different variants of fatty acyl-CoA reductase.

| Strain | Overexpressed | Parent strain and | (Z)-11-hexadecen- |
|---|---|---|---|
| name | heterologous genes | (integrated vectors) | 1-ol titer [mg/L] |
| ST3328 | AtrΔ11, Ase_FAR | CEN.PK102-5B | 0 ± 0 |
| | | (pCfB2537, pCfB2501) | |
| ST3705 | AtrΔ11, Har_FAR | CEN.PK102-5B | 1.57 ± 0.27 |
| | | (pCfB2537, pCfB3412) | |
| ST3330 | AtrΔ11, Hs_FAR | CEN.PK102-5B | 1.26 ± 0.32 |
| | | (pCfB2537, pCfB2504) | |
| ST3339 | AtrΔ11, Has_FAR | CEN.PK102-5B | 3.28 ± 1.23 |
| | | (pCfB2537, pCfB2506) | |
| ST3706 | AtrΔ11, Har_FAR_HDEL | CEN.PK102-5B | 3.85 ± 1.63 |
| | (SEQ ID NO: 10) | (pCfB2537, pCfB3413) | |
| ST3707 | AtrΔ11, Hs_FAR_HDEL | CEN.PK102-5B | 2.49 ± 0.96 |
| | (SEQ I DNO: 18) | (pCfB2537, pCfB3414) | |
| ST3708 | AtrΔ11, Has_FAR_HDEL | CEN.PK102-5B | 1.02 ± 0.79 |
| | (SEQ ID NO: 14) | (pCfB2537, pCfB3415) | |
| ST10 | None | CEN.PK102-5B (none) | 0 ± 0 |

### Example 3: Production of (Z)-11-Hexadecenol in S. cerevisiae Upon Overexpression of Fatty Acyl-CoA Reductase from H. armigera and Four Variants of Δ11-Desaturase

We constructed S. cerevisiae strains that overexpress fatty acyl-CoA reductase from H. armigera with modified C-terminus and four variants of Δ11-desaturase in order to identify the one with the highest activity (Table 5). Two individual isolates of each strain were examined for acquired ability to produce (Z)-11-hexadecen-1-ol.

Individual colonies were inoculated in 5 ml of Yeast extract-Peptone-Dextrose (YPD) medium with 8% glucose (10 g/L yeast extract, 20 g/L peptone, 80 g/L dextrose) in 12-m1 glass tubes (Duran, Wertheim, Germany) with metal labocap lids (Ladiswiss, Flawil, Switzerland) and incubated overnight at 30° C. with shaking at 250 rpm. The following day the overnight culture was centrifuged, the supernatant was discarded and the pellet was resuspended in 2 mL feed-in-time (FIT) medium supplemented with 20 mg/L uracil. The feed-in-time medium was purchased from m2p-labs GmbH (Baesweiler, Germany). It was supplemented with 0.5% enzyme solution and 1% vitamin solution immediately prior to use. The tubes were incubated at 30° C. with shaking at 250 rpm for 40 hours. For extraction, 1 mL of culture was transferred into a 4-mL glass vial and 10 μL of internal standard stock (1 μg/pL (Z)-10-heptan-1-yl methyl ester in 100% ethanol) was added. The vials were covered with small pieces of aluminum foil and we used a needle to pierce small holes in the foil covers. The samples were vortexed and placed at −80° C. for storage until analysis. The samples were freeze-dried in a freeze dry system (Freezone6 and Stoppening tray dryer, Labconco, Kansas City, USA) at −40° C., then 1 mL chloroform:methanol 2:1 was added to disrupt the cells. The mix was vortexed for 45 s and left at room temperature for 4 hours.

The organic solvents were evaporated slowly under a nitrogen stream. 1 ml of hexane was added, the samples were vortexed for 10 s, centrifuged and 200 μl were transferred to a new glass vial. GC-MS analysis was performed as described in Example 2. The concentration of (Z)-11-hexadecen-1-ol was calculated based on internal standard and calibration curve.

The product (Z)-11-hexadecen-1-ol was produced by all the four tested strains, but Δ11-desaturase from A. transitella was clearly superior to others, resulting in a titer of ca. 7 mg/L. The titer of strain ST3706 was higher than in example 2 due to improved cultivation and extraction protocol.

TABLE 5
S. cerevisiae strains engineered for production of (Z)-11-
hexadecen-1-ol with different variants of Δ11-desaturase.

| CfB | Overexpressed | | (Z)-11- |
|---|---|---|---|
| strain | heterologous | Parent strain and | hexadecen-1- |
| name | genes | (integrated vectors) | ol titer [mg/L] |
| ST3706 | AtrΔ11, | CEN.PK102-5B | 7.1 ± 0.53 |
| | Har_FAR_HDEL | (pCfB2537, pCfB3413) | |
| ST4487 | AseΔ11, | CEN.PK102-5B | 1.8 ± 0.26 |
| | Har_FAR_HDEL | (pCfB4369, pCfB3413) | |
| ST4488 | SlΔ11, | CEN.PK102-5B | 3.2 ± 0.35 |
| | Har_FAR_HDEL | (pCfB4370, pCfB3413) | |
| ST4489 | TniΔ11, | CEN.PK102-5B | 3.5 ± 0.42 |
| | Har_FAR_HDEL | (pCfB4371, pCfB3413) | |

### Example 4: Improved Production of (Z)-11-Hexadecen-1-Ol by Integration of Multiple Copies of Fatty Acyl-CoA Reductase

We constructed S. cerevisiae strains that overexpress Δ11-desaturase from A. transitella and fatty acyl-CoA reductase from H. armigera Har_FAR. The Har_FAR gene was integrated in either a single (ST3705) or multiple copies into the genome (ST5262). ST3705 was inoculated into 5 ml of YPD medium with 8% glucose (10 g/L yeast extract, 20 g/L peptone, 80 g/L dextrose) and ST5262 was inoculated into 5 ml synthetic complete medium lacking uracil. Both strains were cultivated in 12-m1 glass tubes (Duran, Wertheim, Germany) with metal labocap lids (Lüdiswiss, Flawil, Switzerland) and incubated overnight at 30° C. with shaking at 250 rpm. The following day the overnight culture was centrifuged, the supernatant was discarded and the pellet was resuspended in 2 mL feed-in-time (FIT) medium. For strain ST3705 the medium was supplemented with 20 mg/L uracil. The feed-in-time medium was purchased from m2p-labs GmbH (Baesweiler, Germany). It was supplemented with 0.5% enzyme solution and 1% vitamin solution immediately prior to use. The tubes were incubated at 30° C. with shaking at 250 rpm for 40 hours. The sample extraction was performed as in described in Example 3. Integration of multiple Har_FAR gene copies increased production eight fold (Table 6).

TABLE 6
S. cerevisiae strains engineered for production of (Z)-11-hexadecen-1-
ol with different gene copy numbers of fatty acyl reductase.

| CfB | Overexpressed | | (Z)-11- |
|---|---|---|---|
| strain | heterologous | Parent strain and | hexadecen-1- |
| name | genes | (integrated vectors) | ol titer [mg/L] |
| ST3705 | AtrΔ11, Har_FAR | CEN.PK102-5B | 11.0 ± 1.4 |
| | | (pCfB2537, pCfB3412) | |
| ST5262 | AtrΔ11, Har_FAR | CEN.PK102-5B | 85.4 ± 0.8 |
| | (multiple copies | (pCfB2537, pCfB3412, | |
| | integrated) | pCfB3699) | |

### Example 5: Production of (Z)-11-Hexadecen-1-Ol in Fed-Batch Fermentation

Strain ST3705 was tested for production of (Z)-11-hexadecen-1-ol in fed-batch fermentation. About 1 ml of cryo culture of strain ST3705 was inoculated into 150 ml of synthetic complete medium without leucine and histidine (SC leu-his-) in 500-ml-baffled shake flask and incubated shaking at 250 rpm for 24 hours at 30° C. The inoculum was up-concentrated by centrifuging the culture, removing 100 ml of supernatant and resuspending the cells in the remaining 50 ml of liquid. This cell suspension was used to inoculate 500 mL fermentation medium (6 g/L KH2PO4, 4 g/L (NH4)2504, 1 g/L MgSO4*7H2O, 1 ml/L antifoam 204, 4 ml/L trace metals (Jensen, 2013), 2 ml/L vitamins (Jensen, 2013), and 32 mg/L uracil) in a 1 L Sartorius bioreactor. The reactor operating conditions were 30° C., aeration at 1 L/min, and agitation at 800 rpm. The pH was kept at 5 with 2M KOH. The feed solution was composed of 500 ml 200 g/L glucose. 100 ml of feed solution was added to the reactor to start the fermentation. 24 hours later, constant feed rate of 5 g/h was applied and maintained throughout the fermentation.

Strain ST5262 was tested for production of (Z)-11-hexadecen-1-ol in fed-batch fermentation. Strain ST5262 was inoculated into 100 mL double concentrated synthetic complete medium without uracil (2×SC ura-) in 2 L-baffled flask and incubated shaking at 250 rpm for 24 h at 28° C. Additional 400 mL of 2×SC ura-were added and the incubation continued under the same conditions for additional 48 hours. To prepare a concentrated inoculum for the fermentation, the cells were centrifuged and resuspended in 50 mL of the remaining supernatant. This cell suspension was used to inoculate 500 mL fermentation medium (6 g/L KH₂PO₄, 6 g/L (NH₄)₂ SO₄, 1 g/L MgSO₄*7H₂O, 1 ml/L antifoam 204, 4 ml/L trace metals (Jensen, 2013), 2 ml/L vitamins (Jensen, 2013)) in a 1 L Sartorius bioreactor. The reactor operating conditions were 30° C., aeration at 1.5 L/min, and agitation at 800 rpm. Dissolved oxygen was controlled at above 20% by automatic blending of oxygen into the air. The pH was kept at 5 with 10% KOH. The feed solution was composed of 500 ml 330 g/L dextrose supplemented with 0.5 ml antifoam 204. Ca. 20 ml of feed solution was added to the reactor to start the fermentation. The feed rate was set to 5 g/h. The feed rate was increased to 7.5 g/h at 17 hours from the start of fermentation and further to 10 g/h at 34 hours from the start of fermentation. The feed rate was decreased to 3 g/h at 60 hours until the culture was harvested at 100 hours, by which time all the feed was consumed.

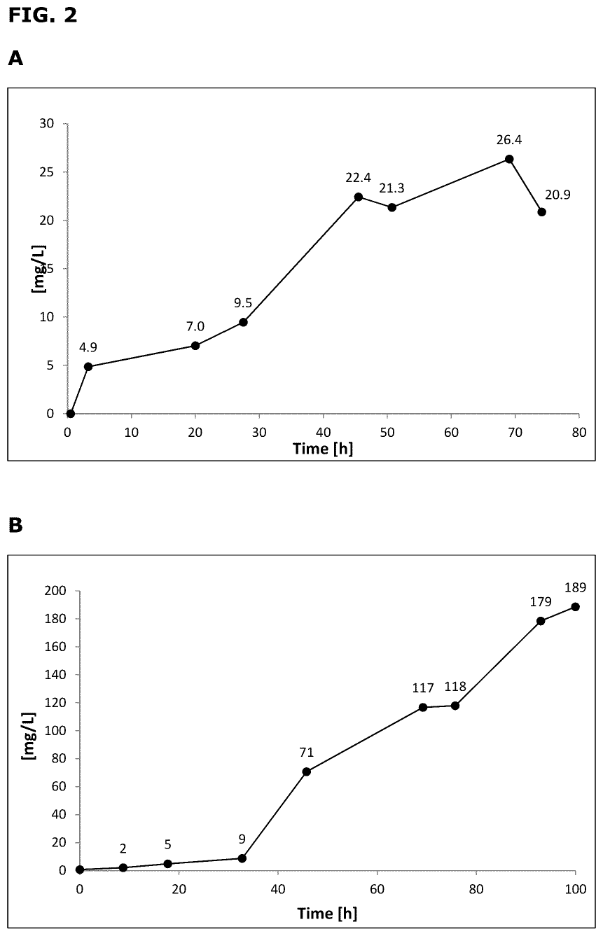

Sampling was performed by withdrawing a few mL broth with a syringe from an outlet in the reactor, transferring the sample to a plastic tube, kept at −20° C. until (Z)-11-hexadecen-1-ol extraction. Extraction and GC-MS analysis were performed as described in Example 3. The final production titre for strain ST3705 was 20 mg/L (FIG. 2A) and for strain ST5262 it was 189 mg/L (FIG. 2B).

### Example 6: Production of (Z)-11-Hexadecen-1-Ol in Yarrowia lipolytica

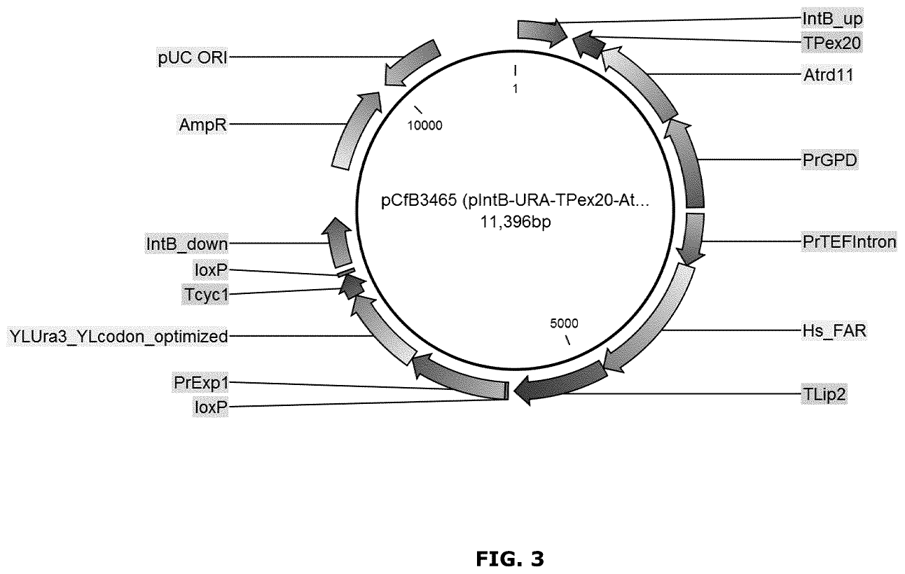

We tested production of (Z)-11-hexadecen-1-ol in Y. lipolytica expressing Δ11-desaturase from A. transitella and fatty acyl-CoA reductase from H. subflexa. Genes encoding an A. transitella Δ11-desaturase (SEQ ID NO: 2) and a H. subflexa fatty acyl-CoA reductase (SEQ ID NO: 16) were cloned into a Y. lipolytica expression vector resulting in plasmid pCfB3465 (FIG. 3). Prior to transformation into Y. lipolytica the expression plasmid was linearized with NotI. The linearized plasmid was transformed into strain Y. lipolytica GB20 (Angerer, 2014) using a lithium-acetate-based protocol (Chen, 1997). Positive transformants were selected on synthetic complete (SC) medium lacking uracil. The integration of the expression construct into the Y. lipolytica genome was confirmed by colony PCR.

One individual clone of each strain was inoculated into 5 ml YPD medium with 8% glucose (10 g/L yeast extract, 20 g/L peptone, 80 g/L dextrose) in 12-m1 glass tubes (Duran, Wertheim, Germany) with metal labocap lids (Ludiswiss, Flawil, Switzerland) and incubated overnight at 30° C. with shaking at 250 rpm. The following day the overnight culture was centrifuged, the supernatant was discarded and the pellet was resuspended in 2 ml nitrogen-limited medium (2.9 g/L (NH₄)₂SO₄, 1.7 g/L YNB (without amino acids and ammonium sulfate, 240 mg/L leucine, 76 mg/L lysine) and 61 g/L glycerol). For strain ST3683 the medium was supplemented with 20 mg/L uracil. The cultures were incubated for 48 hours at 30° C. and shaken at 250 rpm. Extraction and GC-MS analysis were performed as described in Example 3. The Y. lipolytica strain ST3844 expressing A. transitella Δ11-desaturase and H. subflexa fatty acyl-CoA reductase produced 1.7 mg/L of (Z)-11-hexadecen-1-ol (Table 7).

TABLE 7
Strains and (Z)-11-hexadecen-1-ol titres

| CfB strain | Parent | Integrated | (Z)-11-hexadecen- |
|---|---|---|---|
| name | strain | vector | 1-ol titres [mg/L] |
| ST3683 | — | — | 0.20 ± 0.03 |
| ST3844 | ST3683 | pCfB3465 | 1.71 ± 0.14 |

### Example 7: Production of (Z)-11-hexadecen-1-yl Acetate

We constructed strain ST3581, expressing Δ11-desaturase from A. transitella, fatty acyl-CoA reductase from H. subflexa and a S. cerevisiae alcohol acetyltransferase AcT for production of (Z)-11-hexadecen-1-yl acetate.

Individual colonies of ST3581 and the control strain, ST3330, which does not overexpress the alcohol acetyltransferase, were inoculated in 5 ml of YPD medium with 8% glucose (10 g/L yeast extract, 20 g/L peptone, 80 g/L dextrose) in 12-m1 glass tubes (Duran, Wertheim, Germany) with metal labocap lids (Ludiswiss, Flawil, Switzerland) and incubated overnight at 30° C. with shaking at 250 rpm. The following day the overnight culture was centrifuged, the supernatant was discarded and the pellet was resuspended in 2 ml nitrogen-limited mineral medium (1.25 g/L (NH₄)₂SO₄, 14.4 g/L KH₂PO₄, 0.5 g/L MgSO₄*7H₂O, 2 mL/L trace metal solution (Jensen, 2013), 1 mL/L vitamin solution (Jensen, 2013)) with 20 g/L glucose. The cultures were incubated for 48 hours at 30° C. and shaken at 250 rpm. Extraction and GC-MS analysis was performed as described in Example 3. ST3581 produced 1.68 mg/L (Z)-11-hexadecen-1-yl acetate, while no (Z)-11-hexadecen-1-yl acetate production could be observed in the control strain ST3330 (Table 8).

TABLE 8
Strains and (Z)-11-hexadecen-1-yl acetate titers.

| CfB | | | | (Z)-11- |
|---|---|---|---|---|
| strain | Parent | Overexpressed | Integrated | hexadecen-1-yl |
| name | Strain | genes | plasmids | acetate (mg/L) |
| ST3581 | CEN.PK102- | AtrΔ11, | pCfB2537, | 1.68 ± 0.17 |
| | 5B | Hs_FAR, | pCfB2504, | |
| | | ScATF1 | pCfB3630 | |
| ST3330 | CEN.PK102- | AtrΔ11, | pCfB2537, | 0.00 ± 0.00 |
| | 5B | Hs_FAR | pCfB2504 | |

Sequences

SEQ ID NO: 1-S. cerevisiae-codon-optimized nucleotide sequence of A. transitella Δ11-
desaturase; mRNA-coding sequence.
Atggttccaaacaagggttcctctgatgttttgtctgaacattctgaaccacaattcaccaagttgattgctccacaagctggtccaa
gaaagtacaaaatcgtttacagaaacttgttgaccttcggttactggcatttgtctgctgtttatggtttgtacttgtgtttcacttgtgcta
agtgggctactattttgttcgctttcttcttgtacgttatcgccgaaattggtattactggtggtgctcatagattatgggctcatagaactt
acaaagccaagttgccattggaaatcttgttgttgatcatgaactccattgccttccaagatactgcttttacttgggctagagatcat
agattgcatcacaagtactctgatactgatgctgatccacataatgctactagaggtttcttctactctcatgttggttggttgttggtta
agaaacacccagaagttaaggctagaggtaagtacttgtctttggatgacttgaagaacaaccctttgttgaagttccaaaaga
agtacgccattttggtcattggtactttgtgctttttgatgccaactttcgttccagtttacttttggggtgaaggtatttctactgcctggaa
cattaacttgttaagatacgtcatgaacttgaacatgacctttttggttaactccgctgctcatatttttggtaacaagccatacgataa
gtctatcgcctctgttcaaaacatctctgtttctttggctactttcggtgaaggtttccataactaccatcatacttatccatgggattaca
gagctgctgaattgggtaacaatagattgaatatgaccaccgccttcattgatttctttgcttggattggttgggcctacgatttgaaa
tctgttccacaagaagctattgctaagagatgtgctaaaactggtgatggtactgatatgtggggtagaaagagatga
SEQ ID NO: 2-Amino acid sequence of A. transitella delta-11-desaturase (translation)
MVPNKGSSDVLSEHSEPQFTKLIAPQAGPRKYKIVYRNLLTFGYWHLSAVYGLYLCFTCAK
WATILFAFFLYVIAEIGITGGAHRLWAHRTYKAKLPLEILLLIMNSIAFQDTAFTWARDHRLHH
KYSDTDADPHNATRGFFYSHVGWLLVKKHPEVKARGKYLSLDDLKNNPLLKFQKKYAILVI
GTLCFLMPTFVPVYFWGEGISTAWNINLLRYVMNLNMTFLVNSAAHIFGNKPYDKSIASVQ
NISVSLATFGEGFHNYHHTYPWDYRAAELGNNRLNMTTAFIDFFAWIGWAYDLKSVPQEAI
AKRCAKTGDGTDMWGRKR
SEQ ID NO: 3-S. cerevisiae-codon-optimized nucleotide sequence of A. segetum fatty
acyl reductase; mRNA-coding sequence.
atgccagtcttgacttctagagaagacgaaaaattgtccgtcccagaattttacgctggtaagtctatttttgttaccggtggtactgg
tttcttgggtaaggtttttatcgaaaagttgttgtactgctgcccagatatcgataagatctacatgttgatcagagaaaaaaagaac
ttgtccatcgacgaaagaatgtccaagtttttggatgaccctttgttctccagattgaaagaagaaagaccaggtgacttggaaa
agatcgttttgattccaggtgatattaccgctcctaatttgggtttgtctgctgaaaacgaaagaatcttgttggaaaaggtcagtgtc
attattaactctgctgctaccgttaagttcaacgaaccattgccaattgcttggaagattaacgttgaaggtactagaatgttgttggc
cttgtctagaagaatgaagagaatcgaagttttcatccatatctccaccgcttactctaatgcttcttctgatagaattgtcgttgacg
aaatcttgtatccagctccagctgatatggatcaagtttatcaattggttaaggacggtgtcactgaagaagaaaccgaaagatt
attgaacggtttgccaaacacttacactttcactaaggctttgaccgaacatttggttgctgaacatcaaacttacgttccaaccatt
atcatcagaccatctgttgttgcctccattaaggatgaacctattagaggttggttgtgtaattggtttggtgctactggtatttctgttttc
actgctaagggtttgaacagagttttgttgggtaaagcctctaacatcgttgatgttatcccagttgattacgttgccaacttggttata
gttgctggtgctaaatctggtggtcaaaagtctgatgaattgaaaatctacaactgctgctcctctgactgtaatccagttactttgaa
gaagatcatcaaagaattcaccgaagataccatcaagaacaagtcccatattatgccattgccaggttggttcgtttttactaagt
acaaatggttgttgactttgttgaccatcatcttccaaatgttgccaatgtatttggccgatgtttacagagtcttgaccggtaaaattc
caagatatatgaagttgcaccacttggtcattcaaaccagattgggtattgatttcttcacctctcattcttgggttatgaagaccgat
agagtcagagaattattcggttctttgtccttggccgaaaaacacatgtttccatgtgatccatcttccattgattggaccgattacttg
caatcttactgctatggtgtcagaagattcttagaaaagaagaagtaa
SEQ ID NO: 4-Amino acid sequence of A. segetum fatty acyl reductase
MPVLTSREDEKLSVPEFYAGKSIFVTGGTGFLGKVFIEKLLYCCPDIDKIYMLIREKKNL
SIDERMSKFLDDPLFSRLKEERPGDLEKIVLIPGDITAPNLGLSAENERILLEKVSVIINSAATV
KFNEPLPIAWKINVEGTRMLLALSRRMKRIEVFIHISTAYSNASSDRIVVDEILYPAPADMDQ
VYQLVKDGVTEEETERLLNGLPNTYTFTKALTEHLVAEHQTYVPTIIIRPSVVASIKDEPIRG
WLCNWFGATGISVFTAKGLNRVLLGKASNIVDVIPVDYVANLVIVAGAKSGGQKSDELKIYN
CCSSDCNPVTLKKIIKEFTEDTIKNKSHIMPLPGWFVFTKYKWLLTLLTIIFQMLPMYLADVY
RVLTGKIPRYMKLHHLVIQTRLGIDFFTSHSWVMKTDRVRELFGSLSLAEKHMFPCDPSSID
WTDYLQSYCYGVRRFLEKKK
SEQ ID NO: 5-S. cerevisiae-codon-optimized nucleotide sequence of H. armigera fatty
acyl reductase with signal peptide changed to KKYR; mRNA-coding sequence.
atggttgtcttgacctccaaagaaactaagccatctgttgctgaattttacgctggtaagtctgttttcattactggtggtactggtttctt
gggtaaggttttcattgaaaagttgttgtactcctgcccagatatcggtaatatctacatgttgatcagagaaaagaagggtttgtcc
gtttccgaaagaatcaagcactttttggatgatcctttgttcaccagattgaaagaaaaaagaccagccgacttggaaaagatcg
ttttgattccaggtgatattactgctccagatttgggtattacctccgaaaacgaaaagatgttgatcgaaaaggtcagtgtcattatt
cattctgctgctaccgttaagttcaacgaaccattgccaactgcttggaagattaacgttgaaggtactagaatgatgttggccttgt
ctagaagaatgaagagaatcgaagttttcatccatatctctaccgcttacactaacaccaacagagaagttgttgacgaaatctt
gtatccagctccagctgatattgatcaagttcacagatatgttaaggacggtatctctgaagaagaaactgaaaaaatcttgaac
ggtagaccaaacacttacactttcactaaggctttgaccgaacatttggttgctgaaaatcaagcttacgttccaaccattatcgtta
gaccatcagttgttgctgccattaaggatgaacctattaagggttggttgggtaattggtatggtgctacaggtttgactgtttttactg
ctaagggtttgaacagagttatctacggtcactcttctaacatcgttgatttgatcccagttgattacgttgccaacttggttattgctgc
tggtgctaaatcttctaagtctactgaattgaaggtctacaactgctgttcttctgcttgtaacccaattactatcggtaagttgatgtcc
atgtttgctgaagatgctatcaagcaaaagtcttacgctatgccattgccaggttggtacatttttactaagtacaagtggttggtctt
gttgttgaccattttgttccaagttattccagcctacattaccgacttgtacagacatttgattggtaagaacccaagatatatcaagtt
gcaatccttggtcaatcaaaccagatcctccattgatttcttcacctctcattcttgggttatgaaggctgatagagtcagagaattatt
cgcttctttgtctccagcagataagtacttgtttccatgtgatccaaccgatattaactggacccattacattcaagattactgctggg
gtgttagacatttcttggaaaaaaagtacagataa
SEQ ID NO: 6-Amino acid sequence of H. armigera fatty acyl reductase with signal
peptide changed to KKYR
MVVLTSKETKPSVAEFYAGKSVFITGGTGFLGKVFIEKLLYSCPDIGNIYMLIREKKGLS
VSERIKHFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
RYVKDGISEEETEKILNGRPNTYTFTKALTEHLVAENQAYVPTIIVRPSVVAAIKDEPIKGWL
GNWYGATGLTVFTAKGLNRVIYGHSSNIVDLIPVDYVANLVIAAGAKSSKSTELKVYNCCSS
ACNPITIGKLMSMFAEDAIKQKSYAMPLPGWYIFTKYKWLVLLLTILFQVIPAYITDLYRHLIG
KNPRYIKLQSLVNQTRSSIDFFTSHSWVMKADRVRELFASLSPADKYLFPCDPTDINWTHYI
QDYCWGVRHFLEKKYR
SEQ ID NO: 7-S. cerevisiae-codon-optimized nucleotide sequence of H. armigera fatty
acyl reductase; mRNA-coding sequence.
atggttgtcttgacctccaaagaaactaagccatctgttgctgaattttacgctggtaagtctgttttcattactggtggtactggtttctt
gggtaaggttttcattgaaaagttgttgtactcctgcccagatatcggtaatatctacatgttgatcagagaaaagaagggtttgtcc
gtttccgaaagaatcaagcactttttggatgatcctttgttcaccagattgaaagaaaaaagaccagccgacttggaaaagatcg
ttttgattccaggtgatattactgctccagatttgggtattacctccgaaaacgaaaagatgttgatcgaaaaggtcagtgtcattatt
cattctgctgctaccgttaagttcaacgaaccattgccaactgcttggaagattaacgttgaaggtactagaatgatgttggccttgt
ctagaagaatgaagagaatcgaagttttcatccatatctctaccgcttacactaacaccaacagagaagttgttgacgaaatctt
gtatccagctccagctgatattgatcaagttcacagatatgttaaggacggtatctctgaagaagaaactgaaaaaatcttgaac
ggtagaccaaacacttacactttcactaaggctttgaccgaacatttggttgctgaaaatcaagcttacgttccaaccattatcgtta
gaccatcagttgttgctgccattaaggatgaacctattaagggttggttgggtaattggtatggtgctacaggtttgactgtttttactg
ctaagggtttgaacagagttatctacggtcactcttctaacatcgttgatttgatcccagttgattacgttgccaacttggttattgctgc
tggtgctaaatcttctaagtctactgaattgaaggtctacaactgctgttcttctgcttgtaacccaattactatcggtaagttgatgtcc
atgtttgctgaagatgctatcaagcaaaagtcttacgctatgccattgccaggttggtacatttttactaagtacaagtggttggtctt
gttgttgaccattttgttccaagttattccagcctacattaccgacttgtacagacatttgattggtaagaacccaagatatatcaagtt
gcaatccttggtcaatcaaaccagatcctccattgatttcttcacctctcattcttgggttatgaaggctgatagagtcagagaattatt
cgcttctttgtctccagcagataagtacttgtttccatgtgatccaaccgatattaactggacccattacattcaagattactgctggg
gtgttagacatttcttggaaaaaaaaagctacgaataa
SEQ ID NO: 8-H. armigera fatty acyl reductase
MVVLTSKETKPSVAEFYAGKSVFITGGTGFLGKVFIEKLLYSCPDIGNIYMLIREKKGLS
VSERIKHFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
RYVKDGISEEETEKILNGRPNTYTFTKALTEHLVAENQAYVPTIIVRPSVVAA
IKDEPIKGWLGNWYGATGLTVFTAKGLNRVIYGHSSNIVDLIPVDYVANLVIAAGAKSSKST
ELKVYNCCSSACNPITIGKLMSMFAEDAIKQKSYAMPLPGWYIFTKYKWLVLLLTILFQVIPA
YITDLYRHLIGKNPRYIKLQSLVNQTRSSIDFFTSHSWVMKADRVRELFASLSPADKYLFPC
DPTDINWTHYIQDYCWGVRHFLEKKSYE
SEQ ID NO: 9-S. cerevisiae-codon-optimized nucleotide sequence of H. armigera fatty
acyl reductase with signal peptide changed to HDEL; mRNA-coding sequence.
atggttgtcttgacctccaaagaaactaagccatctgttgctgaattttacgctggtaagtctgttttcattactggtggtactggtttctt
gggtaaggttttcattgaaaagttgttgtactcctgcccagatatcggtaatatctacatgttgatcagagaaaagaagggtttgtcc
gtttccgaaagaatcaagcactttttggatgatcctttgttcaccagattgaaagaaaaaagaccagccgacttggaaaagatcg
ttttgattccaggtgatattactgctccagatttgggtattacctccgaaaacgaaaagatgttgatcgaaaaggtcagtgtcattatt
cattctgctgctaccgttaagttcaacgaaccattgccaactgcttggaagattaacgttgaaggtactagaatgatgttggccttgt
ctagaagaatgaagagaatcgaagttttcatccatatctctaccgcttacactaacaccaacagagaagttgttgacgaaatctt
gtatccagctccagctgatattgatcaagttcacagatatgttaaggacggtatctctgaagaagaaactgaaaaaatcttgaac
ggtagaccaaacacttacactttcactaaggctttgaccgaacatttggttgctgaaaatcaagcttacgttccaaccattatcgtta
gaccatcagttgttgctgccattaaggatgaacctattaagggttggttgggtaattggtatggtgctacaggtttgactgtttttactg
ctaagggtttgaacagagttatctacggtcactcttctaacatcgttgatttgatcccagttgattacgttgccaacttggttattgctgc
tggtgctaaatcttctaagtctactgaattgaaggtctacaactgctgttcttctgcttgtaacccaattactatcggtaagttgatgtcc
atgtttgctgaagatgctatcaagcaaaagtcttacgctatgccattgccaggttggtacatttttactaagtacaagtggttggtctt
gttgttgaccattttgttccaagttattccagcctacattaccgacttgtacagacatttgattggtaagaacccaagatatatcaagtt
gcaatccttggtcaatcaaaccagatcctccattgatttcttcacctctcattcttgggttatgaaggctgatagagtcagagaattatt
cgcttctttgtctccagcagataagtacttgtttccatgtgatccaaccgatattaactggacccattacattcaagattactgctggg
gtgttagacatttcttggaacatgatgaattgtaa
SEQ ID NO: 10-H. armigera fatty acyl reductase with signal peptide changed to HDEL
MVVLTSKETKPSVAEFYAGKSVFITGGTGFLGKVFIEKLLYSCPDIGNIYMLIREKKGLS
VSERIKHFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
RYVKDGISEEETEKILNGRPNTYTFTKALTEHLVAENQAYVPTIIVRPSVVAAIKDEPIKGWL
GNWYGATGLTVFTAKGLNRVIYGHSSNIVDLIPVDYVANLVIAAGAKSSKSTELKVYNCCSS
ACNPITIGKLMSMFAEDAIKQKSYAMPLPGWYIFTKYKWLVLLLTILFQVIPAYITDLYRHLIG
KNPRYIKLQSLVNQTRSSIDFFTSHSWVMKADRVRELFASLSPADKYLFPCDPTDINWTHYI
QDYCWGVRHFLEHDEL
SEQ ID NO: 11-S. cerevisiae-codon-optimized nucleotide sequence of H. assulta fatty
acyl reductase; mRNA-coding sequence.
atggttgtcttgacctccaaagaaactaagccatctgttgctgaattttacgctggtaagtctgttttcattactggtggtactggtttctt
gggtaagatcttcattgaaaagttgttgtactcctgcccagatatcggtaatatctacatgttgatcagagaaaagaagggtttgtc
cgtttccgaaagaatcaagcaatttttggatgaccctttgttcaccagattgaaagaaaaaagaccagccgacttggaaaagat
cgttttgattccaggtgatattactgctccagatttgggtattacctccgaaaacgaaaagatgttgatcgaaaaggtcagtgtcatt
attcattctgctgctaccgttaagttcaacgaaccattgccaactgcttggaagattaacgttgaaggtactagaatgatgttggcct
tgtctagaagaatgaagagaatcgaagttttcatccatatctctaccgcttacactaacaccaacagagaagttgttgacgaaat
cttgtatccagctccagctgatattgatcaagttcaccaatatgttaaggacggtatctctgaagaagaaactgaaaaaatcttga
acggtagaccaaacacttacactttcactaaggctttgaccgaacatttggttgctgaaaatcaagcttacgttccaaccattatcg
ttagaccatcagttgttgctgccattaaggatgaacctattaagggttggttgggtaattggtatggtgctacaggtttgactgtttttac
tgctaagggtttgaacagagttatctacggtcattcctcttacatcgttgatttgatcccagttgattacgttgccaacttggttattgctg
ctggtgctaaatcttctaagtctactgaattgaaggtctacaactgctgttcttctgcttgtaacccaattactatcggtaagttgatgtc
catgtttgctgaagatgctatcaagcaaaagtcttacgctatgccattgccaggttggtatgtttttacaaagtacaagtggttggtct
tgttgttgaccattttgttccaagttattccagcctacattaccgacttgtacagacatttgattggtaagaacccaagatatatcaagt
tgcaatccttggtcaatcaaaccagatcctccattgatttcttcacctctcattcttgggttatgaaggctgatagagtcagagaattat
tcgcttctttgtctccagcagataagtacttgtttccatgtgatccaaccgatattaactggacccattacattcaagattactgctggg
gtgttagacacttcttggaaaaaaagactaccaacaagtaa
SEQ ID NO: 12-Amino acid sequence of H. assulta fatty acyl reductase
MVVLTSKETKPSVAEFYAGKSVFITGGTGFLGKIFIEKLLYSCPDIGNIYMLIREKKGLS
VSERIKQFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
QYVKDGISEEETEKILNGRPNTYTFTKALTEHLVAENQAYVPTIIVRPSVVAAIKDEPIKGWL
GNWYGATGLTVFTAKGLNRVIYGHSSYIVDLIPVDYVANLVIAAGAKSSKSTELKVYNCCSS
ACNPITIGKLMSMFAEDAIKQKSYAMPLPGWYVFTKYKWLVLLLTILFQVIPAYITDLYRHLIG
KNPRYIKLQSLVNQTRSSIDFFTSHSWVMKADRVRELFASLSPADKYLFPCDPTDINWTHYI
QDYCWGVRHFLEKKTTNK
SEQ ID NO: 13-S. cerevisiae-codon-optimized nucleotide sequence of H. assulta fatty
acyl reductase with signal peptide changed to HDEL; mRNA-coding sequence.
atggttgtcttgacctccaaagaaactaagccatctgttgctgaattttacgctggtaagtctgttttcattactggtggtactggtttctt
gggtaagatcttcattgaaaagttgttgtactcctgcccagatatcggtaatatctacatgttgatcagagaaaagaagggtttgtc
cgtttccgaaagaatcaagcaatttttggatgaccctttgttcaccagattgaaagaaaaaagaccagccgacttggaaaagat
cgttttgattccaggtgatattactgctccagatttgggtattacctccgaaaacgaaaagatgttgatcgaaaaggtcagtgtcatt
attcattctgctgctaccgttaagttcaacgaaccattgccaactgcttggaagattaacgttgaaggtactagaatgatgttggcct
tgtctagaagaatgaagagaatcgaagttttcatccatatctctaccgcttacactaacaccaacagagaagttgttgacgaaat
cttgtatccagctccagctgatattgatcaagttcaccaatatgttaaggacggtatctctgaagaagaaactgaaaaaatcttga
acggtagaccaaacacttacactttcactaaggctttgaccgaacatttggttgctgaaaatcaagcttacgttccaaccattatcg
ttagaccatcagttgttgctgccattaaggatgaacctattaagggttggttgggtaattggtatggtgctacaggtttgactgtttttac
tgctaagggtttgaacagagttatctacggtcattcctcttacatcgttgatttgatcccagttgattacgttgccaacttggttattgctg
ctggtgctaaatcttctaagtctactgaattgaaggtctacaactgctgttcttctgcttgtaacccaattactatcggtaagttgatgtc
catgtttgctgaagatgctatcaagcaaaagtcttacgctatgccattgccaggttggtatgtttttacaaagtacaagtggttggtct
tgttgttgaccattttgttccaagttattccagcctacattaccgacttgtacagacatttgattggtaagaacccaagatatatcaagt
tgcaatccttggtcaatcaaaccagatcctccattgatttcttcacctctcattcttgggttatgaaggctgatagagtcagagaattat
tcgcttctttgtctccagcagataagtacttgtttccatgtgatccaaccgatattaactggacccattacattcaagattactgctggg
gtgttagacacttcttggaacatgatgaattgtaa
SEQ ID NO: 14-amino acid sequence of H. assulta fatty acyl reductase with signal
peptide changed to HDEL
MVVLTSKETKPSVAEFYAGKSVFITGGTGFLGKIFIEKLLYSCPDIGNIYMLIREKKGLS
VSERIKQFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
QYVKDGISEEETEKILNGRPNTYTFTKALTEHLVAENQAYVPTIIVRPSVVAAIKDEPIKGWL
GNWYGATGLTVFTAKGLNRVIYGHSSYIVDLIPVDYVANLVIAAGAKSSKSTELKVYNCCSS
ACNPITIGKLMSMFAEDAIKQKSYAMPLPGWYVFTKYKWLVLLLTILFQVIPAYITDLYRHLIG
KNPRYIKLQSLVNQTRSSIDFFTSHSWVMKADRVRELFASLSPADKYLFPCDPTDINWTHYI
QDYCWGVRHFLEHDEL
SEQ ID NO: 15-S. cerevisiae-codon-optimized nucleotide sequence of H. subflexa fatty
acyl reductase; mRNA-coding sequence.
atggttgtcttgacctccaaagaaactaagccatctgttgctgaattttacgctggtaagtctgttttcattactggtggtactggtttctt
gggtaaggttttcattgaaaagttgttgtactcctgcccagatatcggtaatatctacatgttgatcagagaaaagaagggtttgtcc
gtttccgaaagaatcaagcactttttggatgatcctttgttcaccagattgaaagaaaaaagaccagccgacttggaaaagatcg
ttttgattccaggtgatattactgctccagatttgggtattacctccgaaaacgaaaagatgttgatcgaaaaggtcagtgtcattatt
cattctgctgctaccgttaagttcaacgaaccattgccaactgcttggaagattaacgttgaaggtactagaatgatgttggccttgt
ctagaagaatgaagagaatcgaagttttcatccatatctctaccgcttacactaacaccaacagagaagttgttgacgaaatctt
gtatccagctccagctgatattgatcaagttcaccaatatgttaaggacggtatctctgaagaagaaactgaaaaaatcttgaac
ggtagaccaaacacttacactttcactaaggctttgaccgaacatttggttgctgaaaatcaagcttacgttccaaccattatcgtta
gaccatcagttgttgctgccattaaggatgaacctattaagggttggttgggtaattggtatggtgctacaggtttgactgtttttactg
ctaagggtttgaacagagttatctacggtcactcttctaacatcgttgatttgatcccagttgattacgttgccaacttggttattgctgc
tggtgctaaatcttctaagtctactgaattgaaggtctacaactgctgttcttctgcttgtaacccaattactatcggtaagttgatgtcc
atgtttgctgaagatgctatcaagcaaaagtcttacgctatgccattgccaggttggtacatttttactaagtacaagtggttggtctt
gttgttgaccattttgttccaagttattccagcctacattaccgacttgtacagacatttgattggtaagaacccaagatatatcaagtt
gcaatccttggtcaatcaaaccagatcctccattgatttcttcaccaaccattcttgggttatgaaggctgatagagtcagagaatt
attcgcttctttgtctccagcagataagtacttgtttccatgtgatccagtcaacatcaattggagacaatatatccaagattactgctg
gggtgttagacatttcttggaaaaaaagacttaa
SEQ ID NO: 16-Amino acid of H. subflexa fatty acyl reductase
MVVLTSKETKPSVAEFYAGKSVFITGGTGFLGKVFIEKLLYSCPDIGNIYMLIREKKGLS
VSERIKHFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
QYVKDGISEEETEKILNGRPNTYTFTKALTEHLVAENQAYVPTIIVRPSVVAA
IKDEPIKGWLGNWYGATGLTVFTAKGLNRVIYGHSSNIVDLIPVDYVANLVIAAGAKSSKST
ELKVYNCCSSACNPITIGKLMSMFAEDAIKQKSYAMPLPGWYIFTKYKWLVLLLTILFQVIPA
YITDLYRHLIGKNPRYIKLQSLVNQTRSSIDFFTNHSWVMKADRVRELFASLSPADKYLFPC
DPVNINWRQYIQDYCWGVRHFLEKKT
SEQ ID NO: 17-S. cerevisiae-codon-optimized nucleotide sequence of H. subflexa fatty
acyl reductase with signal peptide changed to HDEL; mRNA-coding sequence.
atggttgtcttgacctccaaagaaactaagccatctgttgctgaattttacgctggtaagtctgttttcattactggtggtactggtttctt
gggtaaggttttcattgaaaagttgttgtactcctgcccagatatcggtaatatctacatgttgatcagagaaaagaagggtttgtcc
gtttccgaaagaatcaagcactttttggatgatcctttgttcaccagattgaaagaaaaaagaccagccgacttggaaaagatcg
ttttgattccaggtgatattactgctccagatttgggtattacctccgaaaacgaaaagatgttgatcgaaaaggtcagtgtcattatt
cattctgctgctaccgttaagttcaacgaaccattgccaactgcttggaagattaacgttgaaggtactagaatgatgttggccttgt
ctagaagaatgaagagaatcgaagttttcatccatatctctaccgcttacactaacaccaacagagaagttgttgacgaaatctt
gtatccagctccagctgatattgatcaagttcaccaatatgttaaggacggtatctctgaagaagaaactgaaaaaatcttgaac
ggtagaccaaacacttacactttcactaaggctttgaccgaacatttggttgctgaaaatcaagcttacgttccaaccattatcgtta
gaccatcagttgttgctgccattaaggatgaacctattaagggttggttgggtaattggtatggtgctacaggtttgactgtttttactg
ctaagggtttgaacagagttatctacggtcactcttctaacatcgttgatttgatcccagttgattacgttgccaacttggttattgctgc
tggtgctaaatcttctaagtctactgaattgaaggtctacaactgctgttcttctgcttgtaacccaattactatcggtaagttgatgtcc
atgtttgctgaagatgctatcaagcaaaagtcttacgctatgccattgccaggttggtacatttttactaagtacaagtggttggtctt
gttgttgaccattttgttccaagttattccagcctacattaccgacttgtacagacatttgattggtaagaacccaagatatatcaagtt
gcaatccttggtcaatcaaaccagatcctccattgatttcttcaccaaccattcttgggttatgaaggctgatagagtcagagaatt
attcgcttctttgtctccagcagataagtacttgtttccatgtgatccagtcaacatcaattggagacaatatatccaagattactgctg
gggtgttagacatttcttgcatgatgaattgtaa
SEQ ID NO: 18-amino acid sequence of H. subflexa fatty acyl reductase with signal
peptide changed to HDEL
MVVLTSKETKPSVAEFYAGKSVFITGGTGFLGKVFIEKLLYSCPDIGNIYMLIREKKGLS
VSERIKHFLDDPLFTRLKEKRPADLEKIVLIPGDITAPDLGITSENEKMLIEKVSVIIHSAATVK
FNEPLPTAWKINVEGTRMMLALSRRMKRIEVFIHISTAYTNTNREVVDEILYPAPADIDQVH
QYVKDGISEEETEKILNGRPNTYTFTKALTEHLVAENQAYVPTIIVRPSVVAA
IKDEPIKGWLGNWYGATGLTVFTAKGLNRVIYGHSSNIVDLIPVDYVANLVIAAGAKSSKST
ELKVYNCCSSACNPITIGKLMSMFAEDAIKQKSYAMPLPGWYIFTKYKWLVLLLTILFQVIPA
YITDLYRHLIGKNPRYIKLQSLVNQTRSSIDFFTNHSWVMKADRVRELFASLSPADKYLFPC
DPVNINWRQYIQDYCWGVRHFLHDEL
SEQ ID NO: 34-Sc_FAA1 DNA sequence; mRNA-coding sequence.
ATGGTTGCTCAATATACCGTTCCAGTTGGGAAAGCCGCCAATGAGCATGAAACTGCTC
CAAGAAGAAATTATCAATGCCGCGAGAAGCCGCTCGTCAGACCGCCTAACACAAAGTG
TTCCACTGTTTATGAGTTTGTTCTAGAGTGCTTTCAGAAGAACAAAAATTCAAATGCTAT
GGGTTGGAGGGATGTTAAGGAAATTCATGAAGAATCCAAATCGGTTATGAAAAAAGTT
GATGGCAAGGAGACTTCAGTGGAAAAGAAATGGATGTATTATGAACTATCGCATTATCA
TTATAATTCATTTGACCAATTGACCGATATCATGCATGAAATTGGTCGTGGGTTGGTGA
AAATAGGATTAAAGCCTAATGATGATGACAAATTACATCTTTACGCAGCCACTTCTCAC
AAGTGGATGAAGATGTTCTTAGGAGCGCAGTCTCAAGGTATTCCTGTCGTCACTGCCT
ACGATACTTTGGGAGAGAAAGGGCTAATTCATTCTTTGGTGCAAACGGGGTCTAAGGC
CATTTTTACCGATAACTCTTTATTACCATCCTTGATCAAACCAGTGCAAGCCGCTCAAG
ACGTAAAATACATAATTCATTTCGATTCCATCAGTTCTGAGGACAGGAGGCAAAGTGGT
AAGATCTATCAATCTGCTCATGATGCCATCAACAGAATTAAAGAAGTTAGACCTGATAT
CAAGACCTTTAGCTTTGACGACATCTTGAAGCTAGGTAAAGAATCCTGTAACGAAATCG
ATGTTCATCCACCTGGC
AAGGATGATCTTTGTTGCATCATGTATACGTCTGGTTCTACAGGTGAGCCAAAGGGTG
TTGTCTTGAAACATTCAAATGTTGTCGCAGGTGTTGGTGGTGCAAGTTTGAATGTTTTG
AAGTTTGTGGGCAATACCGACCGTGTTATCTGTTTTTTGCCACTAGCTCATATTTTTGA
ATTGGTTTTCGAACTATTGTCCTTTTATTGGGGGGCCTGCATTGGTTATGCCACCGTAA
AAACTTTAACTAGCAGCTCTGTGAGAAATTGTCAAGGTGATTTGCAAGAATTCAAGCCC
ACAATCATGGTTGGTGTCGCCGCTGTTTGGGAAACAGTGAGAAAAGGGATCTTAAACC
AAATTGATAATTTGCCCTTCCTCACCAAGAAAATCTTCTGGACCGCGTATAATACCAAG
TTGAACATGCAACGTCTCCACATCCCTGGTGGCGGCGCCTTAGGAAACTTGGTTTTCA
AAAAAATCAGAACTGCCACAGGTGGCCAATTAAGATATTTGTTAAACGGTGGTTCTCCA
ATCAGTCGGGATGCTCAGGAATTCATCACAAATTTAATCTGCCCTATGCTTATTGGTTA
CGGTTTAACCGAGACATGCGCTAGTACCACCATCTTGGATCCTGCTAATTTTGAACTC
GGCGTCGCTGGTGACCTAACAGGTTGTGTTACCGTCAAACTAGTTGATGTTGAAGAAT
TAGGTTATTTTGCTAAAAACAACCAAGGTGAAGTTTGGATCACAGGTGCCAATGTCACG
CCTGAATATTATAAGAATGAGGAAGAAACTTCTCAAGCTTTAACAAGCGATGGTTGGTT
CAAGACCGGTGACATCGGTGAATGGGAAGCAAATGGCCATTTGAAAATAATTGACAGG
AAGAAAAACTTGGTCAAAACAATGAACGGTGAATATATCGCACTCGAGAAATTAGAGTC
CGTTTACAGATCTAACGAATATGTTGCTAACATTTGTGTTTATGCCGACCAATCTAAGA
CTAAGCCAGTTGGTATTATTGTACCAAATCATGCTCCATTAACGAAGCTTGCTAAAAAG
TTGGGAATTATGGAACAAAAAGACAGTTCAATTAATATCGAAAATTATTTGGAGGATGC
AAAATTGATTAAAGCTGTTTATTCTGATCTTTTGAAGACAGGTAAAGACCAAGGTTTGG
TTGGCATTGAATTACTAGCAGGCATAGTGTTCTTTGACGGCGAATGGACTCCACAAAA
CGGTTTTGTTACGTCCGCTCAGAAATTGAAAAGAAAAGACATTTTGAATGCTGTCAAAG
ATAAAGTTGACGCCGTTTATAGTTCGTCTTAA
SEQ ID NO: 35-Sc_FAA1 amino acid sequence
MVAQYTVPVGKAANEHETAPRRNYQCREKPLVRPPNTKCSTVYEFVLECFQKNKNSNAM
GWRDVKEIHEESKSVMKKVDGKETSVEKKWMYYELSHYHYNSFDQLTDIMHEIGRGLVKI
GLKPNDDDKLHLYAATSHKWMKMFLGAQSQGIPVVTAYDTLGEKGLIHSLVQTGSKAIFTD
NSLLPSLIKPVQAAQDVKYIIHFDSISSEDRRQSGKIYQSAHDAINRIKEVRPDIKTFSFDDILK
LGKESCNEIDVHPPGKDDLCCIMYTSGSTGEPKGVVLKHSNVVAGVGGASLNVLKFVGNT
DRVICFLPLAHIFELVFELLSFYWGACIGYATVKTLTSSSVRNCQGDLQEFKPTIMVGVAAV
WETVRKGILNQIDNLPFLTKKIFWTAYNTKLNMQRLHIPGGGALGNLVFKKIRTATGGQLRY
LLNGGSPISRDAQEFITNLICPMLIGYGLTETCASTTILDPANFELGVAGDLTGCVTVKLVDV
EELGYFAKNNQGEVWITGANVTPEYYKNEEETSQALTSDGWFKTGDIGEWEANGHLKIID
RKKNLVKTMNGEYIALEKLESVYRSNEYVANICVYADQSKTKPVGIIVPNHAPLTKLAKKLGI
MEQKDSSINIENYLEDAKLIKAVYSDLLKTGKDQGLVGIELLAGIVFFDGEWTPQNGFVTSA
QKLKRKDILNAVKDKVDAVYSSS
SEQ ID NO: 36-Yl_FAA DNA sequence; mRNA-coding sequence.

| | atggtcggat acacaatttc ctcaaagccc gtgtcggtgg aggtcggccc cgccaagcct |
|---|---|
| 61 | ggcgagactg ccccccgacg aaacgtcatt gccaaggacg cccctgtcgt cttccccgac |
| 121 | aacgactcgt ccctgaccac cgtctacaag ctgttcaaaa agtacgccga gatcaacagc |
| 181 | gagcgaaagg ccatgggatg gcgagacacc atcgacatcc acgtggagac caaacaggtg |
| 241 | accaaggtcg tggacggagt ggagaagaag gtgcccaagg aatggaagta ctttgagatg |
| 301 | ggcccttaca agtggctctc atacaaggag gcccttaagc tggtccatga ttatggagct |
| 361 | ggtcttcgac acctcggaat caagcccaag gagaagatgc acatttacgc ccagacctcc |
| 421 | caccgatgga tgctctctgg cctggcttct ctgtctcagg gtattcccat tgtcactgcc |
| 481 | tacgacactc ttggagagga gggtctcact cgatctctcc aggagaccaa ctcggtcatc |
| 541 | atgtttaccg acaaggctct gctgagctct ctcaaggtct ctctcaagaa gggcaccgat |
| 601 | ctgcgaatca tcatctacgg aggtgatctg acccccgacg acaagaaggc cggaaacacg |
| 661 | gagattgacg ccatcaagga gattgttcca gatatgaaga tctacaccat ggacgaggtt |
| 721 | gtcgctctcg gccgagaaca cccccacccc gtggaggagg tcgactatga ggacctggcc |
| 781 | ttcatcatgt acacctctgg ttctaccggt gtccccaagg gtgtggttct gcagcacaag |
| 841 | cagatcctcg cctctgtggc cggtgtcacc aagatcattg accgatctat catcggcaac |
| 901 | acagaccggc ttctcaactt cctgcccctc gcacacattt tcgagtttgt gttcgagatg |
| 961 | gtcaccttct ggtggggtgc ttctctgggt tacggaaccg tcaagaccat ttccgatctg |
| 1021 | tccatgaaga actgtaaggg agacattcga gagctcaagc ccaccatcat ggtcggcgtt |
| 1081 | cccgctgtct gggaacctat gcgaaagggt attcttggca agatcaagga gctgtctcct |
| 1141 | ctgatgcagc gggtcttctg ggcctcattt gccgccaagc agcgtctcga cgagaacgga |
| 1201 | ctccctggtg gatctatcct cgactcgctc attttcaaga aggtcaagga cgccactgga |
| 1261 | ggctgtctcc gatacgtgtg taacggaggt gctccagtat ctgtcgacac ccagaagttc |
| 1321 | atcaccactc tcatctgtcc catgctgatt ggatgcggtc tgaccgagac tacagccaac |
| 1381 | accaccatca tgtcgcctaa atcgtacgcc tttggcacca ttggtgagcc caccgccgcc |
| 1441 | gtgaccctca agctcattga cgtgcctgaa gccggctact tcgccgagaa caaccaggga |
| 1501 | gagctgtgca tcaagggcaa cgtcgtgatg aaggagtact acaagaacga ggaggagacc |
| 1561 | aagaaggcgt tctccgacga tggctatttc ctcaccggtg atattgccga gtggaccgcc |
| 1621 | aatggccagc tcagaatcat tgaccgacga aagaacctcg tcaagaccca gaacggagag |
| 1681 | tacattgctc tggagaagct cgagacacag taccgatcgt cgtcgtacgt ggccaacctg |
| 1741 | tgtgtgtacg ccgaccagaa ccgagtcaag cccattgctc tggtcattcc taacgagggc |
| 1801 | cccaccaaga agcttgccca gagcttgggc gtcgattctg acgactggga cgccgtctgt |
| 1861 | tccaacaaaa aggtggtcaa ggctgtgctc aaggacatgc tcgataccgg ccgatctctg |
| 1921 | ggtctgtccg gcattgagct gctgcaaggc attgtgttgc tgcctggcga gtggactcct |
| 1981 | cagaacagct acctgactgc tgcccagaag ctcaaccgaa agaagattgt ggatgataac |
| 2041 | aagaaggaaa ttgatgagtg ctacgagcag tcttag |

SEQ ID NO: 37-Yl_FAA amino acid sequence
MVGYTISSKPVSVEVGPAKPGETAPRRNVIAKDAPVVFPDNDSSLTTVYKLFKKYAEINSE
RKAMGWRDTIDIHVETKQVTKVVDGVEKKVPKEWKYFEMGPYKWLSYKEALKLVHDYGA
GLRHLGIKPKEKMHIYAQTSHRWMLSGLASLSQGIPIVTAYDTLGEEGLTRSLQETNSVIMF
TDKALLSSLKVSLKKGTDLRIIIYGGDLTPDDKKAGNTEIDAIKEIVPDMKIYTMDEVVALGRE
HPHPVEEVDYEDLAFIMYTSGSTGVPKGVVLQHKQILASVAGVTKIIDRSIIGNTDRLLNFLP
LAHIFEFVFEMVTFWWGASLGYGTVKTISDLSMKNCKGDIRELKPTIMVGVPAVWEPMRK
GILGKIKELSPLMQRVFWASFAAKQRLDENGLPGGSILDSLIFKKVKDATGGCLRYVCNGG
APVSVDTQKFITTLICPMLIGCGLTETTANTTIMSPKSYAFGTIGEPTAAVTLKLIDVPEAGYF
AENNQGELCIKGNVVMKEYYKNEEETKKAFSDDGYFLTGDIAEWTANGQLRIIDRRKNLVK
TQNGEYIALEKLETQYRSSSYVANLCVYADQNRVKPIALVIPNEGPTKKLQSLGVDSDDWD
AVCSNKKVVKAVLKDMLDTGRSLGLSGIELLQGIVLLPGEWTPQNSYLTAAQKLNRKKIVD
DNKKEIDECYEQS
SEQ ID NO: 38: Saccharomyces cerevisiae ATF1 DNA sequence; mRNA-coding
sequence.
ATGAATGAAA TCGATGAGAA AAATCAGGCC CCCGTGCAAC AAGAATGCCT
GAAAGAGATG ATTCAGAATG GGCATGCTCG GCGTATGGGA TCTGTTGAAG
ATCTGTATGT TGCTCTCAAC AGACAAAACT TATATCGAAA CTTCTGCACA
TATGGAGAAT TGAGTGATTA CTGTACTAGG GATCAGCTCA CATTAGCTTT
GAGGGAAATC TGCCTGAAAA ATCCAACTCT TTTACATATT GTTCTACCAA
CAAGATGGCC AAATCATGAA AATTATTATC GCAGTTCCGA ATACTATTCA
CGGCCACATC CAGTGCATGA TTATATTTCA GTATTACAAG AATTGAAACT
GAGTGGTGTG GTTCTCAATG AACAACCTGA GTACAGTGCA GTAATGAAGC
AAATATTAGA AGAATTCAAA AATAGTAAGG GTTCCTATAC TGCAAAAATT
TTTAAACTTA CTACCACTTT GACTATTCCT TACTTTGGAC CAACAGGACC
GAGTTGGCGG CTAATTTGTC TTCCAGAAGA GCACACAGAA AAGTGGAAAA
AATTTATCTT TGTATCTAAT CATTGCATGT CTGATGGTCG GTCTTCGATC
CACTTTTTTC ATGATTTAAG AGACGAATTA AATAATATTA AAACTCCACC
AAAAAAATTA GATTACATTT TCAAGTACGA GGAGGATTAC CAATTATTGA
GGAAACTTCC AGAACCGATC GAAAAGGTGA TAGACTTTAG ACCACCGTAC
TTGTTTATTC CGAAGTCACT TCTTTCGGGT TTCATCTACA ATCATTTGAG
ATTTTCTTCA AAAGGTGTCT GTATGAGAAT GGATGATGTG GAAAAAACCG
ATGATGTTGT CACCGAGATC ATCAATATTT CACCAACAGA ATTTCAAGCG
ATTAAAGCAA ATATTAAATC AAATATCCAA GGTAAGTGTA CTATCACTCC
GTTTTTACAT GTTTGTTGGT TTGTATCTCT TCATAAATGG GGTAAATTTT
TCAAACCATT GAACTTCGAA TGGCTTACGG ATATTTTTAT CCCCGCAGAT
TGCCGCTCAC AACTACCAGA TGATGATGAA ATGAGACAGA TGTACAGATA
TGGCGCTAAC GTTGGATTTA TTGACTTCAC CCCCTGGATA AGCGAATTTG
ACATGAATGA TAACAAAGAA AATTTTTGGC CACTTATTGA GCACTACCAT
GAAGTAATTT CGGAAGCTTT AAGAAATAAA AAGCATCTCC ATGGCTTAGG
GTTCAATATA CAAGGCTTCG TTCAAAAATA TGTGAACATT GACAAGGTAA
TGTGCGATCG TGCCATCGGG AAAAGACGCG GAGGTACATT GTTAAGCAAT
GTAGGTCTGT TTAATCAGTT AGAGGAGCCC GATGCCAAAT ATTCTATATG
CGATTTGGCA TTTGGCCAAT TTCAAGGATC CTGGCACCAA GCATTTTCCT
TGGGTGTTTG TTCGACTAAT GTAAAGGGGA TGAATATTGT TGTTGCTTCA
ACAAAGAATG TTGTTGGTAG TCAAGAATCT CTCGAAGAGC TTTGCTCCAT
TTACAAAGCT CTCCTTTTAG GCCCTTAG
SEQ ID NO: 39: Saccharomyces cerevisiae Atf1 amino acid sequence
MNEIDEKNQAPVQQECLKEMIQNGHARRMGSVEDLYVALNRQNLYRNFCTYGELSDYCT
RDQLTLALREICLKNPTLLHIVLPTRWPNHENYYRSSEYYSRPHPVHDYISVLQELKLSGVV
LNEQPEYSAVMKQILEEFKNSKGSYTAKIFKLTTTLTIPYFGPTGPSWRLICLPEEHTEKWK
KFIFVSNHCMSDGRSSIHFFHDLRDELNNIKTPPKKLDYIFKYEEDYQLLRKLPEPIEKVIDF
RPPYLFIPKSLLSGFIYNHLRFSSKGVCMRMDDVEKTDDVVTEIINISPTEFQAIKANIKSNIQ
GKCTITPFLHVCWFVSLHKWGKFFKPLNFEWLTDIFIPADCRSQLPDDDEMRQMYRYGAN
VGFIDFTPWISEFDMNDNKENFWPLIEHYHEVISEALRNKKHLHGLGFNIQGFVQKYVNIDK
VMCDRAIGKRRGGTLLSNVGLFNQLEEPDAKYSICDLAFGQFQGSWHQAFSLGVCSTNV
KGMNIVVASTKNVVGSQESLEELCSIYKALLLGP
SEQ ID NO: 40: Sl_Δ11-desaturase DNA sequence; mRNA-coding sequence.
GGACACTGACATGGACTGAAGGAGTAGAGAATCGGCCCGTGGAGTTGGCCTTCATTT
TCAGTCTTATCTCTCGGTGTTATGGTAGTCACTTATATCGGTATTAAAATAAGTGAATAA
GGCTTGTAAAAATGGCGCAATGTGTACAAACAACAACGATTTTGGAACAAAAAGAAGA
GAAAACAGTAACTTTGCTGGTACCTCAAGCGGGAAAGAGGAAGTTTGAAATTGTGTAT
TTTAATATCATCACCTTCGCTTACTGGCATATAGCTGGACTATATGGCCTTTATTTGTGC
TTCACTTCAACAAAATGGGCGACAGTTTTATTCTCATTCTTTCTATTCGTCGTAGCAGAA
GTAGGGGTCACGGCTGGCTCCCACAGACTTTGGTCGCATAAAACTTACAAAGCAAAAC
TACCTTTACAAATTCTGCTAATGGTGATGAATTCCCTTGCATTTCAAAACACAGTCATTG
ATTGGGTGAGAGACCATCGACTCCATCATAAGTATAGCGACACTGATGCCGATCCCCA
TAATGCCTCCCGAGGATTTTTCTATTCGCACGTCGGTTGGCTGCTTGTGAGAAAACAC
CCTGATGTCAAGAAACGAGGAAAGGAAATTGATATATCTGATATTTACAACAATCCGGT
ACTGAGGTTCCAGAAGAAGTACGCAATTCCTTTCATCGGGGCAGTTTGTTTCGTCTTAC
CAACATTGATACCGGTTTACGGTTGGGGAGAAACCTGGACTAATGCCTGGCACGTCG
CCATGCTGCGGTACATTATGAACCTTAACGTCACCTTCCTGGTCAACAGCGCTGCTCA
TATATATGGAAAGAGACCTTATGACAAGAAGATCCTACCATCTCAAAACATAGCTGTGT
CCATTGCAACCTTTGGGGAAGGTTTCCATAATTATCATCATGTATTTCCATGGGATTAT
CGCGCAGCTGAACTTGGAAATAACAGTTTGAATTTCCCTACGAAATTTATTGATTTCTTT
GCGTGGATCGGATGGGCGTATGACCTAAAGACTGTTTCGAAAGAAATGATAAAACAAA
GGTCAAAAAGAACTGGTGATGGAACTAATCTATGGGGGTTAGAAGATGTGGATACCCC
GGAGGATTTAAAAAATACAAAAGGCGAATAGGCAAACCCTTAAACTCAAACAGTGAGG
TTTAATGTGATATTTAGAATTAGAATTAATTTATTTGAAATTAAATGAAGGTTTTGGATAA
CTGTTTTTAATAATAAAAATAGTTTTTCGATTAAATTCCTTAGATTATTTTAAAGGAAATG
TATAAGGTACTCGCGTGGTTAGCAACCCAGCAGTCCCTGTTTATCTGTTTTTATGAATT
TATTCTATGAATGTAGATGTCGCATGAAATTTTAAAATGTTGCATTTGTATAATTTTACTT
ATGAATAAATAAATTTATTTTTAAAAAAAAAAAAAAAAAAAAAAAAAAAAAAAAAA
SEQ ID NO: 41: Sl_Δ11-desaturase amino acid sequence
MAQCVQTTTILEQKEEKTVTLLVPQAGKRKFEIVYFNIITFAYWHIAGLYGLYLCFTSTK
WATVLFSFFLFVVAEVGVTAGSHRLWSHKTYKAKLPLQILLMVMNSLAFQNTVIDWVRDH
RLHHKYSDTDADPHNASRGFFYSHVGWLLVRKHPDVKKRGKEIDISDIYNNPVLRFQKKY
AIPFIGAVCFVLPTLIPVYGWGETWTNAWHVAMLRYIMNLNVTFLVNSAAHIYGKRPYDKKI
LPSQNIAVSIATFGEGFHNYHHVFPWDYRAAELGNNSLNFPTKFIDFFAWIGWAYDLKTVS
KEMIKQRSKRTGDGTNLWGLEDVDTPEDLKNTKGE
SEQ ID NO: 42: As_Δ11-desaturase DNA sequence; mRNA-coding sequence.
ATGGCTCAAGGTGTCCAAACAACTACGATATTGAGGGAGGAAGAGCCGTCATTGACTT
TCGTGGTACCTCAAGAACCGAGAAAGTATCAAATCGTGTACCCAAACCTTATCACATTT
GGGTACTGGCATATAGCTGGTTTATACGGGCTATATTTGTGCTTTACTTCGGCAAAATG
GCAAACAATTTTATTCAGTTTCATGCTCGTTGTGTTAGCAGAGTTGGGAATAACAGCCG
GCGCTCACAGGTTATGGGCCCACAAAACATATAAAGCGAAGCTTCCCTTACAAATTAT
CCTGATGATACTGAACTCCATTGCCTTCCAAAATTCCGCCATTGATTGGGTGAGGGAC
CACCGTCTCCATCATAAGTACAGTGACACTGATGCAGACCCTCACAATGCTACTCGTG
GTTTCTTCTATTCTCATGTTGGATGGTTGCTCGTAAGAAAACATCCAGAAGTCAAGAGA
CGTGGAAAGGAACTTGACATGTCTGATATTTACAACAATCCAGTGCTGAGATTTCAAAA
GAAGTATGCTATACCCTTCATCGGGGCAATGTGCTTCGGATTACCAACTTTTATCCCTG
TTTACTTCTGGGGAGAAACCTGGAGTAATGCTTGGCATATCACCATGCTTCGGTACAT
CCTCAACCTAAACATTACTTTCCTGGTCAACAGTGCTGCTCATATCTGGGGATACAAAC
CTTATGACATCAAAATATTGCCTGCCCAAAATATAGCAGTTTCCATAGTAACCGGCGGC
GAAGTTTCCATAACTACCACCACGTTTTTTCCTTGGGATTATCGTGCAGCAGAATTGGG
GAACAATTATCTTAATTTGACGACTAAGTTCATAGATTTCTTCGCTTGGATCGGATGGG
CTTACGATCTTAAGACGGTGTCCAGTGATGTTATAAAAAGTAAGGCGGAAAGAACTGG
TGATGGGACGAATCTTTGGGGTTTAGAAGACAAAGGTGAAGAAGATTTTTTGAAAATCT
GGAAAGACAATTAA
SEQ ID NO: 43: As_Δ11-desaturase amino acid sequence
MAQGVQTTTILREEEPSLTFVVPQEPRKYQIVYPNLITFGYWHIAGLYGLYLCFTSAKWQTI
LFSFMLVVLAELGITAGAHRLWAHKTYKAKLPLQIILMILNSIAFQNSAIDWVRDHRL
HHKYSDTDADPHNATRGFFYSHVGWLLVRKHPEVKRRGKELDMSDIYNNPVLRFQKKYAI
PFIGAMCFGLPTFIPVYFWGETWSNAWHITMLRYILNLNITFLVNSAAHIWGYKPYDIKILPA
QNIAVSIVTGGEVSITTTTFFPWDYRAAELGNNYLNLTTKFIDFFAWIGWAYDLKTVSSDVIK
SKAERTGDGTNLWGLEDKGEEDFLKIWKDN
SEQ ID NO: 44: Tni_Δ11-desaturase DNA sequence; mRNA-coding sequence.
ATGGCTGTGATGGCTCAAACAGTACAAGAAACGGCTACAGTGTTGGAAGAGGAAGCT
CGCACAGTGACTCTTGTGGCTCCAAAGACAACGCCAAGGAAATATAAATATATATACAC
CAACTTTCTTACATTTTCATATGCGCATTTAGCTGCATTATACGGACTTTATTTGTGCTT
CACCTCTGCGAAATGGGAAACATTGCTATTCTCTTTCGTACTCTTCCACATGTCAAATA
TAGGCATCACCGCAGGGGCTCACCGACTCTGGACTCACAAGACTTTCAAAGCCAAATT
GCCTTTGGAAATTGTCCTCATGATATTCAACTCTTTAGCCTTTCAAAACACGGCTATTAC
ATGGGCTAGAGAACATCGGCTACATCACAAATACAGCGATACTGATGCTGATCCCCAC
AATGCGTCAAGAGGGTTCTTCTACTCGCATGTTGGCTGGCTATTAGTAAAAAAACATCC
CGATGTCCTGAAATATGGAAAAACTATAGACATGTCGGATGTATACAATAATCCTGTGT
TAAAATTTCAGAAAAAGTACGCAGTACCCTTAATTGGAACAGTTTGTTTTGCTCTTCCAA
CTTTGATTCCAGTCTACTGTTGGGGCGAATCGTGGAACAACGCTTGGCACATAGCCTT
ATTTCGATACATATTCAATCTTAACGTGACTTTCCTAGTCAACAGTGCTGCGCATATCT
GGGGGAATAAGCCTTATGATAAAAGCATCTTGCCCGCTCAAAACCTGCTGGTTTCCTT
CCTAGCAAGTGGAGAAGGCTTCCATAATTACCATCACGTCTTTCCATGGGATTACCGC
ACAGCAGAATTAGGGAATAACTTCCTGAATTTGACGACGCTGTTCATTGATTTTTGTGC
CTGGTTTGGATGGGCTTATGACTTGAAGTCTGTATCAGAGGATATTATAAAACAGAGAG
CTAAACGAACAGGTGACGGTTCTTCAGGGGTCATTTGGGGATGGGACGACAAAGACA
TGGACCGCGATATAAAATCTAAAGCTAACATTTTTTATGCTAAAAAGGAATGA
SEQ ID NO: 45: Tni_Δ11-desaturase amino acid sequence
MAVMAQTVQETATVLEEEARTVTLVAPKTTPRKYKYIYTNFLTFSYAHLAALYGLYLCFTSA
KWETLLFSFVLFHMSNIGITAGAHRLWTHKTFKAKLPLEIVLMIFNSLAFQNTAITWAREHRL
HHKYSDTDADPHNASRGFFYSHVGWLLVKKHPDVLKYGKTIDMSDVYNNPVLKFQKKYA
VPLIGTVCFALPTLIPVYCWGESWNNAWHIALFRYIFNLNVTFLVNSAAHIWGNKPYDKSIL
PAQNLLVSFLASGEGFHNYHHVFPWDYRTAELGNNFLNLTTLFIDFCAWFGWAYDLKSVS
EDIIKQRAKRTGDGSSGVIWGWDDKDMDRDIKSKANIFYAKKE
SEQ ID NO: 45: Sc_FAA DNA sequence; mRNA coding sequence
atggccgctccagattatgcacttaccgatttaattgaatcggatcctcgtttcgaaagtttgaagacaagattagccggttacacc
aaaggctctgatgaatatattgaagagctatactctcaattaccactgaccagctaccccaggtacaaaacatttttaaagaaac
aggcggttgccatttcgaatccggataatgaagctggttttagctcgatttataggagttctctttcttctgaaaatctagtgagctgtg
tggataaaaacttaagaactgcatacgatcacttcatgttttctgcaaggagatggcctcaacgtgactgtttaggttcaaggcca
attgataaagccacaggcacctgggaggaaacattccgtttcgagtcgtactccacggtatctaaaagatgtcataatatcgga
agtggtatattgtctttggtaaacacgaaaaggaaacgtcctttggaagccaatgattttgttgttgctatcttatcacacaacaacc
ctgaatggatcctaacagatttggcctgtcaggcctattctctaactaacacggctttgtacgaaacattaggtccaaacacctcc
gagtacatattgaatttaaccgaggcccccattctgatttttgcaaaatcaaatatgtatcatgtattgaagatggtgcctgatatga
aatttgttaatactttggtttgtatggatgaattaactcatgacgagctccgtatgctaaatgaatcgttgctacccgttaagtgcaact
ctctcaatgaaaaaatcacatttttttcattggagcaggtagaacaagttggttgctttaacaaaattcctgcaattccacctacccc
agattccttgtatactatttcgtttacttctggtactacaggtttacctaaaggtgtggaaatgtctcacagaaacattgcgtctgggat
agcatttgctttttctaccttcagaataccgccagataaaagaaaccaacagttatatgatatgtgttttttgccattggctcatatttttg
aaagaatggttattgcgtatgatctagccatcgggtttggaataggcttcttacataaaccagacccaactgtattggtagaggatt
tgaagattttgaaaccttacgcggttgccctggttcctagaatattaacacggtttgaagccggtataaaaaatgctttggataaat
cgactgtccagaggaacgtagcaaatactatattggattctaaatcggccagatttaccgcaagaggtggtccagataaatcga
ttatgaattttctagtttatcatcgcgtattgattgataaaatcagagactctttaggtttgtccaataactcgtttataattaccggatca
gctcccatatctaaagataccttactatttttaagaagcgccttggatattggtataagacagggctacggcttaactgaaacttttg
ctggtgtctgtttaagcgaaccgtttgaaaaagatgtcggatcttgtggtgccataggtatttctgcagaatgtagattgaagtctgtt
ccagaaatgggttaccatgccgacaaggatttaaaaggtgaactgcaaattcgtggcccacaggtttttgaaagatattttaaaa
atccgaatgaaacttcaaaagccgttgaccaagatggttggttttccacgggagatgttgcatttatcgatgcaaaaggtcgcatc
agcgtcattgatcgagtcaagaactttttcaagctagcacatggtgaatatattgctccagagaaaatcgaaaatatttatttatcat
catgcccctatatcacgcaaatatttgtctttggagatcctttgaagacatttttagttggcatcgttggtgttgatgttgatgcagcgca
accgattttagctgcaaagcacccagaggtgaaaacgtggactaaggaagtgctagtagaaaacttaaatcgtaataaaaag
ctaaggaaggaatttttaaacaaaattaataaatgcatcgatgggctacaaggatttgaaaaattgcacaacatcaaagtcgga
cttgagcctttgactctcgaggatgatgttgtgacgccaacttttaaaataaagcgtgccaaagcatcaaaattcttcaaagatac
attagaccaactatacgccgaaggttcactagtcaagacagaaaagctttag
SEQ ID NO: 47: Sc_FAA2 amino acid sequence
MAAPDYALTDLIESDPRFESLKTRLAGYTKGSDEYIEELYSQLPLTSYPRYKTFLKKQAVAIS
NPDNEAGFSSIYRSSLSSENLVSCVDKNLRTAYDHFMFSARRWPQRDCLGSRPIDKATGT
WEETFRFESYSTVSKRCHNIGSGILSLVNTKRKRPLEANDFVVAILSHNNPEWILTDLACQA
YSLTNTALYETLGPNTSEYILNLTEAPILIFAKSNMYHVLKMVPDMKFVNTLVCMDELTHDE
LRMLNESLLPVKCNSLNEKITFFSLEQVEQVGCFNKIPAIPPTPDSLYTISFTSGTTGLPKGV
EMSHRNIASGIAFAFSTFRIPPDKRNQQLYDMCFLPLAHIFERMVIAYDLAIGFGIGFLHKPD
PTVLVEDLKILKPYAVALVPRILTRFEAGIKNALDKSTVQRNVANTILDSKSARFTARGGPDK
SIMNFLVYHRVLIDKIRDSLGLSNNSFIITGSAPISKDTLLFLRSALDIGIRQGYGLTETFAGVC
LSEPFEKDVGSCGAIGISAECRLKSVPEMGYHADKDLKGELQIRGPQVFERYFKNPNETSK
AVDQDGWFSTGDVAFIDAKGRISVIDRVKNFFKLAHGEYIAPEKIENIYLSSCPYITQIFVFG
DPLKTFLVGIVGVDVDAAQPILAAKHPEVKTWTKEVLVENLNRNKKLRKEFLNKINKCIDGL
QGFEKLHNIKVGLEPLTLEDDVVTPTFKIKRAKASKFFKDTLDQLYAEGSLVKTEKL

### REFERENCES

Alfaro, Navarro-Llopis, Primo, 2009. Optimization of pheromone dispenser density for managing the rice striped stem borer, Chilo suppressalis (Walker), by mating disruption. Crop Protection. 28:567-572.
Angerer, Radermacher, Mankowska, Steger, Zwicker, Heide, Wittig, Brandt, Zickermann, 2014. The LYR protein subunit NB4M/NDUFA6 of mitochondrial complex I anchors an acyl carrier protein and is essential for catalytic activity. PNAS. 111(14)
Bari, 2003. Development of pheromone mating disruption strategies for the suppression of the artichoke plume moth in artichokes grown on the central coast of California. ISHS Acta Horticulturae 660: V International Congress on Artichoke. doi: 10.17660/ActaHortic.2004.660.80
Chen, Beckerich, Gaillardin, 1997. One-step transformation of the dimorphic yeast Yarrowia lipolytica. Appl Microbiol Biotechnol. 48(2):232-5
Eizaguirre, Sans, Lopez, Albajes. 2002. Effects of mating disruption against the Mediterranean corn borer, Sesamia nonagrioides, on the European corn borer Ostrinia nubilalis. Use of pheromones and other semiochemicals in integrated production IOBC wprs Bulletin.
Ferrell, Yao, 1972. Reductive and oxidative synthesis of saturated and unsaturated fatty aldehydes, J Lipid Res. 13(1):23-6.).
Gietz R D & Schiestl R H, 2007. Quick and easy yeast transformation using the LiAc/SS carrier DNA/PEG method. Nat Protoc 2: 35-37.
Hagstrom, Wang, Lienard, Lassance, Johansson, Lofstedt, 2013. A moth pheromone brewery: production of (Z)-11-hexadecenol by heterologous co-expression of two biosynthetic genes from a noctuid moth in a yeast cell factory. Microbial Cell Factories 12:125
Jensen, Strucko, Kildegaard, David, Maury, Mortensen, Forster, Nielsen, Borodina, 2014. EasyClone: method for iterative chromosomal integration of multiple genes in Saccharomyces cerevisiae, FEMS Yeast Res. 14(2):238-48
Kehat, Dunkelblum, 1993. Sex Pheromones: achievements in monitoring and mating disruption of cotton pests in Israel, Achieves of Insect Biochemistry and Physiology. 22:425-431.
Li, Zhang, 2009. An environmentally benign TEMPO-catalyzed efficient alcohol oxidation system with a recyclable hypervalent iodine(III) reagent andilts facile preparation. Synthesis, 1163-1169a.
Maury, Germann, Baallal Jacobsen, Jensen, Kildegaard, HerrOrd, Schneider, Koza, Forster, Nielsen, Borodina, 2016. EasyCloneMulti: A Set of Vectors for Simultaneous and Multiple Genomic Integrations in Saccharomyces cerevisiae. PLoS One. 11(3):e0150394
Meyer, Schreiber, 1994. Acceleration of the Dess-Martin oxidation by water J. Org. Chem., 59, 7549-7552;
Okada, Asawa, Sugiyama, Kirihara, Iwai, Kimura, 2014. Sodium hypochlorite pentahydrate (NaOCl·5H2O) crystals as an extraordinary oxidant for primary and secondary alcohols. Synlett, 25, 596-598.
Stovicek, Borja, Forster, Borodina, 2015. EasyClone 2.0: expanded toolkit of integrative vectors for stable gene expression in industrial Saccharomyces cerevisiae strains. J Ind Microbiol Biotechnol, 42, 1519-31
Tamura, Aoyama, Takido, Kodomari, 2012. Novel [4-Hydroxy-TEMPO+NaCl]/SiO2 as a reusable catalyst for aerobic oxidation of alcohols to carbonyls. Synlett, 23, 1397-1407.
Yadav, Reddy, Basak, Narsaiah, 2004. Recyclable 2nd generation ionic liquids as green solvents for the oxidation of alcohols with hypervalent iodine reagents, Tetrahedron, 60, 2131-2135.
Wu, Zhang, Yao, Xu, Wang and Zhang, 2012. Management of diamondback moth, Plutella xylostella (Lepidoptera: Plutellidae) by mating disruption. Insect Science 19 (6), 643-648.

1. A method for production of a moth pheromone in a yeast cell, said method comprising the steps of:
i) providing a yeast cell capable of synthesising hexadecanoyl-CoA, said yeast cell expressing:
a Δ11-desaturase selected from the group consisting of a Δ11-desaturase comprising SEQ ID NO: 2, a Δ11-desaturase comprising SEQ ID NO: 41, a Δ11-desaturase comprising SEQ ID NO: 43, and a Δ11-desaturase comprising SEQ ID NO: 45 or a Δ11-desaturase variant having at least 85% sequence identity to the polypeptide of SEQ ID NO: 2, SEQ ID NO: 41, SEQ ID NO: 43, or SEQ ID NO: 45, said Δ11-desaturase variant having Δ11-desaturase activity, and
an alcohol-forming fatty acyl-CoA reductase (FAR) selected from the group consisting of a FAR comprising SEQ ID NO: 8, a FAR comprising SEQ ID NO: 16, and a FAR comprising SEQ ID NO: 12 or a FAR variant having at least 85% sequence identity to the polypeptide of SEQ ID NO: 8, or SEQ ID NO: 12 or a FAR variant having at least 99% sequence identity to the polypeptide of SEQ ID NO: 16, said FAR variant having alcohol-forming fatty acyl-CoA reductase activity;
ii) expressing said Δ11-desaturase and said FAR from said yeast cell; and
iii) incubating said yeast cell in a medium,
whereby
the Δ11-desaturase is capable of converting at least part of said hexadecanoyl-CoA to (Z)11-hexadecenoyl-CoA; and
said FAR is capable of converting at least part of said (Z)11-hexadecenoyl-CoA to (Z)-11-hexadecen-1-ol,
thereby obtaining a moth pheromone comprising (Z)-11-hexadecen-1-ol with a titre of at least 1 mg/L, wherein the titre is measured in a culture medium after culturing the yeast cell.

2. The method of claim 1, wherein said yeast cell is a genus selected from the group consisting of Saccharomyces, Pichia, Yarrowia, Kluyveromyces, Candida, Rhodotorula, Rhodosporidium, Cryptococcus, Trichosporon and Lipomyces.

3. The method of claim 1, wherein the yeast cell is selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.

4. The method of claim 1, wherein the yeast cell is further capable of expressing a fatty acyl-CoA synthetase (FAA).

5. The method of claim 4, wherein the FAA is selected from the group consisting of a FAA comprising SEQ ID NO: 35, a FAA comprising SEQ ID NO: 37, and a FAA variant having at least 85% sequence identity to the polypeptide of SEQ ID NO: 35 or SEQ ID NO: 37, said FAA variant having fatty acyl-CoA synthetase activity.

6. The method of claim 1, further comprising the step of converting at least part of the produced (Z)-11-hexadecen-1-ol into (Z)-11-hexadecen-1-yl acetate by expression of an acetyltransferase or by chemical conversion.

7. The method of claim 6, wherein the acetyltransferase is a heterologous acetyltransferase (AcT) expressed from said yeast cell or a native acetyltransferase overexpressed from said yeast cell compared to the native expression levels of said native acetyltransferase in said yeast cell, wherein said acetyltransferase is capable of converting at least part of the (Z)-11-hexadecen-1-ol into (Z)11-hexadecen-1-yl acetate, thereby further producing (Z)11-hexadecen-1-yl acetate.

8. The method of claim 6, wherein the acetyltransferase is selected from the group consisting of an acetyltransferase comprising SEQ ID NO: 39 and (ii) an acetyltransferase variant having at least 85% sequence identity to the polypeptide of SEQ ID NO: 39, said acetyltransferase variant having acetyltransferase activity.

9. The method of claim 1, wherein:
the Δ11-desaturase is encoded by a nucleic acid sequence identical to or having at least 85% identity to SEQ ID NO: 1, SEQ ID NO: 40, SEQ ID NO: 42 or SEQ ID NO: 44; and/or
the FAR is encoded by a nucleic acid sequence identical to or having at least 90% identity to SEQ ID NO: 7, at least 90% identity to SEQ ID NO: 11 or at least 90% identity to SEQ ID NO: 15 or 100% identity to SEQ ID NO: 7, SEQ ID NO: 11 or SEQ ID NO: 15.

10. The method of claim 1, further comprising the step of converting at least part of the produced (Z)-11-hexadecen-1-ol into (Z)-11-hexadecenal by a step of chemical conversion.

11. The method of claim 1, said method further comprising the step of recovering (Z)-11-hexadecen-1-ol to produce recovered (Z)-11-hexadecen-1-ol.

12. The method of claim 11, said method further comprising the step of formulating the recovered (Z)-11-hexadecen-1-ol into a pheromone composition.

13. The method of claim 1, further comprising the step of converting at least part of the (Z)-11-hexadecen-1-ol into (Z)11-hexadecen-1-yl acetate by a step of chemical conversion.

14. The method of claim 13, said method further comprising the step of recovering (Z)11-hexadecen-1-yl acetate to product recovered (Z)11-hexadecen-1-yl acetate.

15. The method of claim 14, said method further comprising the step of formulating the recovered (Z)11-hexadecen-1-yl acetate into a pheromone composition.

16. The method of claim 1, wherein the Δ11-desaturase is a Δ11-desaturase variant having at least 85% sequence identity to the polypeptide of SEQ ID NO: 41, said Δ11-desaturase variant having Δ11-desaturase activity.

17. The method of claim 16, wherein the fatty acyl-CoA reductase (FAR) is a FAR variant having at least 85% sequence identity to the polypeptide of SEQ ID NO: 8, said FAR variant having alcohol-forming fatty acyl-CoA reductase activity.

18. The method of claim 1, wherein the Δ11-desaturase is a Δ11-desaturase variant having at least 85% sequence identity to the polypeptide of SEQ ID NO: 2, said Δ11-desaturase variant having Δ11-desaturase activity.

19. The method of claim 1, wherein the fatty acyl-CoA reductase (FAR) is a FAR variant having at least 85% sequence identity to the polypeptide of SEQ ID NO: 8, said FAR variant having alcohol-forming fatty acyl-CoA reductase activity.

20. A yeast cell capable of synthesising hexadecanoyl-CoA, said yeast cell further capable of expressing:
a Δ11-desaturase selected from the group consisting of a Δ11-desaturase comprising SEQ ID NO: 2, a Δ11-desaturase comprising SEQ ID NO: 41, a Δ11-desaturase comprising SEQ ID NO: 43, and a Δ11-desaturase comprising SEQ ID NO: 45 or a Δ11-desaturase variant having at least 85% sequence identity to the polypeptide of SEQ ID NO: 2, SEQ ID NO: 41, SEQ ID NO: 43, or SEQ ID NO: 45, said Δ11-desaturase variant having Δ11-desaturase activity, and
an alcohol-forming fatty acyl-CoA reductase (FAR) selected from the group consisting of a FAR comprising SEQ ID NO: 8, a FAR comprising SEQ ID NO: 16, and a FAR comprising SEQ ID NO: 12 or a FAR variant having at least 85% sequence identity to the polypeptide of SEQ ID NO: 8, or SEQ ID NO: 12 or a FAR variant having at least 99% sequence identity to the polypeptide of SEQ ID NO: 16, said FAR variant having alcohol-forming fatty acyl-CoA reductase activity.
